(19) United States
(12) Patent Application Publication
(10) Pub. No.: US 2026/0028310 A1
(43) Pub. Date: Jan. 29, 2026

(54) LIPID COMPOUND AND USES THEREOF

(71) Applicant: LONGUIDE BIOPHARMA CORPORATION, Shenzhen (CN)

(72) Inventors: Longgui ZHANG, Shenzhen (CN); Yuebao ZHANG, Shenzhen (CN); Chen LIU, Shenzhen (CN); Meigui LIANG, Shenzhen (CN)

(21) Appl. No.: 19/349,653

(22) Filed: Oct. 3, 2025

Related U.S. Application Data
(63) Continuation-in-part of application No. PCT/CN2024/124839, filed on Oct. 15, 2024.

(30) Foreign Application Priority Data
Nov. 8, 2023 (CN) 202311483709.7

(51) Int. Cl. C07C 219/06; A61K 9/51; A61K 31/7105; A61K 31/711; A61K 31/713; C07D 295/13

(52) U.S. Cl. CPC C07C 219/06; A61K 9/5123; A61K 31/7105; A61K 31/711; A61K 31/713; C07D 295/13

(57) ABSTRACT

The invention relates to a compound, a lipid compound nanoparticle, a nucleic acid nanoparticle complex, a pharmaceutical composition and uses thereof in the drug delivery field, and belongs to the fields of biomedicine and biotechnology. The structure of the compound is shown as formula A. The compound, lipid compound nanoparticle or nucleic acid nanoparticle complex provided herein has the advantages of good biocompatibility, high transfection efficiency, low toxicity and excellent technical effects.

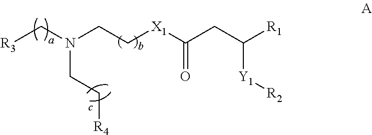

This application is a continuation-in-part of International Application No. PCT/CN2024/124839, filed Oct. 15, 2024, which claims priority to and the benefit of Chinese patent application No. 202311483709.7, filed on Nov. 8, 2023. The contents of each of which are hereby incorporated by reference in their entirety.

### TECHNICAL FIELD

The invention belongs to the field of biological medicine and biotechnology, specifically relates to lipid compound and uses thereof.

### BACKGROUND ART

Gene transfection is a technique of transferring or transporting nucleic acid with biologic function into cells and maintaining its biological function in the cells. A gene vector refers to a tool for introducing an exogenous therapeutic gene into a biological cell. Currently, gene vectors having industrial transformation potential internationally mainly include viral vectors and non-viral vectors.

Viral vectors are gene delivery vehicles based on the virus's ability to transmit its genome into other cells for infection. The current viral vectors with better application prospects comprise lentiviral vectors, adenoviral vectors, retroviral vectors, adeno-associated viral vectors and so on. However, due to its inherent physicochemical properties and biological activity, viral vectors have serious disadvantages such as high production cost, limited loading capacity, poor targeting property, insertion integration, teratogenic mutagenesis, etc., which are disadvantageous for the development of universal and general therapies.

Non-viral vectors mainly include: liposome nanoparticles, complex nanoparticles, cationic polymer nanoparticles, polypeptide nanoparticles and the like. The liposome nanoparticle is a main non-viral vector applied to RNA drug development at present, and the first RNAi drug (Patisiran) and the first mRNA drug (BNT 162b2, Comirnaty) are granted sequentially at present, the clinical application value of the Liposome Nanoparticle (LNP) is fully validated. Compared with viral vectors, liposome nanoparticles have the advantages of low production cost, definite chemical structure, convenience in quality control, realization of targeted drug delivery through targeted modification, theoretically unlimited loading capacity and the like, but most liposome lipid materials are not degradable and have high toxicity, therefore, it is difficult to meet the clinical needs of repeated administration, and in addition, the problems of poor in vivo transfection effect, metabolism or elimination of nucleic acid in serum, low bioavailability and the like exist.

Therefore, there is still a need for nanoparticles with good biocompatibility and high transfection efficiency.

### SUMMARY OF THE INVENTION

In order to solve the above technical problems, the present invention provides the following technical solutions.

In a first aspect, the present invention provides a compound of formula A,

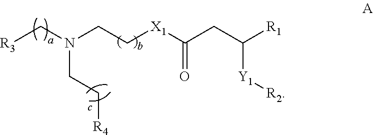

In a second aspect, the present invention provides a lipid compound nanoparticle.

The lipid compound nanoparticle comprises the compound of the first aspect and auxiliary materials, or comprises the compound of the first aspect, nucleic acid and auxiliary materials.

In a third aspect, the present invention provides a nucleic acid nanoparticle complex. The nucleic acid nanoparticle complex comprises a nucleic acid and at least one lipid compound nanoparticle of the second aspect.

In a fourth aspect, the present invention provides a pharmaceutical composition. The pharmaceutical composition comprises the lipid compound nanoparticle of the second aspect or the nucleic acid nanoparticle complex of the third aspect, and a pharmaceutically acceptable adjuvant.

In a fifth aspect, the present invention provides use of the compound of the first aspect or the lipid compound nanoparticle of the second aspect or the nucleic acid nanoparticle complex of the third aspect or the pharmaceutical composition of the fourth aspect in the manufacture of a product for delivering nucleic acids in vivo.

The compound, lipid compound nanoparticle or nucleic acid nanoparticle complex provided herein has the advantages of good biocompatibility, high transfection efficiency, low toxicity and excellent technical effects.

### DETAILED DESCRIPTION

In order to solve the above technical problems, the present invention provides the following technical solutions.

In a first aspect, the present invention provides a compound.

A compound of formula A,

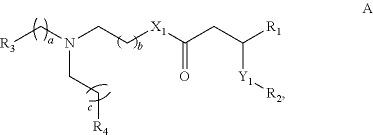

wherein a is an integer from 0 to 10; (i.e. 0, 1, 2, 3, 4, 5, 6, 7, 8, 9 or 10);
b is an integer from 1 to 9; (i.e. 1, 2, 3, 4, 5, 6, 7, 8 or 9);
c is an integer from 1 to 9; (i.e. 1, 2, 3, 4, 5, 6, 7, 8 or 9);
R1 is C1˜C20 alkyl (i.e. C1 alkyl, C2 alkyl, C3 alkyl, C4 alkyl, C5 alkyl, C6 alkyl, C7 alkyl, C8 alkyl, C9 alkyl, C10 alkyl, C11 alkyl, C12 alkyl, C13 alkyl, C14 alkyl, C15 alkyl, C16 alkyl, C17 alkyl, C18 alkyl, C19 alkyl or C20alkyl);
R2 is hydrogen or —C(═O)Ra;
Ra is C1˜C20 alkyl (i.e. C1 alkyl, C2 alkyl, C3 alkyl, C4 alkyl, C5 alkyl, C6 alkyl, C7alkyl, C8 alkyl, C9 alkyl, C10 alkyl, C11 alkyl, C12 alkyl, C13 alkyl, C14 alkyl, C15 alkyl, C16 alkyl, C17 alkyl, C18 alkyl, C19 alkyl or C20alkyl);
R3 is hydroxy,

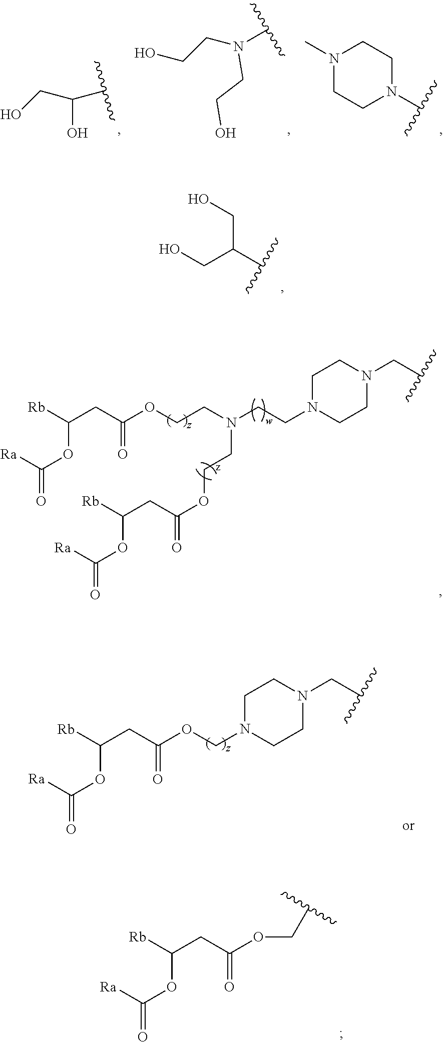

R4 is

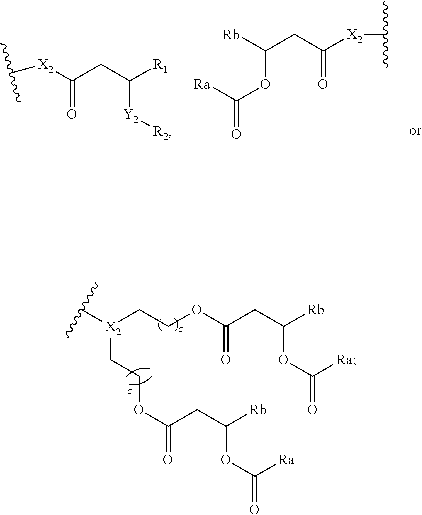

Rb is C1-C20 alkyl (i.e. C1 alkyl, C2 alkyl, C3 alkyl, C4 alkyl, C5 alkyl, C6 alkyl, C7 alkyl, C8 alkyl, C9 alkyl, C10 alkyl, C11 alkyl, C12 alkyl, C13 alkyl, C14 alkyl, C15 alkyl, C16 alkyl, C17 alkyl, C18 alkyl, C19 alkyl or C20alkyl);
w is an integer from 1 to 9; (i.e. 1, 2, 3, 4, 5, 6, 7, 8 or 9);
z is an integer from 1 to 9; (Le. 1, 2, 3, 4, 5, 6, 7, 8 or 9);
X1 is O or NH;
each X2 is independently O or NH;
Y1 is O or NH;
Y2 is O or NH.

In some embodiments, the compound of formula A is selected from a compound of formula I, a compound of formula II, a compound of formula III and a compound of formula IV,

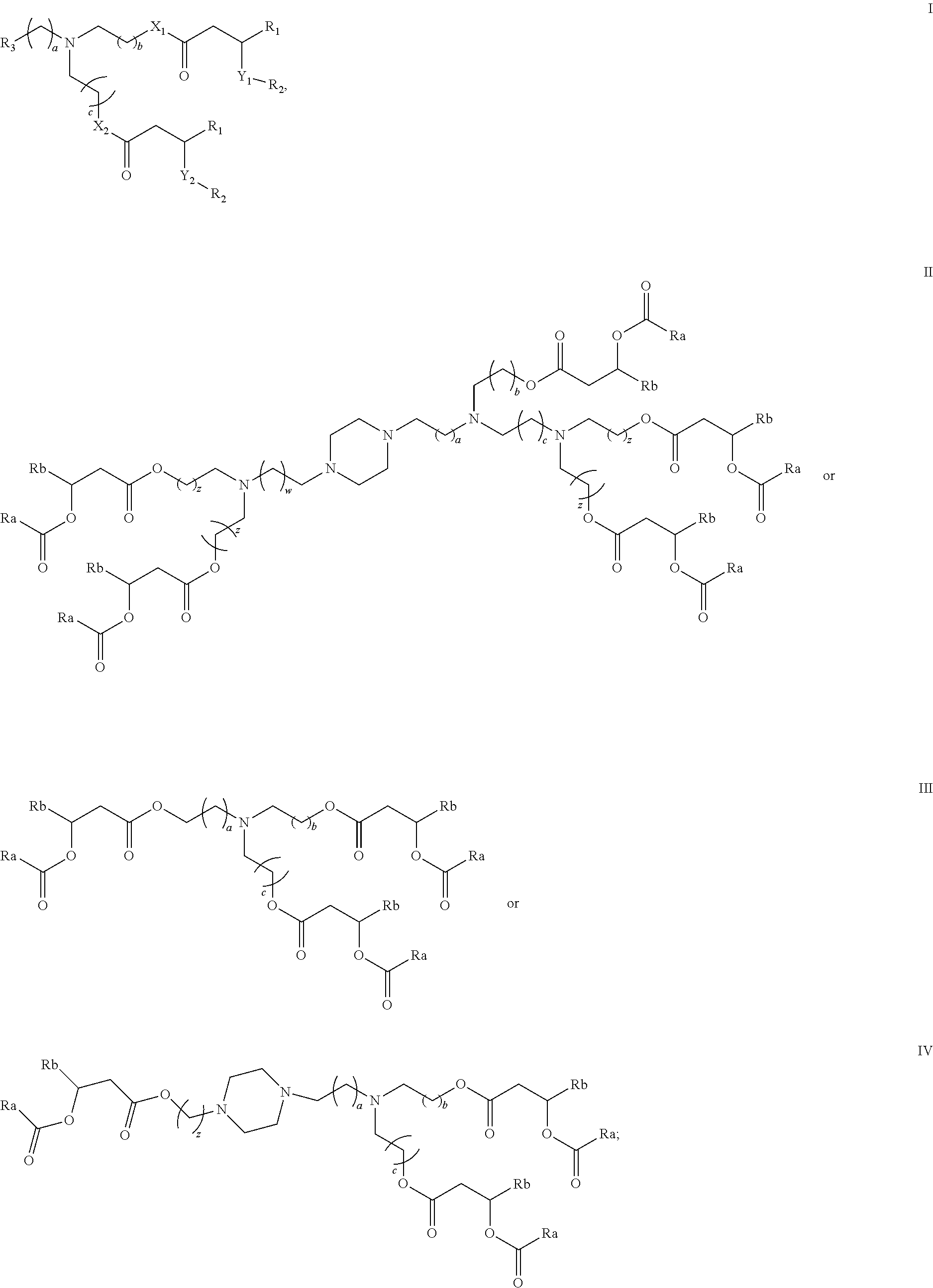

wherein in the compound of formula II, w is an integer from 1 to 9 (i.e. 1, 2, 3, 4, 5, 6, 7, 8 or 9); z is an integer from 1 to 9 (i.e. 1, 2, 3, 4, 5, 6, 7, 8 or 9);
in the compound of formula I, R3 is hydroxy,

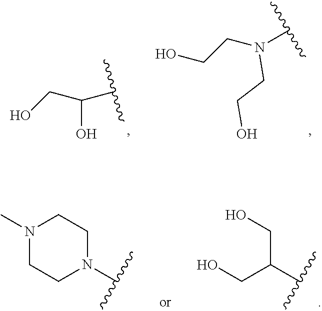

In some embodiments, in the compound of formula A or in the compound of formula I, X1 is O; each X2 is independently O or NH; Y1 is O or NH; Y2 is O or NH.

In some embodiments, in the compound of formula A or in the compound of formula I, X1 is O; each X2 is O; Y1 is O; Y2 is O.

In some embodiments, in the compound of formula A or in the compound of formula I, X1 is NH; each X2 is independently O or NH; Y1 is O or NH; Y2 is O or NH.

In some embodiments, in the compound of formula A or in the compound of formula I, X1 is NH; each X2 is NH; Y1 is NH; Y2 is NH. In some embodiments, the compound of formula A is selected from a compound of formula I, wherein R1 is unsubstituted C1-C15 straight chain alkyl (i.e. unsubstituted C1 straight chain alkyl, unsubstituted C2 straight chain alkyl, unsubstituted C3 straight chain alkyl, unsubstituted C4 straight chain alkyl, unsubstituted C5 straight chain alkyl, unsubstituted C6 straight chain alkyl, unsubstituted C7 straight chain alkyl, unsubstituted C8 straight chain alkyl, unsubstituted C9 straight chain alkyl, unsubstituted C10 straight chain alkyl, unsubstituted C11 straight chain alkyl, unsubstituted C12 straight chain alkyl, unsubstituted C13 straight chain alkyl, unsubstituted C14 straight chain alkyl, unsubstituted C15 straight chain alkyl); R2 is hydrogen or —C(═O)Ra; Ra is unsubstituted C5-C15 straight chain alkyl (i.e. unsubstituted C5 straight chain alkyl, unsubstituted C6 straight chain alkyl, unsubstituted C7 straight chain alkyl, unsubstituted C8 straight chain alkyl, unsubstituted C9 straight chain alkyl, unsubstituted C10 straight chain alkyl, unsubstituted C11 straight chain alkyl, unsubstituted C12 straight chain alkyl, unsubstituted C13 straight chain alkyl, unsubstituted C14 straight chain alkyl, unsubstituted C15 straight chain alkyl); a is an integer from 0 to 6 (i.e. 0, 1, 2, 3, 4, 5 or 6); b is an integer from 3 to 5 (i.e. 3, 4 or 5); c is an integer from 3 to 5 (i.e. 3, 4 or 5).

In some embodiments, the compound of formula A is selected from a compound of formula I, wherein R1 is unsubstituted C5-C11 straight chain alkyl (i.e. unsubstituted C5 straight chain alkyl, unsubstituted C6 straight chain alkyl, unsubstituted C7 straight chain alkyl, unsubstituted C8 straight chain alkyl, unsubstituted C9 straight chain alkyl, unsubstituted C10 straight chain alkyl, unsubstituted C11 straight chain alkyl); R2 is hydrogen or —C(═O)Ra; Ra is unsubstituted C5-C13 straight chain alkyl (i.e. unsubstituted C5 straight chain alkyl, unsubstituted C6 straight chain alkyl, unsubstituted C7 straight chain alkyl, unsubstituted C8 straight chain alkyl, unsubstituted C9 straight chain alkyl, unsubstituted C10 straight chain alkyl, unsubstituted C11 straight chain alkyl, unsubstituted C12 straight chain alkyl, unsubstituted C13 straight chain alkyl); a is an integer from 0 to 6 (i.e. 0, 1, 2, 3, 4, 5 or 6); b is an integer from 3 to 5 (i.e. 3, 4 or 5); c is an integer from 3 to 5 (i.e. 3, 4 or 5).

In some embodiments, the compound of formula A is selected from a compound of formula I, the compound of formula I is selected from compound L0111, compound L0112, compound L0113, compound L0114, compound L0115, compound L0116, compound L0117, compound L0118, compound L0119, compound L0120, compound L0121, compound L0122, compound L0123, compound L0124, compound L0125, compound L0132, compound L0133, compound L0134, compound L0135, compound L0136, compound L0137, compound L0138, compound L0139 and compound L0140,

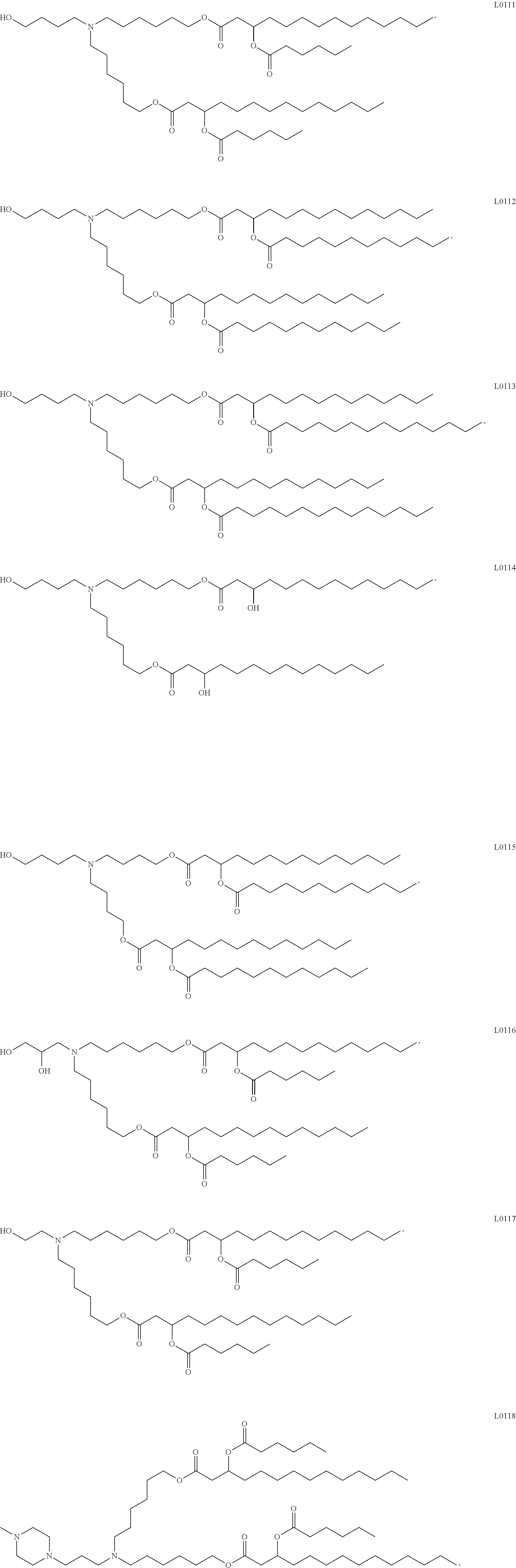

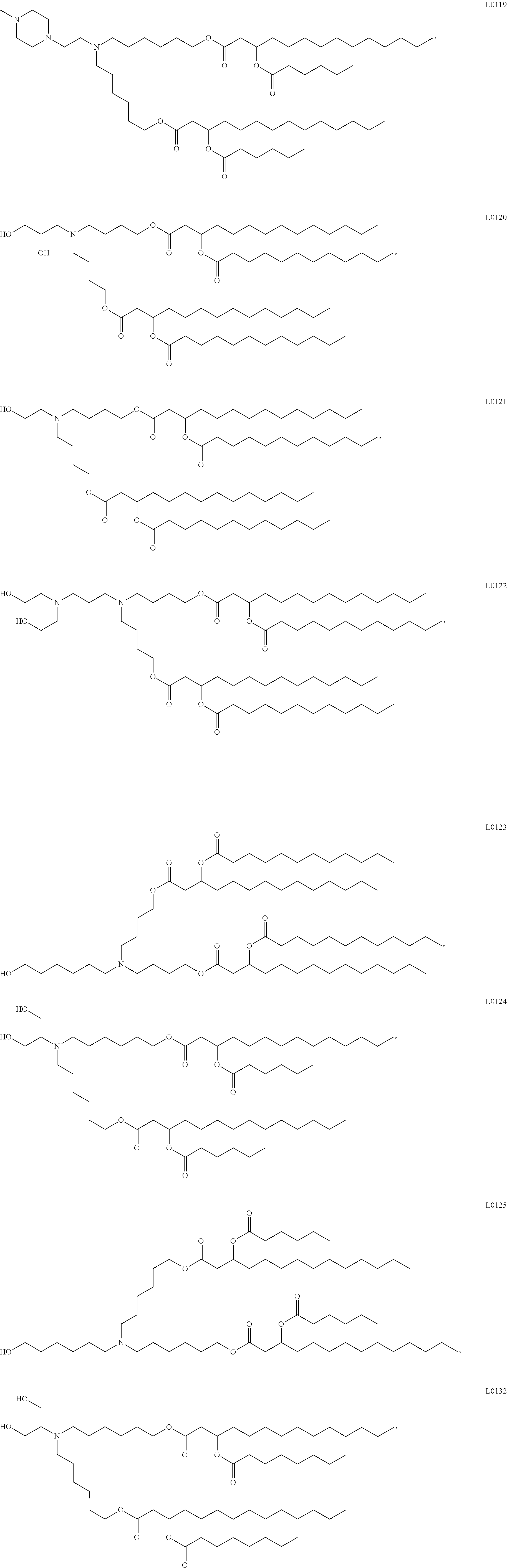

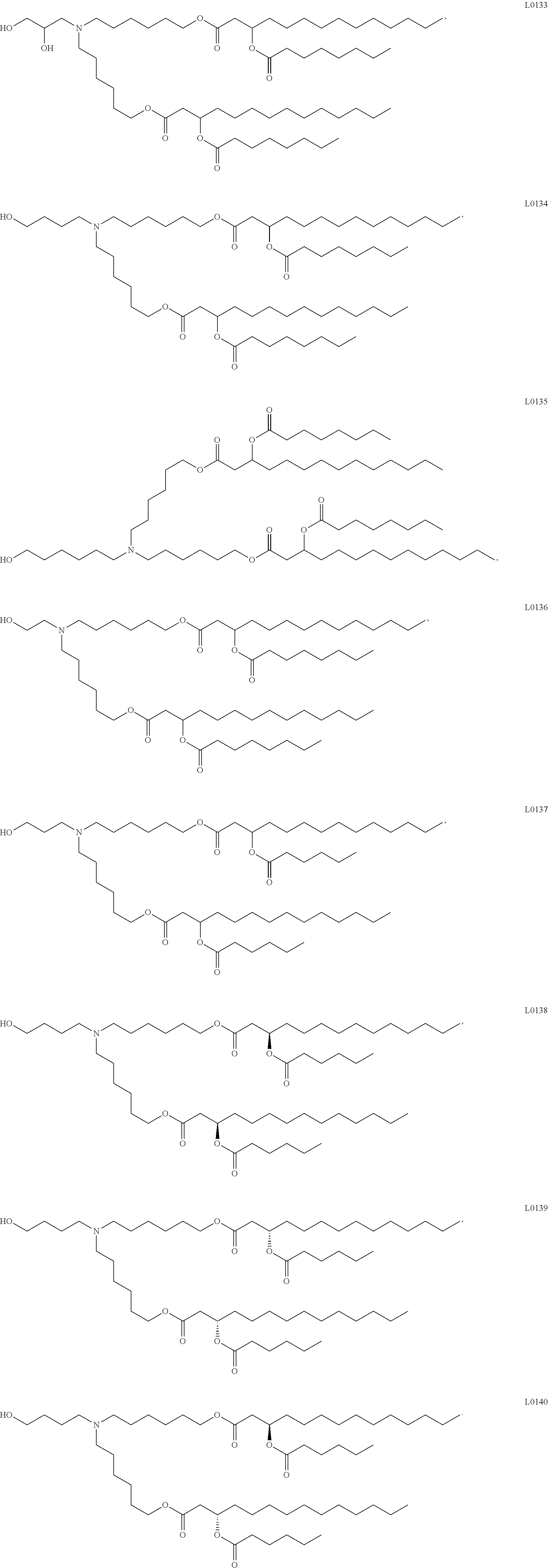

In some embodiments, the compound of formula A is selected from a compound of formula II, wherein a is an integer from 0 to 5 (i.e. 0, 1, 2, 3, 4 or 5); b is an integer from 3 to 10 (i.e. 3, 4, 5, 6, 7, 8, 9 or 10); c is an integer from 1 to 5 (i.e. 1, 2, 3, 4 or 5); w is an integer from 1 to 5 (i.e. 1, 2, 3, 4 or 5); z is an integer from 5 to 10 (i.e. 5, 6, 7, 8, 9 or 10); Ra is unsubstituted C5-C15 straight chain alkyl (i.e. unsubstituted C5 straight chain alkyl, unsubstituted C6 straight chain alkyl, unsubstituted C7 straight chain alkyl, unsubstituted C8 straight chain alkyl, unsubstituted C9 straight chain alkyl, unsubstituted C10 straight chain alkyl, unsubstituted C11 straight chain alkyl, unsubstituted C12 straight chain alkyl, unsubstituted C13 straight chain alkyl, unsubstituted C14 straight chain alkyl, unsubstituted C15 straight chain alkyl); Rb is unsubstituted C5-C15 straight chain alkyl (i.e. unsubstituted C5 straight chain alkyl, unsubstituted C6 straight chain alkyl, unsubstituted C7 straight chain alkyl, unsubstituted C8 straight chain alkyl, unsubstituted C9 straight chain alkyl, unsubstituted C10 straight chain alkyl, unsubstituted C11 straight chain alkyl, unsubstituted C12 straight chain alkyl, unsubstituted C13 straight chain alkyl, unsubstituted C14 straight chain alkyl, unsubstituted C15 straight chain alkyl).

In some embodiments, the compound of formula A is selected from a compound of formula II, wherein a is an integer from 0 to 3 (i.e. 0, 1, 2 or 3); b is an integer from 4 to 6 (i.e. 4, 5 or 6); c is an integer from 1 to 3 (i.e. 1, 2 or 3); w is an integer from 1 to 3 (i.e. 1, 2 or 3); z is an integer from 5 to 8 (i.e. 5, 6, 7 or 8); Ra is unsubstituted C5-C13 straight chain alkyl (i.e. unsubstituted C5 straight chain alkyl, unsubstituted C6 straight chain alkyl, unsubstituted C7 straight chain alkyl, unsubstituted C8 straight chain alkyl, unsubstituted C9 straight chain alkyl, unsubstituted C10 straight chain alkyl, unsubstituted C11 straight chain alkyl, unsubstituted C12 straight chain alkyl, unsubstituted C13 straight chain alkyl); Rb is unsubstituted C9-C13 straight chain alkyl (i.e. unsubstituted C9 straight chain alkyl, unsubstituted C10 straight chain alkyl, unsubstituted C11 straight chain alkyl, unsubstituted C12 straight chain alkyl, unsubstituted C13 straight chain alkyl).

In some embodiments, the compound of formula A is selected from a compound of formula II, wherein a is1; b is 5; c is 1; w is 1; z is 6; Ra is unsubstituted C5-C13 straight chain alkyl (i.e. unsubstituted C5 straight chain alkyl, unsubstituted C6 straight chain alkyl, unsubstituted C7 straight chain alkyl, unsubstituted C8 straight chain alkyl, unsubstituted C9 straight chain alkyl, unsubstituted C10 straight chain alkyl, unsubstituted C11 straight chain alkyl, unsubstituted C12 straight chain alkyl, unsubstituted C13 straight chain alkyl; Rb is unsubstituted C10 straight chain alkyl, unsubstituted C11 straight chain alkyl, unsubstituted C12 straight chain alkyl.

In some embodiments, the compound of formula A is selected from a compound of formula II, the compound of formula II is selected from compound L0126, compound L0127 and compound L0128,

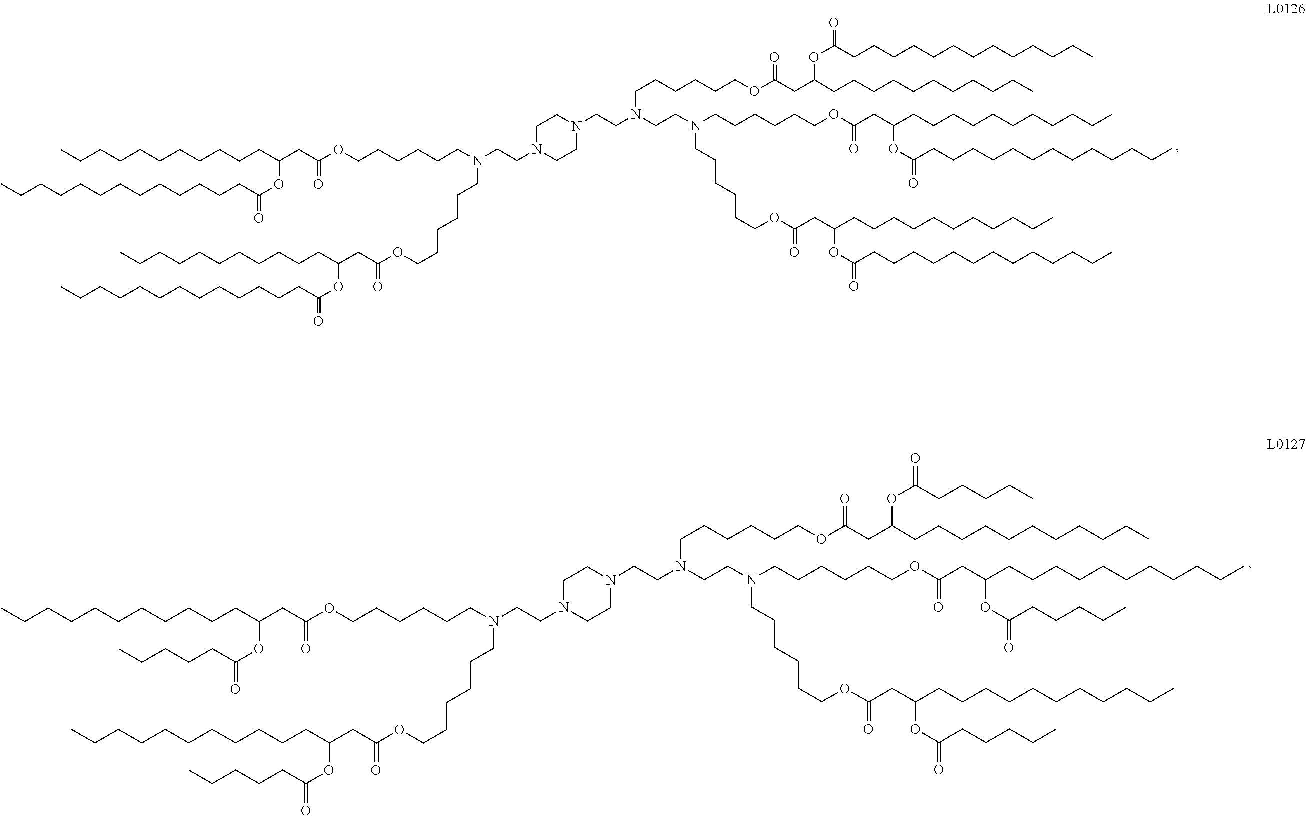

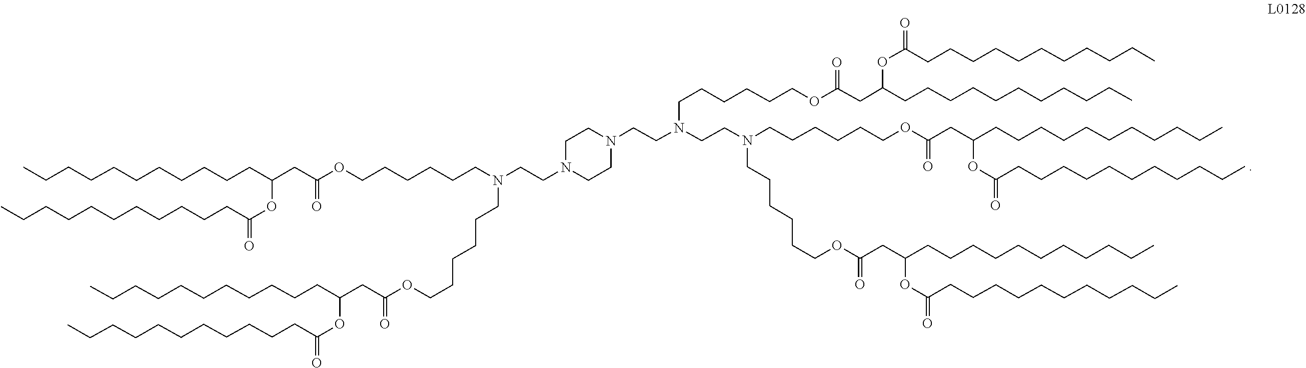 [SMILES: CCCCCCCCCCCC(=O)OC(CCCCCCCCCCC)CC(=O)OCCCCCCN(CCCCCCOC(=O)CC(CCCCCCCCCCC)OC(=O)CCCCCCCCCCC)CCN(CCCCCCOC(=O)CC(CCCCCCCCCCC)OC(=O)CCCCCCCCCCC)CCN1CCN(CCN(CCCCCCOC(=O)CC(CCCCCCCCCCC)OC(=O)CCCCCCCCCCC)CCCCCCOC(=O)CC(CCCCCCCCCCC)OC(=O)CCCCCCCCCCC)CC1]

In some embodiments, the compound of formula A is selected from a compound of formula III, wherein a, b and c are the same and are an integer from 1 to 9 (i.e. 1, 2, 3, 4, 5, 6, 7, 8 or 9).

In some embodiments, the compound of formula A is selected from a compound of formula III, wherein a, b and c are the same and are an integer from 1 to 5 (i.e. 1, 2, 3, 4 or 5).

In some embodiments, the compound of formula A is selected from a compound of formula III, wherein a, b and c are the same and are an integer from 1 to 5 (i.e. 1, 2, 3, 4 or 5); Ra is unsubstituted C5-C15 straight chain alkyl (i.e. unsubstituted C5 straight chain alkyl, unsubstituted C6 straight chain alkyl, unsubstituted C7 straight chain alkyl, unsubstituted C8 straight chain alkyl, unsubstituted C9 straight chain alkyl, unsubstituted C10 straight chain alkyl, unsubstituted C11 straight chain alkyl, unsubstituted C12 straight chain alkyl, unsubstituted C13 straight chain alkyl, unsubstituted C14 straight chain alkyl, unsubstituted C15 straight chain alkyl); Rb is unsubstituted C5-C15 straight chain alkyl (i.e. unsubstituted C5 straight chain alkyl, unsubstituted C6 straight chain alkyl, unsubstituted C7 straight chain alkyl, unsubstituted C8 straight chain alkyl, unsubstituted C9 straight chain alkyl, unsubstituted C10 straight chain alkyl, unsubstituted C11 straight chain alkyl, unsubstituted C12 straight chain alkyl, unsubstituted C13 straight chain alkyl, unsubstituted C14 straight chain alkyl, unsubstituted C15 straight chain alkyl).

In some embodiments, the compound of formula A is selected from a compound of formula III, wherein a, b and c are the same and are an integer from 3 to 5 (i.e. 3, 4 or 5); Ra is unsubstituted C5-C11 straight chain alkyl (i.e. unsubstituted C5 straight chain alkyl, unsubstituted C6 straight chain alkyl, unsubstituted C7 straight chain alkyl, unsubstituted C8 straight chain alkyl, unsubstituted C9 straight chain alkyl, unsubstituted C10 straight chain alkyl, unsubstituted C11 straight chain alkyl); Rb is unsubstituted C8-C13 straight chain alkyl (i.e. unsubstituted C8 straight chain alkyl, unsubstituted C9 straight chain alkyl, unsubstituted C10 straight chain alkyl, unsubstituted C11 straight chain alkyl, unsubstituted C12 straight chain alkyl, unsubstituted C13 straight chain alkyl).

In some embodiments, the compound of formula A is selected from a compound of formula III, wherein a, b and c are the same and are an integer from 3 to 5 (i.e. 3, 4 or 5); Ra is unsubstituted C5-C11 straight chain alkyl (i.e. unsubstituted C5 straight chain alkyl, unsubstituted C6 straight chain alkyl, unsubstituted C7 straight chain alkyl, unsubstituted C8 straight chain alkyl, unsubstituted C9 straight chain alkyl, unsubstituted C10 straight chain alkyl, unsubstituted C11 straight chain alkyl); Rb is unsubstituted C11 straight chain alkyl.

In some embodiments, the compound of formula A is selected from a compound of formula III, the compound of formula III is selected from compound L0129 and compound L0130,

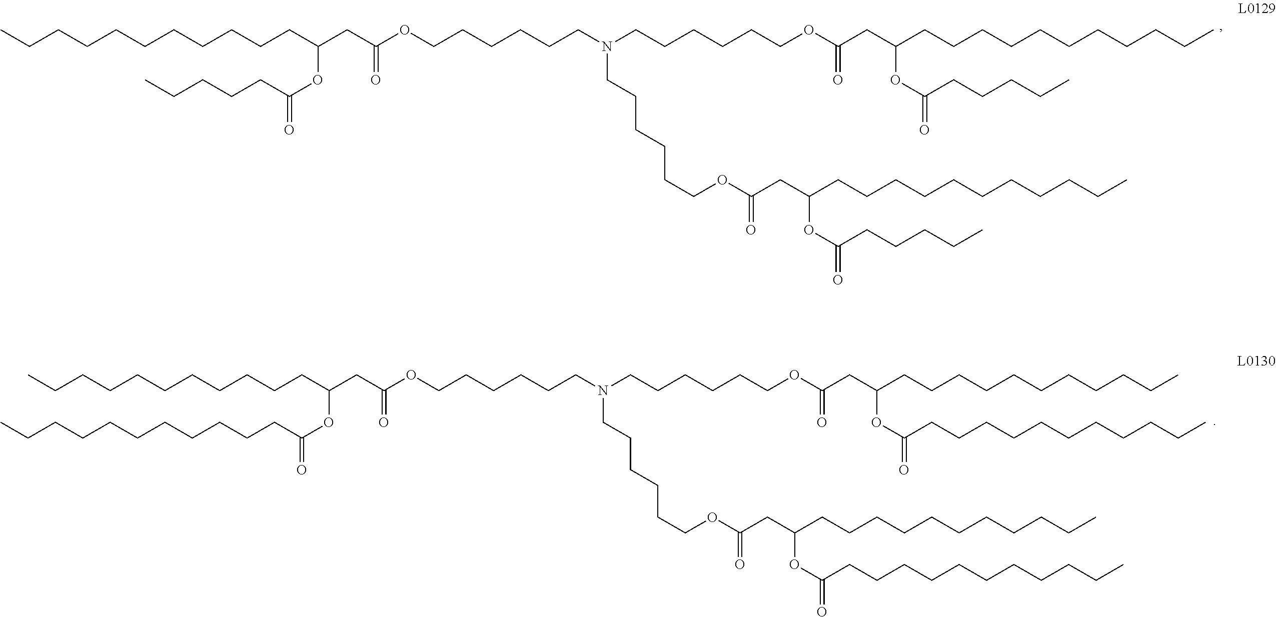

In some embodiments, the compound of formula A is selected from a compound of formula IV, wherein a is an integer from 0 to 5, b is an integer from 3 to 10, c is an integer from 3 to 10, z is an integer from 5 to 10, Ra is unsubstituted C5-C15 straight chain alkyl, Rb is unsubstituted C5-C15 straight chain alkyl.

In some embodiments, the compound of formula A is selected from a compound of formula IV, wherein a is an integer from 0 to 3, b is an integer from 4 to 6, c is an integer from 4 to 6, z is an integer from 5 to 8, Ra is unsubstituted C5-C13 straight chain alkyl, Rb is unsubstituted C9-C13 straight chain alkyl.

In some embodiments, the compound of formula A is selected from a compound of formula IV, wherein a is 1, b is 5, c is 5, z is 6, Ra is unsubstituted C5-C13 straight chain alkyl, Rb is unsubstituted C10-C15 straight chain alkyl.

In some embodiments, the compound of formula A is selected from a compound of formula IV, the compound of formula IV is selected from compound L0131,

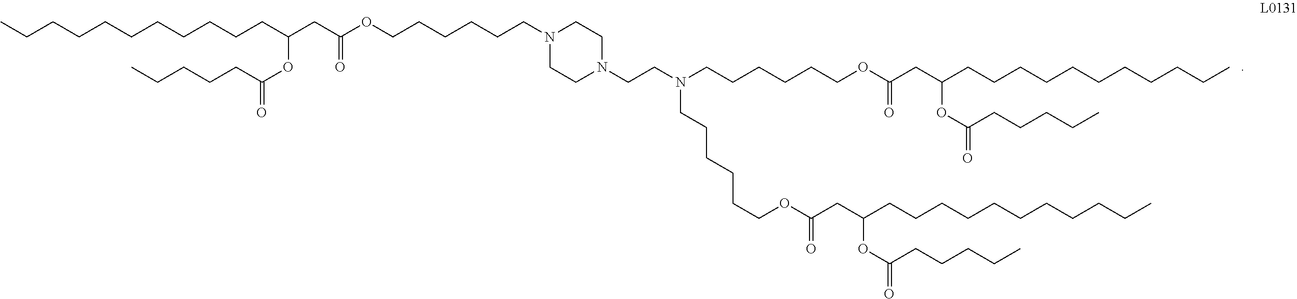 [SMILES: CCCCCCCCCCCC(CC(=O)OCCCCCCN(CCCCCCOC(=O)CC(CCCCCCCCCCC)OC(=O)CCCCC)CCN1CCN(CCCCCCOC(=O)CC(CCCCCCCCCCC)OC(=O)CCCCC)CC1)OC(=O)CCCCC]

In a second aspect, the present invention provides a lipid compound nanoparticle.

In some embodiments, the lipid compound nanoparticle comprises the following components: the compound of the first aspect and auxiliary materials.

In some embodiments, the lipid compound nanoparticle comprises the following components: the compound of the first aspect, nucleic acid and auxiliary materials.

In some embodiments, the auxiliary materials include at least one of a PEG derivative, a lipid, a lipid-like substance, an alcohol, a saccharide and an inorganic salt.

In some embodiments, the PEG derivative includes at least one of PEG modified phosphatidylethanolamine, PEG modified phosphatidic acid, PEG modified ceramide, PEG modified dialkylamine, PEG modified diacylglycerol, PEG modified dialkyglycerol, PEG modified stearic acid and PEG modified phosphatidylserine.

In some embodiments, the PEG derivative includes at least one of 1,2-dimyristoyl-sn-glycero methoxypolyethylene glycol, 1,2-distearoyl-sn-glycero-3-phosphoethanolamine-N -(amino (polyethylene glycol)), dilauroyl phosphatidylethanolamine-polyethylene glycol, dimyristoyl phosphatidylethanolamine-polyethylene glycol, dipalmitoyl phosphatidylcholine polyethylene glycol, dipalmitoyl phosphatidylethanolamine-polyethylene glycol, PEG-distearoyl glycerol, PEG-dipalmitoyl, PEG-dioleoyl, PEG-distearoyl, PEG-diacylglycerol amide, PEG-dipalmitoyl phosphatidylethanolamine and PEG-1,2-dimyristol oxypropyl-3-amine.

In some embodiments, the PEG derivative includes at least one of PEG-c-DOMG, PEG-c-DMA, PEG-DMG, PEG-DSG, PEG-DAG, PEG-DLPE, PEG-DPPC, ALC-0159, PEG-Mal, DSPE-PEG-Mal DMG-PEG2000, mPEG-DSPE, mPEG-STA, mPEG-PS, mPEG-DMPE and mPEG-DPPE.

In some embodiments, the lipid includes at least one of phospholipid and sterol.

In some embodiments, the phospholipid includes at least one of lecithin, 1,2-distearoyl -sn-glycero-3-phosphocholine, 1,2-dioleoyl-sn-glycero-3-phosphoethanolamine, 1,2-dioleoyl-sn -glycero-3-phosphocholine, 1,2-dimyristoyl-sn-glycero-3-phosphocholine, 1,2-dioleoyl-sn -glycero-3-phosphocholine, 1,2-dipalmitoyl-sn-glycero-3-phosphocholine, 1,2-diundecanoyl-sn -glycero-phosphocholine, 1-palmitoyl-2-oleoyl-sn-glycero-3-phosphocholine, Dilauroylphosphatidylcholine (DLPC), 1,2-Diheneicosanoyl-sn-glycero-3-phosphocholine (DUPC), 1-Palmitoyl-2-oleoyl-sn-glycero-3-phosphocholine (POPC), 1,2-Diisooctyl-OT -glycero-3-phosphocholine (18:0 Diether PC), 1-Oleoyl-2-cholesterylhemisuccinoyl-OT-glycero -S-phosphocholine (OChemSPC), 1,2-Dilinolenoyl-sn-glycero-3-phosphocholine, 1,2-Diarachidonoyl-sn-glycero-3-phosphocholine, 1,2-Didocosahexaenoyl-sn-glycero-3-phosphocholine, 1,2-Diphenylglyceryl-sn-glycero-3-phosphoethanolamine (ME16.0 PE), 1,2-Distearoyl-sn-glycero-3-phosphoethanolamine, 1,2-Dioctanoyl-sn-glycero-3-phosphoethanolamine, 1,2-Dilinoleoyl-sn-glycero-3-phosphoethanolamine, 1,2-Diarachidonoyl -sn-glycero-3-phosphoethanolamine, 1,2-Didocosahexaenoyl-sn-glycero-3-phosphoethanolamine, 1,2-Dioleoyl-sn-glycero-3-phospho-L-glycerol Sodium Salt (DOPG), Monopalmitoyl Phosphatidylcholine (MPPC), Myristoyl Stearoyl Phosphatidylcholine (MSPC), 1-palmitoyl-2-myristoyl-sn-glycero-3-phosphocholine (PMPC), 1-Palmitoyl-2-stearoyl-sn-glycero-3-phosphocholine (PSPC), 1-stearoyl-2-myristoyl-sn-glycero-3-phosphocholine (SMPC), 1-Stearoyl-2-palmitoyl-sn-glycero-3-phosphocholine (SPPC), 2-Oleoyl-1-stearoyl-sn-glycero-3-phosphocholine (SOPC), 1,2-Ditetradecyl-rac-glycero-3-phosphoethanolamine (DMPE), 1,2-Distearoyl-sn-glycero-3-phosphorylethanolamine (DSPE), and Sphingomyelin.

In some embodiments, the sterol includes at least one of cholesterol, lanosterol, 5 alpha-cholestan-3 β-alcohol, coprosterol, sitosterol, ergosterol, campesterol, stigmasterol, brassicasterol, tomatidine, ursolic acid and α-tocopherol.

In some embodiments, the lipid compound nanoparticle includes the compound of the first aspect, a PEG derivative and a lipid; the lipid includes at least one of phospholipid and sterol.

In some embodiments, calculated on the total molar amount of each component of the lipid compound nanoparticle, the compound of the first aspect has an amount of 14.8 mol %-70.0 mol %.

In some embodiments, calculated on the total molar amount of each component of the lipid compound nanoparticle, the PEG derivative has an amount of 0.4 mol %-10 mol %.

In some embodiments, calculated on the total molar amount of each component of the lipid compound nanoparticle, the phospholipid has an amount of 5.0 mol %-50.0 mol %.

In some embodiments, calculated on the total molar amount of each component of the lipid compound nanoparticle, the sterol has an amount of or 10.0 mol %-75.0 mol%. In some embodiments, calculated on the total molar amount of each component of the lipid compound nanoparticle, the sterol has an amount of or 15.0 mol %-75.0 mol%.

In some embodiments, a molar ratio of PEG derivative:phospholipid:sterol: the compound of the first aspect is (0.4-10.0):(5.0-50.0):(10.0-75.0):(14.8-70.0). In some embodiments, a molar ratio of PEG derivative:phospholipid:sterol: the compound of the first aspect is (0.4-10.0):(5.0-40.0):(15.0-75.0):(14.8-70.0).

In some embodiments, a molar ratio of PEG derivative:phospholipid:sterol: the compound of the first aspect is 2.50:16.00:16.50:65.00, 1.00:5.00:64.00:30.00, 0.40:8.00:56.60:35.00, 1.00:8.00:61.00:30.00, 1.20:12.00:38.30:48.50, 1.70:9.00:40.80:48.50, 2.50:16.00:21.50:60.00, 1.20:9.00:47.80:42.00, 1.00:16.00:34.50:48.50, 2.50:8.00:41.00:48.50, 1.50:8.00:25.50:65.00, 2.50:11.50:51.00:35.00, 1.50:8.00:30.50:60.00, 1.00:5.00:62.00:32.00, 0.60:5.00:54.40:40.00, 1.50:16.00:47.50:35.00, 1.63:12.99:45.38:40.00, 1.20:8.00:55.8:35.00, 1.30:8.00:55.70:35.00, 0.40:5.00:59.60:35.00, 1.00:20.00:37.00:42.00, 1.00:25.00:34.00:40.00, 3.20:12.00:29.80:55.00, 1.00:8.00:53.00:38.00, 2.50:9.50:33.00:55.00, 10.00:16.00:54.00:20.00, 3.00:30.00:42.00:25.00, 3.20:16.80:10.00:70.00, 3.00:17.00:25.00:55.00, 3.20:16.80:15.00:65.00, 4.20:11.00:70.00:14.80, 6.20:6.80:75.00:15.00, 1.50:11.50:38.50:48.50, 7.50:9.61:35.56:47.33, 3.00:9.50:32.50:55.00, 0.95:7.58:26.47:65.00, 1.40:11.15:38.95:48.50, 2.10:7.98:47.90:42.02, 3.00:6.00:43.00:48.00, 1.60:30.00:30.00:40.00, 1.60:35.00:35.00:30.00, 1.60:40.00:40.00:20.00, or 1.80:40.00:27.20:31.00.

In some embodiments, a molar ratio of PEG derivative:phospholipid:sterol: the compound of the first aspect is (0.4-1.5):(8.0-11.5):(38.5-56.6):(35.0-48.5).

In some embodiments, a molar ratio of PEG derivative:phospholipid:sterol: the compound of the first aspect is 0.40:8.00:56.60:35.00, 1.50:11.50:38.50:48.50 or 1.40:11.15:38.95:48.50.

In a third aspect, the present invention provides a nucleic acid nanoparticle complex.

A nucleic acid nanoparticle complex comprises a nucleic acid and at least one lipid compound nanoparticle of the second aspect.

In some embodiments, a molar ratio of ionizable nitrogen atoms of the compound to phosphorus atoms of the nucleic acid in the nucleic acid nanoparticle complex is 5-50. In some embodiments, a molar ratio of ionizable nitrogen atoms of the compound to phosphorus atoms of the nucleic acid in the nucleic acid nanoparticle complex is 5, 6, 7, 8, 9, 10, 15, 19, 20, 25, 30, 35, 40, 45 or 50.

In some embodiments, the nucleic acid comprises: deoxyribonucleic acid (DNA) or ribonucleic acid (RNA). RNA includes but is not limited to small interfering RNA (siRNA), asymmetric interfering RNA (aiRNA), microRNA (miRNA), Dicer substrate RNA (dsRNA), self replicating RNA (saRNA), small hairpin RNA (shRNA), circular RNA (circRNA), messenger RNA (mRNA) and their combinations. mRNA may be synthesized using various known methods. For example, mRNA may be synthesized through in vitro transcription (IVT). The specific conditions vary depending on the specific application.

In a fourth aspect, the present invention provides a pharmaceutical composition.

A pharmaceutical composition comprises the lipid compound nanoparticle of the second aspect or the nucleic acid nanoparticle complex of the third aspect, and a pharmaceutically acceptable adjuvant.

The formulation of the pharmaceutical composition may be injection, suppository, eye drop, tablet, capsule, suspension or inhalant, etc.

In some embodiments, the pharmaceutical composition comprises at least one RNA for treating or preventing diseases. The composition containing RNA contains at least coding RNA and non coding RNA; the coding RNA comprises at least one coding region encoding at least one therapeutic protein or peptide and immunogenic protein or peptide; The encoded RNA is mRNA.

The therapeutic proteins or peptides may be cytokines, chemokines, suicide gene products, immunogenic proteins or peptides, apoptosis inducers, angiogenesis inhibitors, heat shock proteins, tumor antigens, β-catenin inhibitors, STING pathway activators, checkpoint modulators, innate immune activators, antibodies, dominant negative receptors and decoy receptors, myeloid-derived suppressor cell (MDSCs) inhibitors, IDO pathway inhibitors, and proteins or peptides that bind to apoptosis inhibitors.

The immunogenic protein or peptide may be a full-length sequence or a partial sequence of at least one protein or peptide of one of the following viruses or bacteria: a novel coronavirus (SARS-COV-2); a Human Papilloma Virus (HPV); an influenza A or B virus or any other orthomyxovirus (influenza C virus); picornaviruses, such as rhinovirus or hepatitis A virus; togaviruses, such as α viruses or rubella viruses, e.g., sindbis virus, semliki forest virus, or measles virus; rubella virus; coronaviruses, in particular of SARS-COV-2, HCV-229E or HCV-OC43 subtypes; rhabdoviruses, such as rabies virus; paramyxoviruses such as mumps virus; reoviruses, such as group A, B or C rotavirus; hepadnaviruses, such as hepatitis B virus; papovaviruses, such as human papilloma virus of any serotype; adenoviruses, types 1 to 47 in particular; herpes viruses, such as herpes simplex virus 1, 2 or 3; cytomegalovirus, preferably CMVpp 65; EB virus; vaccinia virus; the bacterium Chlamydophila pneumoniae; flaviviruses such as dengue virus types 1 to 4, yellow fever virus, west nile virus, japanese encephalitis virus; hepatitis C virus; calicivirus; filoviruses, such as ebola virus; borna virus; bunyavirus, such as rift valley fever virus; arenaviruses such as lymphocytic choriomeningitis virus or hemorrhagic fever virus; retroviruses, such as HIV; parvovirus.

In a fifth aspect, the present invention provides use of the compound of the first aspect or the lipid compound nanoparticle of the second aspect or the nucleic acid nanoparticle complex of the third aspect or the pharmaceutical composition of the fourth aspect in the manufacture of a product for delivering nucleic acids in vivo.

Use of the compound of the first aspect or the lipid compound nanoparticle of the second aspect or the nucleic acid nanoparticle complex of the third aspect or the pharmaceutical composition of the fourth aspect in the manufacture of a product for delivering nucleic acids in vivo.

The invention provides a ribonucleic acid vaccine which can safely induce a specific immune system naturally existing in an organism to generate almost any target protein or fragment thereof, wherein the ribonucleic acid vaccine takes RNA (such as messenger RNA (mRNA)) as a core and takes the nanoparticles of the second aspect as a delivery vector, and the ribonucleic acid vaccine comprises infectious pathogen vaccines of bacteria, viruses and the like and tumor vaccines. In some embodiments, RNA is modified. The RNA vaccines disclosed herein can be used to induce an immune response against an infectious agent or cancer, including cellular and humoral immune responses, without risk that could lead to insertional mutagenesis, for example. The RNA vaccines using the nanoparticles of the second aspect as the delivery vehicle can be used in a variety of settings, depending on the infectious agent and the incidence of cancer. The RNA vaccines can be used for the prevention and/or treatment of infectious agents or cancers at various metastatic stages or degrees. The RNA vaccines using the nanoparticle of the second aspect as the delivery vector have superior properties, due to the selective transfection of DC cells, and higher transfection efficiency and transfection expression level can be achieved, and higher antibody titer can be generated, when the transfection rate is the same or lower.

The present invention provides a ribonucleic acid (RNA) vaccine that is constructed based on the knowledge that RNA (e.g., messenger RNA (mrna)) can safely direct the cellular machinery of the body to produce almost any protein of interest, from natural proteins to antibodies and other entirely novel proteins that can have therapeutic activity both inside and outside the cell. RNA (e.g., mRNA) vaccines can be used in various situation according to the prevalence of infection or the degree or level of unmet medical need.

The lipid compound nanoparticles of the second aspect of the invention or the nanoparticle complexes of the third aspect of the invention are useful for the prevention, treatment and/or amelioration of a disease selected from the group consisting of: cancer or tumor diseases, infectious diseases (such as viral, bacterial or protozoalinfectious diseases), autoimmune diseases, allergies or allergic diseases, monogenic diseases, i.e. (genetic) diseases, or genetic diseases in general, diseases which have a genetic background and are typically caused by a defined genetic defect and inherited according to Mendel's rules, cardiovascular diseases, neuronal diseases, respiratory diseases, digestive diseases, skin diseases, musculoskeletal disorders, connective tissue disorders, neoplasms, immunodeficiency, endocrine, nutritional and metabolic diseases, eye and ear diseases.

The nucleic acid vaccines of the present invention can be administered by any route which results in a therapeutically effective outcome. These include, but are not limited to, intradermal, hypodermic, intraperitoneal, oral, intramuscular, intranasal, intraocular, upper respiratory, intravenous, vaginal, or rectal administration. In some embodiments, the nucleic acid vaccines of the present invention are administered by injections.

### ADVANTAGEOUS EFFECTS

Compared to the prior art, the invention has the following technical effects:

The compound, lipid compound nanoparticle or nucleic acid nanoparticle complex provided herein has the advantages of good biocompatibility, high transfection efficiency, low toxicity and excellent technical effects.

### BRIEF DESCRIPTION OF DRAWINGS

FIG. 1 shows a 1H NMR chart of compound L0111.

FIG. 2 shows a 1H NMR chart of compound L0112.

FIG. 3 shows a 1H NMR chart of compound L0113.

FIG. 4 shows a 1H NMR chart of compound L0114.

FIG. 5 shows a 1H NMR chart of compound L0115.

FIG. 6 shows a 1H NMR chart of compound L0116.

FIG. 7 shows a 1H NMR chart of compound L0117.

FIG. 8 shows a 1H NMR chart of compound L0118.

FIG. 9 shows a 1H NMR chart of compound L0119.

FIG. 10 shows a 1H NMR chart of compound L0120.

FIG. 11 shows a 1H NMR chart of compound L0121.

FIG. 12 shows a 1H NMR chart of compound L0122.

FIG. 13 shows a 1H NMR chart of compound L0123.

FIG. 14 shows a 1H NMR chart of compound L0124.

FIG. 15 shows a 1H NMR chart of compound L0125.

FIG. 16 shows a 1H NMR chart of compound L0126.

FIG. 17 shows a 1H NMR chart of compound L0127.

FIG. 18 shows a 1H NMR chart of compound L0128.

FIG. 19 shows a 1H NMR chart of compound L0129.

FIG. 20 shows a 1H NMR chart of compound L0130.

FIG. 21 shows a 1H NMR chart of compound L0131.

FIG. 22 shows a mass spectral profile of compound L0132.

FIG. 23 shows a 1H NMR chart of compound L0133.

FIG. 24 shows a 1H NMR chart of compound L0134.

FIG. 25 shows a 1H NMR chart of compound L0135.

FIG. 26 shows a 1H NMR chart of compound L0136.

FIG. 27 shows a statistical plot of the silencing efficiency of a nucleic acid nanoparticle complex loaded with EGFP-siRNA delivered to Hela-EGFP cells in example 9.

FIG. 28 shows the expression of luciferase in mice after intravenous injection of a nucleic acid nanoparticle complex loaded with FLuc mRNA detected by IVIS in example 10.

FIG. 29 shows the expression of luciferase in mice after intraperitoneal injection of a nucleic acid nanoparticle complex loaded with FLuc mRNA detected by IVIS in example 10.

FIG. 30 shows the expression of luciferase in mice after intramuscular injection of a nucleic acid nanoparticle complex loaded with FLuc mRNA detected by IVIS in example 10.

FIG. 31 shows the expression of luciferase in mice after hypodermic injection of a nucleic acid nanoparticle complex loaded with FLuc mRNA detected by IVIS in example 10.

FIG. 32 is a statistical plot of serum IgG antibody levels of mice immunized with the corona S-mRNA-loaded nucleic acid nanoparticle complexes of example 11; the horizontal axis represents the 14 th and 28 th days after the first immunization of various formulations, and the vertical axis represents the difference in OD values of optical density between two wavelengths, OD value is an index for determining IgG antibody level in serum and reflects the level of anti S protein IgG in serum. Intravenous injection: IV, intramuscular injection: IM, hypodermic injection: H.

FIG. 33 shows a 1H NMR chart of compound L0137.

FIG. 34 shows a 1H NMR chart of compound L0138.

FIG. 35 shows a 1H NMR chart of compound L0139.

FIG. 36 shows a 1H NMR chart of compound L0140.

FIG. 37 shows the expression of luciferase in mice after intramuscular injection of a nucleic acid nanoparticle complex loaded with FLuc mRNA detected by IVIS in example 10.

FIG. 38 shows the expression of luciferase in mice after intravenous injection of a nucleic acid nanoparticle complex loaded with FLuc mRNA detected by IVIS in example 10.

FIG. 39 shows the expression of luciferase at the injection site in mice after intramuscular injection of a nucleic acid nanoparticle complex loaded with FLuc mRNA detected by IVIS in example 10.

FIG. 40 shows the expression of luciferase in liver of mice after intramuscular injection of a nucleic acid nanoparticle complex loaded with FLuc mRNA detected by IVIS in example 10.

FIG. 41 shows the expression of luciferase in spleen of mice after intramuscular injection of a nucleic acid nanoparticle complex loaded with FLuc mRNA detected by IVIS in example 10.

FIG. 42 shows the expression of luciferase in lymph nodes of mice after intramuscular injection of a nucleic acid nanoparticle complex loaded with FLuc mRNA detected by IVIS in example 10.

FIG. 43 shows the percentage of liver targeting after intramuscular injection of a nucleic acid nanoparticle complex loaded with FLuc mRNA detected by IVIS in example 10.

FIG. 44 shows the percentage of spleen targeting after intramuscular injection of a nucleic acid nanoparticle complex loaded with FLuc mRNA detected by IVIS in example 10.

FIG. 45 shows the ratio of radiance in liver/LNs after intramuscular injection of a nucleic acid nanoparticle complex loaded with FLuc mRNA detected by IVIS in example 10.

FIG. 46 shows the ratio of radiance in liver/spleens after intramuscular injection of a nucleic acid nanoparticle complex loaded with FLuc mRNA detected by IVIS in example 10.

FIG. 47 shows the expression of luciferase in mice after intravenous injection of a nucleic acid nanoparticle complex loaded with FLuc mRNA detected by IVIS in example 10.

FIG. 48 shows the expression of luciferase in liver of mice after intravenous injection of a nucleic acid nanoparticle complex loaded with FLuc mRNA detected by IVIS in example 10.

FIG. 49 shows the expression of luciferase in spleen of mice after intravenous injection of a nucleic acid nanoparticle complex loaded with FLuc mRNA detected by IVIS in example 10.

FIG. 50 shows the percentage of liver targeting after intravenous injection of a nucleic acid nanoparticle complex loaded with FLuc mRNA detected by IVIS in example 10.

FIG. 51 shows the percentage of spleen targeting after intravenous injection of a nucleic acid nanoparticle complex loaded with FLuc mRNA detected by IVIS in example 10.

FIG. 52 shows the ratio of radiance in liver/spleens after intravenous injection of a nucleic acid nanoparticle complex loaded with FLuc mRNA detected by IVIS in example 10.

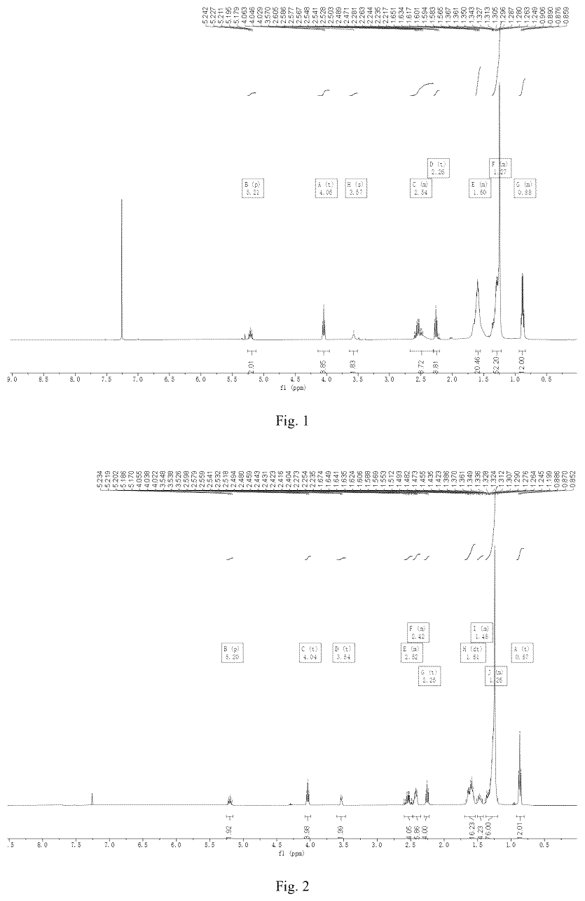

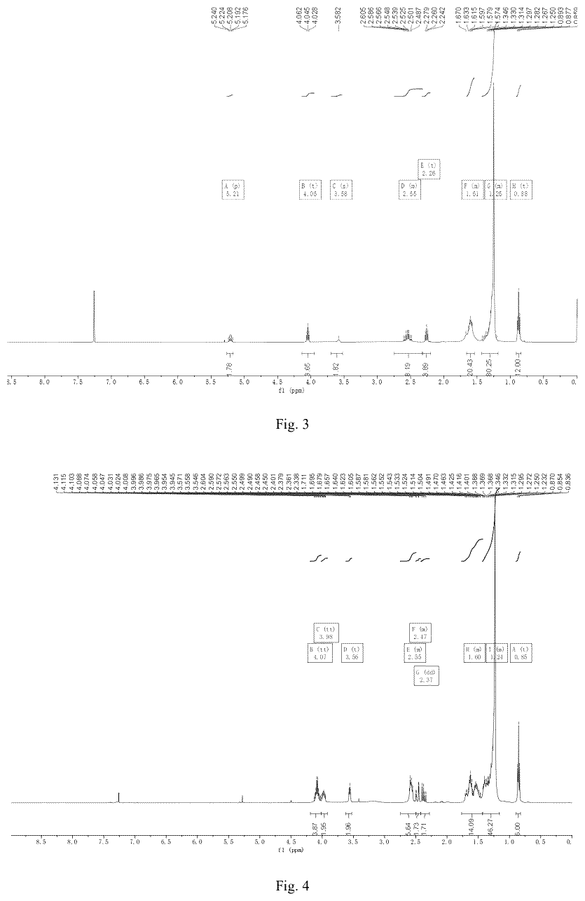

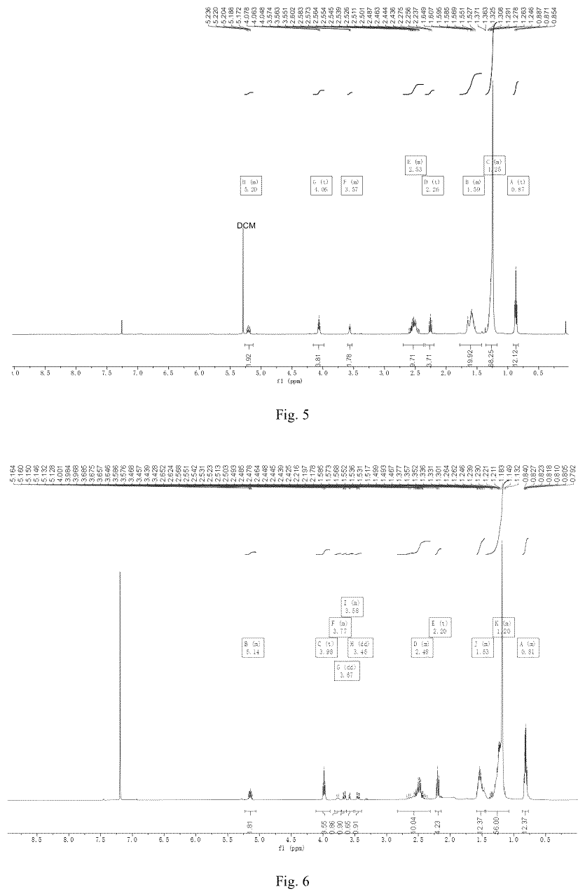

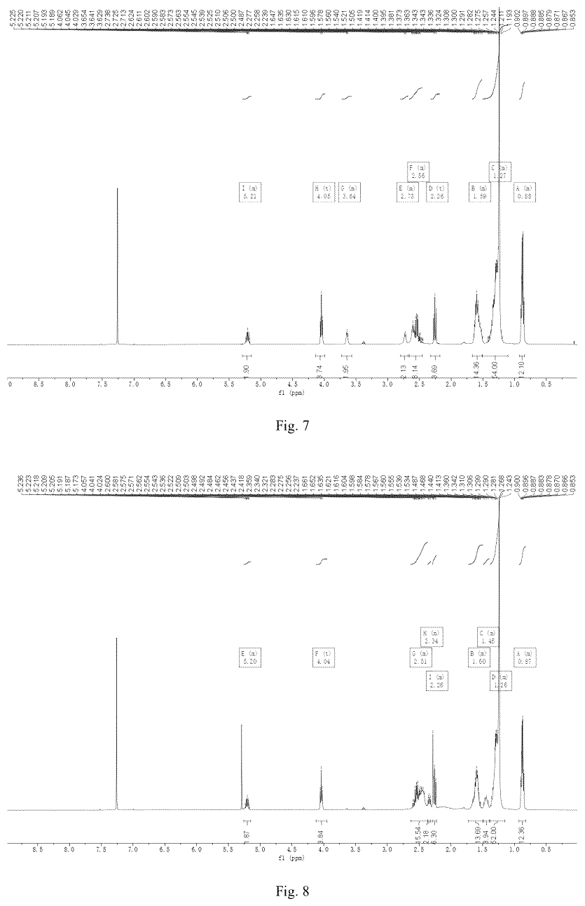

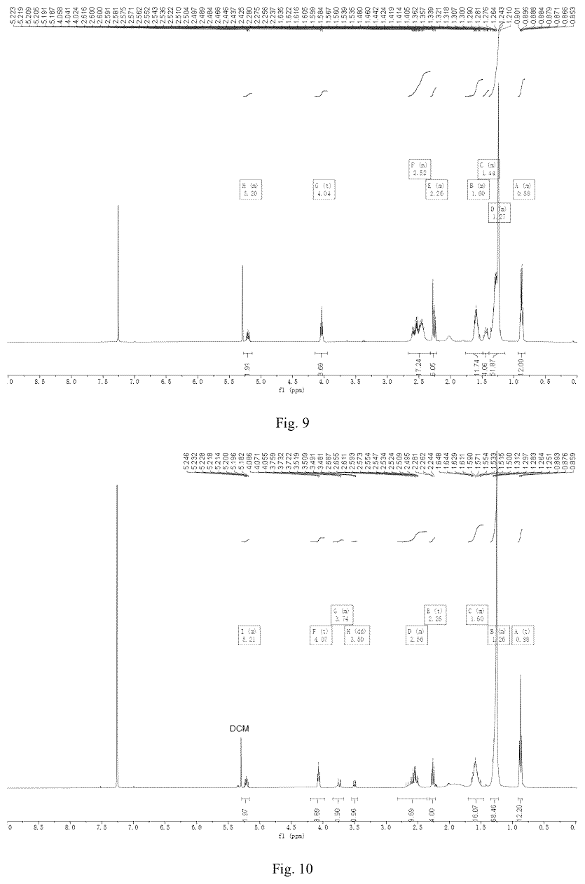

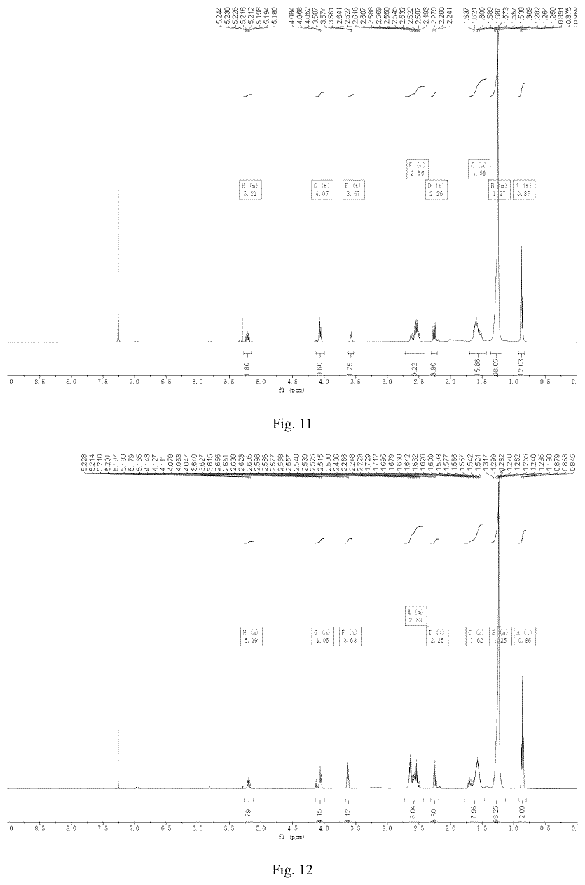

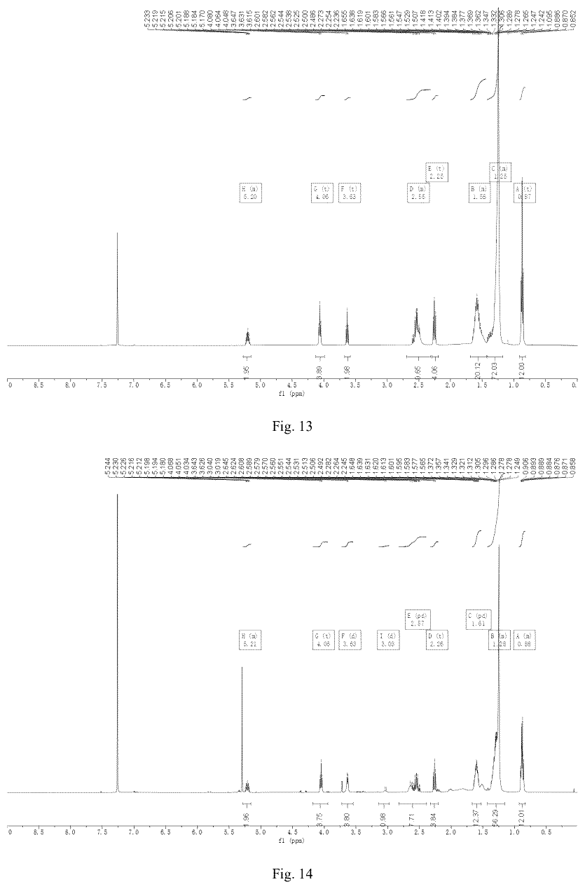

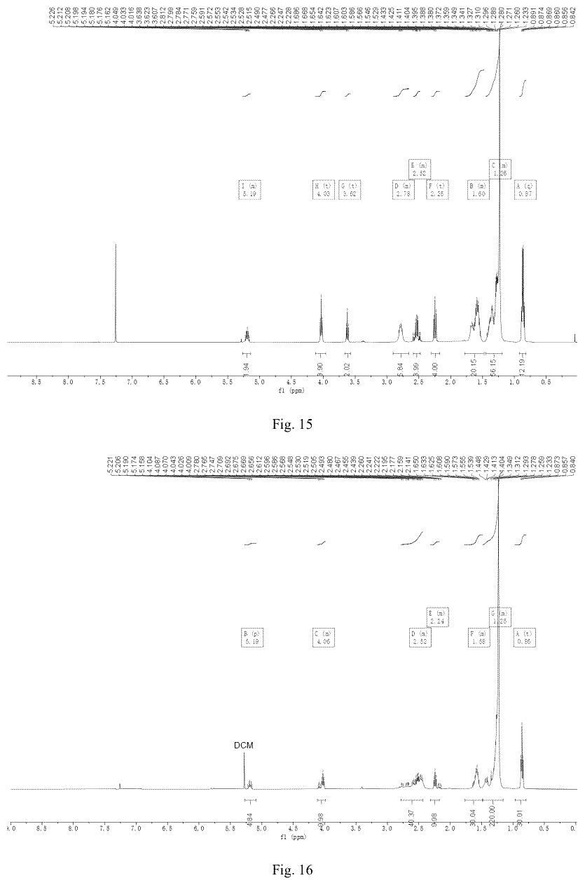

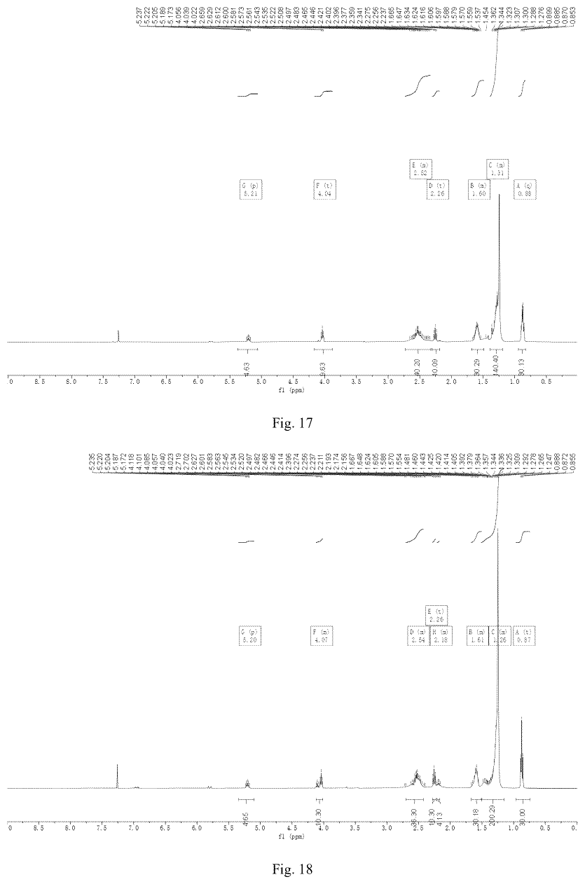

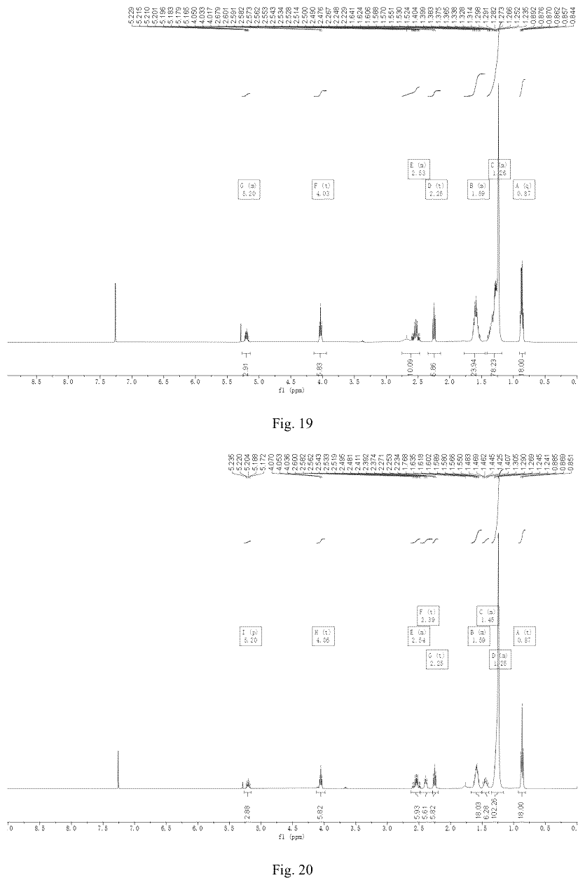

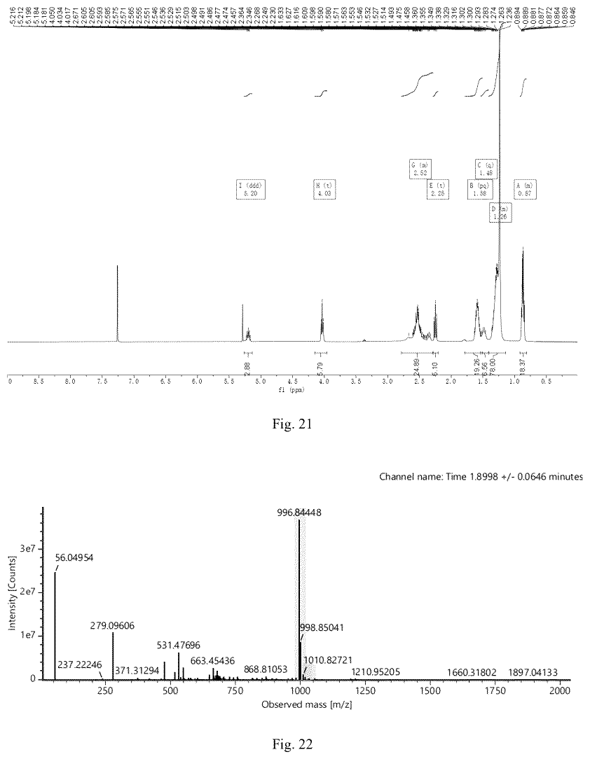

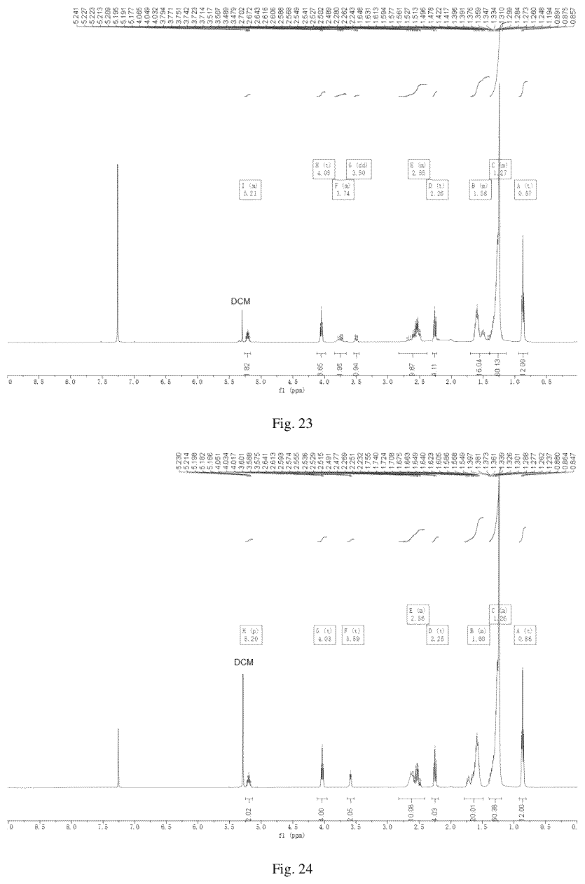

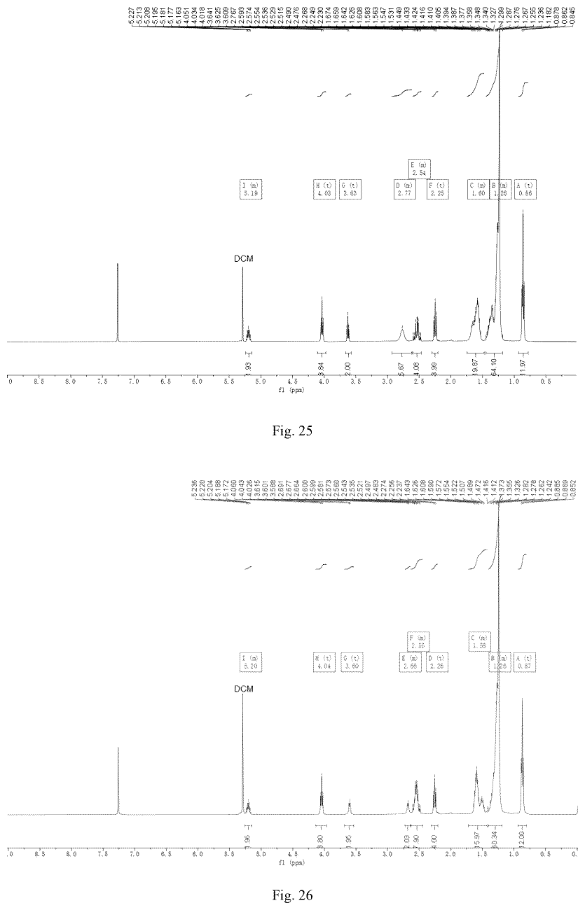

### DEFINITION

Unless otherwise indicated, as used herein, the following terms and phrases are intended to have the following meanings:

“Compound of the invention” refers to a compound of formula I or pharmaceutically acceptable salts, tautomers, polymorphs, isomers and solvates thereof. Likewise, the phrase “compound of formula I” refers to the compound of the formula and pharmaceutically acceptable salts, tautomers, polymorphs, isomers and solvates thereof.

In the present invention, the expressions “compound I” and “compound of formula I” refer to the same compound.

“V/V” represents a volume ratio.

IC₅₀ represents the half inhibitory concentration.

The term “plurality” means at least 2, such as 2, 3, 4, or 5, etc.

In the present invention, atoms in a compound structure have the following meaning: F represents fluorine, Cl represents chlorine, and D represents deuterium.

The term “and/or” should be understood to mean any one of the options or a combination of any two or more of the options.

The term “optional” or “optionally” means that the subsequently described event or circumstance may, but need not, occur.

“Room temperature” in the present invention refers to ambient temperature, ranging from about 10° C. to about 40° C. In some embodiments, “room temperature” refers to a temperature of from about 20° C. to about 30° C.; in other embodiments, “room temperature” refers to a temperature of from about 25° C. to about 30° C.; in still other embodiments, “room temperature” refers to 10° C., 15° C., 20° C., 25° C., 30° C., 35° C., 40° C., etc.

Term “subject” refers to any animal, including mammals such as mice, rats, other rodents, rabbits, dogs, cats, swine, cattle, sheep, horses, primates, and humans. In some embodiments, the term refers to a subject, particularly a mammalian subject, for whom diagnosis, prognosis, or therapy is desired or needed. In some embodiments, the subject is a human.

“Alkyl” is a hydrocarbon containing a normal carbon atom, a secondary carbon atom, a tertiary carbon atom, or a ring carbon atom. For example, an alkyl group may have 1 to 20 carbon atoms (i.e., C₁-C₂₀ alkyl), 1 to 10 carbon atoms (i.e., C₁-C₁₀ alkyl), 1 to 8 carbon atoms (i.e., C₁-C₈ alkyl), or 1 to 6 carbon atoms (i.e., C₁-C₆ alkyl). Examples of suitable alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃ or octyl (—(CH₂)₇CH₃).

In the terms “j-k”, “j-k member” or “Cj-Ck”, j and k are each independently any non-zero natural number, and k>j; for example, “1-4” means 1, 2, 3 or 4, “4-6” means 4, 5 or 6; “C3-C6” means C3, C4, C5 or C6. And so on.

In the nucleic acid nanoparticle complex, the positive charge is typically provided by ionizable nitrogen (N) in ionizable lipids (such as the compound of formula A provided herein), and the negative charge is provided by phosphate (P) in the nucleic acid molecule, which can be held together by electrostatic adsorption. The nitrogen to phosphorus ratio (N/P) is the ratio of the number of moles of ionizable N in the ionizable lipid to the number of moles of P in the nucleic acid molecule.

### DETAILED DESCRIPTION

In order to make the technical solutions of the present invention better understood by those skilled in the art, some non-limiting examples are further disclosed below to further explain the present invention in detail.

The reagents used in this invention can be purchased from the market or prepared using the methods described in this invention.

The term “×g” represents the centrifugal acceleration at how many times the gravitational acceleration, for example, “5000×g” represents the centrifugal acceleration at 5000 times the gravitational acceleration.

DMG-PEG2000 represents 1,2-dimyristoyl-sn-glycerol methoxy polyethylene glycol 2000; PEG-DMPE represents diglycidyl phosphatidylethanolamine polyethylene glycol; PEG-DPPC represents dipalmitoyl phosphatidylcholine polyethylene glycol; MPEG-STA represents methoxy polyethylene glycol monostearate; MPEG-PS represents methoxy polyethylene glycol phosphatidylserine; MPEG DPPE represents dipalmitoyl phosphatidylethanolamine methoxy polyethylene glycol; MPEG-DSPE represents methoxy polyethylene glycol phosphatidylethanolamine; MPEG-DMPE represents Methoxypolyethylene glycol 1,2-tetradecanoyl phosphatidylethanolamine; DOTAP represents (2,3-dioloyl-propyl) -trimethylamine sulfate; DOPE represents 1,2-dioleoyl-sn-glycerol-3-phosphateethanolamine; DSPC represents 1,2-distearoyl-sn-glycerol-3-phosphocholine; Chol represents cholesterol; DMPC represents 1,2-dimyristoyl-sn-glycerol-phosphocholine; PC represents lecithin; Tween®20 represents Tween 20; DPPC represents 1,2-dipalmitoyl-sn-glycerol-3-phosphocholine; Span®80 represents span 80. EDCI represents 1-(3-dimethylaminopropyl)-3-ethylcarbodiimide hydrochloride. DMAP represents 4-dimethylaminopyridine.

L0111 represents compound L0111, L0112 represents compound L0112, L0113 represents compound L0113, and so on.

FLuc-mRNA represents the messenger RNA encoding firefly luciferase; EGFP-pDNA represents plasmid encoding green fluorescent protein; EGFP-siRNA represents small interfering RNA silencing expression of enhanced green fluorescent protein genes; S-mRNA or Spike-mRNA represents the messenger RNA encoding S protein.

FLuc-mRNA manufacturer: Hongene Biotech Corporation.

The specific information of FLuc mRNA stock solution:
Product name: FLuc-mRNA (N1-Me-pseudo U);
Product description: 1939 nucleotides in length;
Modifications: Fully substituted with N1-Me-pseudo UTP;
Concentration: 1.0 mg/mL;
Storage condition: 1 mM sodium citrate with pH 6.4;
Storage requirements: −40° C. or below.

### EXAMPLE 1: PREPARATION OF COMPOUND L0111

The cationic lipid compounds of the present invention are prepared by any previously known synthetic method known to those of ordinary skill in the art. The raw materials including compound 1, compound 2, compound 3 and compound 4 in the preparation method can be purchased commercially or synthesized by a conventional method.

The simple synthesis method and the specific preparation process of compound L0111 are described as follows:

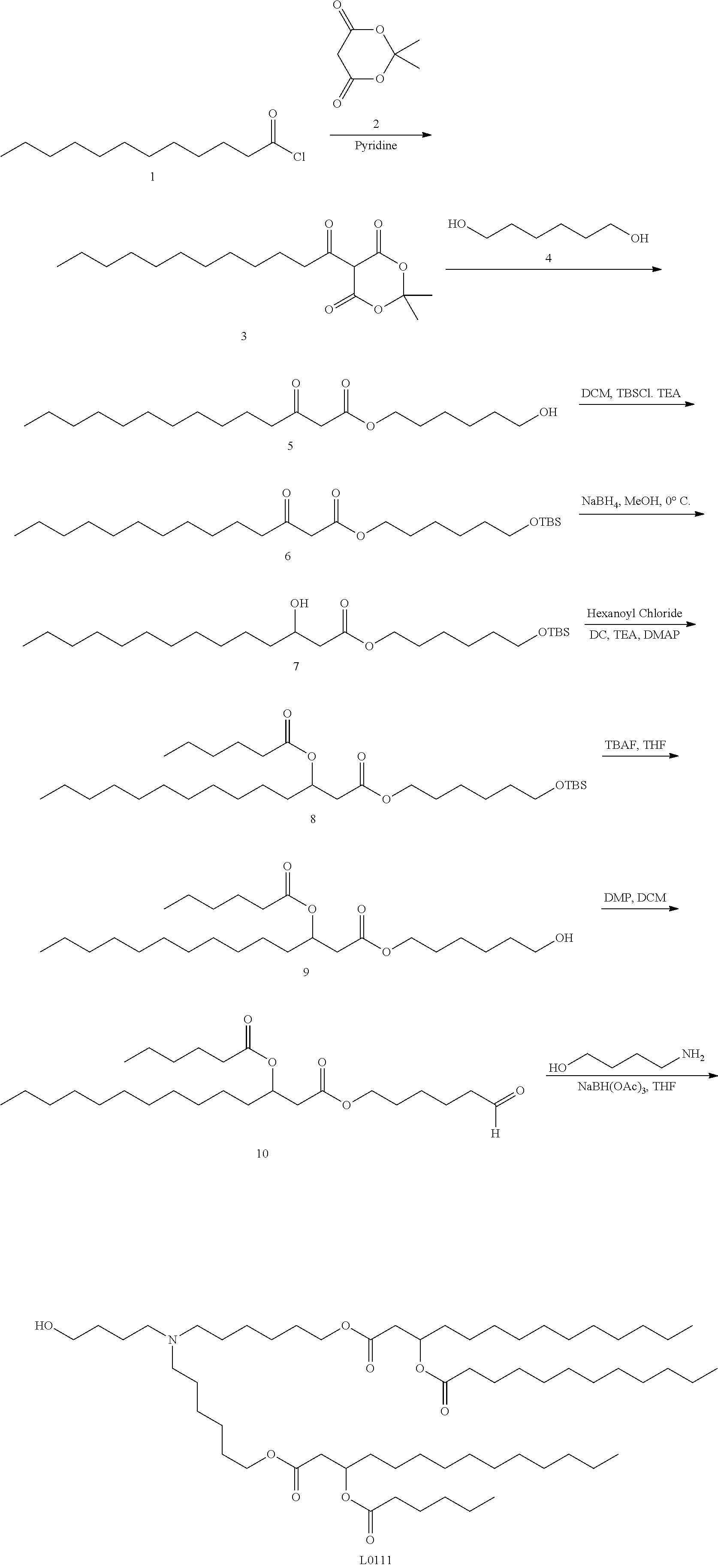

Synthesis of Compound 3: 5.766 g of 2,2-dimethyl-1,3-dioxane-4,6-dione (compound 2) and 10.200 mL of lauroyl chloride (compound 1) were mixed with 30 mL of ultradry DCM, and the mixture was stirred for 5 min in an ice bath; then 6.430 mL of pyridine was added, followed by addition of 16 mL of ultradry DCM, and the mixture was stirred for 1 h in an ice bath; after removing the ice bath, the reaction was continued, and consumption of 2,2-dimethyl -1,3-dioxane-4,6-dione was monitored by TLC (PE/EA=1:1, v/v), the total reaction time was 16 h; the reaction mixture was diluted with a large amount of DCM and filtered through a celite pad; the filtrate was washed with saturated ammonium chloride solution once, washed with saturated brine twice, the organic phases were collected and dried over anhydrous sodium sulfate, concentrated by rotary evaporation to obtain compound 3 (dark red liquid), and which was used directly in the next step without further separation and purification.

Synthesis of Compound 5: 36.536 g of 1,6-hexanediol (compound 4) was mixed with compound 3 obtained in the previous step, the mixture was heated to react, and monitored by TLC (PE/EA=1:1, v/v) until compound 3 was consumed completely, and the total reaction time was 18 h; after cooling, a large amount of petroleum ether was added to precipitate 1,6-hexanediol, the mixture was filtered, the filtrate was washed with saturated ammonium chloride solution once and with saturated brine solution three times, and the filtrate was dried over anhydrous sodium sulfate and concentrated by rotary evaporation to obtain a dark red liquid, which was separated and purified by column chromatography (eluted with PE/EA=3:1 (v/v), Rf≈0.5) to obtain 5.417 g of compound 5. An appropriate amount of compound 5 was taken for hydrogen spectrum and mass spectrum detection, and the results were as follows: ¹H NMR (400 MHz, Chloroform-d) δ 4.13 (t, J=6.8 Hz, 2H), 3.64 (t, J=6.4 Hz, 2H), 3.42 (s, 2H), 2.52 (t, J=7.2 Hz, 2H), 1.70-1.62 (m, 2H), 1.62-1.51 (m, 6H), 1.47-1.33 (m, 4H), 1.32-1.19 (m, 16H), 0.87 (t, J=6.8 Hz, 3H). HRMS (ESI, m/z): [M+H]⁺ calcd. For C₂₀H₃₈O₄, 343.28428; found 343.28497.

Synthesis of Compound 6: 5.140 g of compound 5 was mixed with 130 mL of ultradry DCM; 6.255 mL of TEA was added under N₂, followed by addition of 0.187 g of DMAP dissolved in 10 mL of ultradry DCM, and the mixture was stirred for 5 min in an ice bath; 4.523 g of TBSCl (tert-butyldimethylsilyl chloride) was dissolved in 20 mL of ultradry DCM, and added to the mixture under the atmosphere of N₂, after being fully stirred and dissolved, the ice bath was removed, and the mixture was stirred for 75 min at room temperature; consumption of compound 5 (PE/EA=5:1 (v/v)) was monitored by TLC, after consuming completely, the mixture was further extracted with water, the organic phase was collected, then the aqueous phase was extracted with DCM once, the organic phases and DCM phase were combined to obtain a combined solution, the combined solution was washed with saturated brine twice, dried over anhydrous sodium sulfate, concentrated by rotary evaporation, and separated and purified by column chromatography (eluted with PE/EA=20:1 (v/v), Rf≈0.5) to obtain 6.319 g of compound 6. An appropriate amount of compound 6 was taken for mass spectrum detection, and the results were as follows: HRMS (ESI, m/z): [M+H]⁺ calcd. For C₂₆H₅₂O₄Si, 457.37076; found 457.37067.

Synthesis of Compound 7: 6.319 g of compound 6 was mixed with 150 mL of methanol and stirred for 5 min in an ice bath; 0.630 g of sodium borohydride was added, and the mixture was stirred for 2 hours in an ice bath; consumption of compound 6 (PE/EA=20:1, v/v) was monitored by TLC, after consuming completely, the reaction was quenched with saturated sodium bicarbonate solution in an ice bath, then methanol was removed by rotary evaporation, a small amount of pure water was added, the resulting mixture was extracted with ethyl acetate twice, the organic phase was collected, and dried over anhydrous sodium sulfate, concentrated by rotary evaporation, and purified by column chromatography (eluted with PE/EA=10:1 (v/v), Rf ˜0.5) to yield 2.932 g of compound 7. An appropriate amount of compound 7 was taken for hydrogen spectrum and mass spectrum detection, and the results were as follows: ¹H NMR (400 MHz, Chloroform-d) δ 4.08 (t, J=6.8 Hz, 2H), 4.03-3.92 (m, 1H), 3.58 (t, J=6.4 Hz, 2H), 2.99 (d, J=3.6 Hz, 1H), 2.48 (dd, J=16.4, 3.2 Hz, 1H), 2.37 (dd, J=16.4, 8.8 Hz, 1H), 1.67-1.57 (m, 2H), 1.56-1.42 (m, 4H), 1.41-1.19 (m, 22H), 0.91-0.81 (m, 12H). HRMS (ESI, m/z): [M+H]⁺ calcd. For C₂₆H₅₄O₄Si, 459.38641; found 459.38915.

Synthesis of Compound 8: 1.373 g of compound 7 and 0.036 g of DMAP were dissolved in 2 mL of ultradry DCM, and 6.255 mL of TEA was added by syringe, the mixture was stirred for 10 min in an ice bath; hexanoyl chloride dissolved in 2 mL of ultradry DCM was added dropwise under N₂ atmosphere, the reaction was carried out in an ice bath for 10 min, after removing the ice bath, and the reaction was carried out at room-temperature; consumption of compound 7 (PE/EA=10:1, v/v) was monitored by TLC until compound 7 was consumed completely, and the total reaction time was 18 h; the mixture was extracted with saturated ammonium chloride solution, the organic phase was collected and washed with saturated brine solution twice, the organic phases were collected, dried over anhydrous sodium sulfate, concentrated by rotary evaporation, and separated and purified by preparation column chromatography (PE/EA=20:1, v/v, Rf≈0.5) to obtain the compound 8, wherein the yield was 1.100 g, and the yield was 65.8%. An appropriate amount of compound 8 was taken for hydrogen spectrum and mass spectrum detection, and the results were as follows: ¹H NMR (400 MHz, Chloroform-d) δ 5.28-5.15 (m, 1H), 4.09-3.98 (m, 2H), 3.57 (t, J=6.4 Hz, 2H), 2.64-2.30 (m, 3H), 2.24 (t, J=7.6 Hz, 1H), 1.66-1.44 (m, 8H), 1.36-1.17 (m, 28H), 0.91-0.83 (m, 16H). HRMS (ESI, m/z): [M+H]⁺ calcd. For C₃₂H₆₄O₅Si, 557.45958; found 557.45974.

Synthesis of Compound 9: 1.100 g of compound 8 was dissolved in 20 mL of ultradry THF, 1.563 g of tetrabutylammonium fluoride trihydrate was added into the system: consumption of compound 8 (PE/EA=25:1, v/v) was monitored by TLC until compound 8 was consumed completely, and the total reaction time was 7 h; the reaction was quenched with saturated ammonium chloride solution, and the aqueous layer was extracted with DCM for three times, the organic phases were collected, dried over anhydrous sodium sulfate, concentrated by rotary evaporation, and separated and purified by preparation column chromatography (PE/EA=5:1, v/v, Rf˜0.45) to obtain the compound 9, wherein the yield was 0.717 g, and the yield was 81.8%. An appropriate amount of compound 9 was taken for mass spectrum detection, and the results were as follows: HRMS (ESI, m/z): [M+H]⁺ calcd. For C₂₆H₅₀O₅, 443.37310; found 443.37283.

Synthesis of Compound 10: 0.717 g of compound 9 was dissolved in 5 mL of ultradry DCM; then 0.680 g of sodium bicarbonate and 0.867 g of (1,1,1-triacetoxy)-1,1-dihydro-1,2-benziodo-3 (1H)-one were added and the mixture was stirred at room temperature for 0.5 h; consumption of compound 9 (PE/EA=5:1, v/v) was monitored by TLC until compound 9 was consumed completely, the reaction was quenched with saturated sodium thiosulfate solution, and the aqueous layer was extracted with DCM for three times, the organic phases were collected, dried over anhydrous sodium sulfate, concentrated by rotary evaporation, and separated and purified by preparation column chromatography (PE/EA=10:1, v/v, Rf≈0.5) to obtain the compound 10, wherein the yield was 0.263 g, and the yield was 36.9%. An appropriate amount of compound 10 was taken for mass spectrum detection, and the results were as follows: HRMS (ESI, m/z): [M+H]⁺ calcd. For C₂₆H₄₈O₅, 441.35745; found 441.35687.

Synthesis of Compound L0111: 0.336 g of compound 10 was dissolved in 2 mL of ultradry THF; then 0.027 g of 4-amino-1-butanol and 0.163 g of sodium triacetoxyborohydride were added and stirred at room temperature for 12 h; the reaction was quenched with saturated sodium bicarbonate solution, and the aqueous layer was extracted with DCM for three times, the organic phases were collected, dried over anhydrous sodium sulfate, concentrated by rotary evaporation, and separated and purified by preparation column chromatography (DCM/Ultra=3:1, v/v, Rf˜0.5) to obtain compound L0111, wherein the yield was 0.131 g, and the yield was 44.0%. An appropriate amount of compound L0111 was taken for hydrogen spectrum and mass spectrum detection, and the results were as follows: ¹H NMR (400 MHz, Chloroform-d) δ 5.21 (p, J=6.4 Hz, 2H), 4.05 (t, J=6.8 Hz, 4H), 3.57 (s, 2H), 2.67-2.30 (m, 9H), 2.26 (t, J=7.6 Hz, 4H), 1.63-1.55 (m, 20H), 1.36-1.22 (m, 52H), 0.94-0.84 (m, 12H). HRMS (ESI, m/z) [M+H]⁺ calcd for C56H108NO9 938.80186; found 938.80203.

### EXAMPLE 2: PREPARATION OF COMPOUND L0112

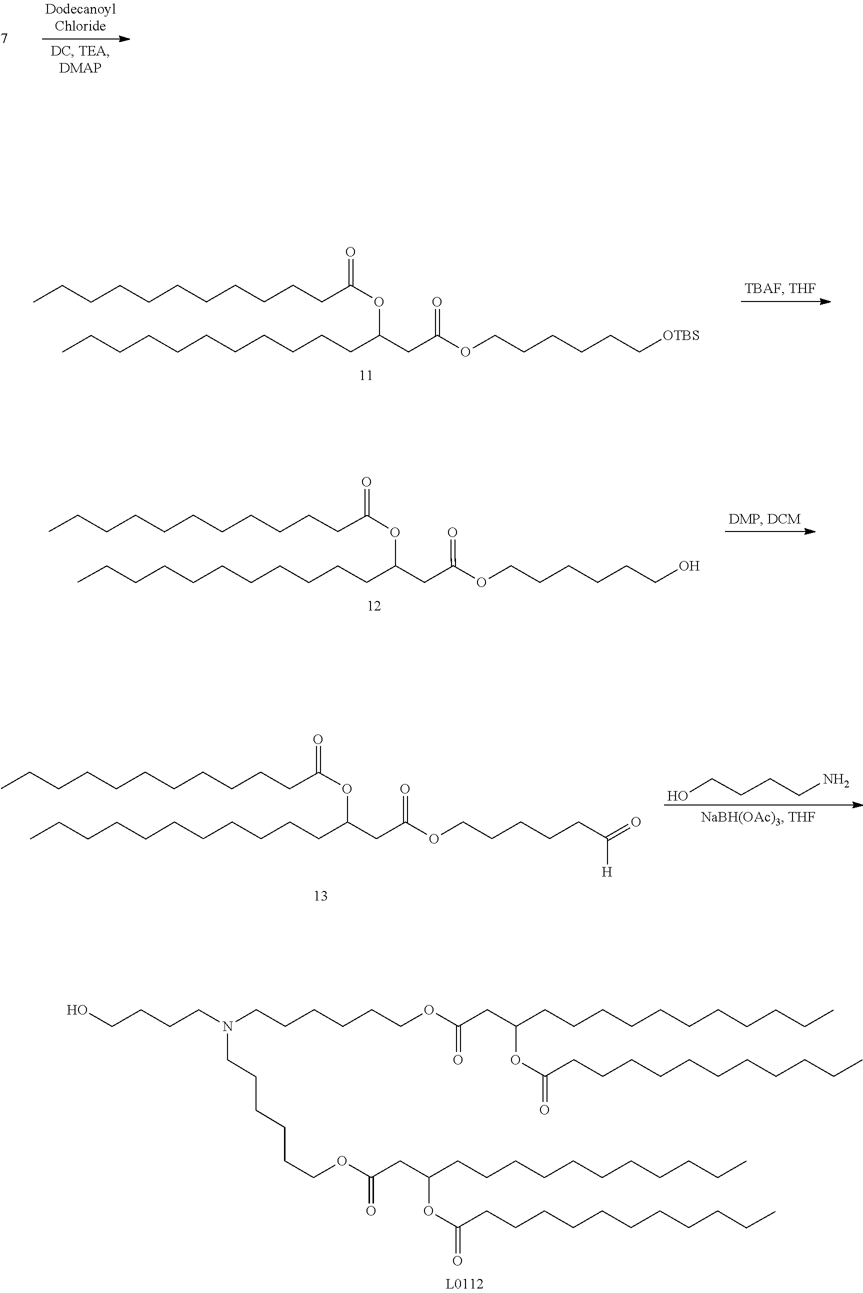 [SMILES: CCCCCCCCCCCC(=O)OC(CCCCCCCCCCC)CC(=O)OCCCCCCO[Si](C)(C)C(C)(C)C ; CCCCCCCCCCCC(=O)OC(CCCCCCCCCCC)CC(=O)OCCCCCCO ; CCCCCCCCCCCC(=O)OC(CCCCCCCCCCC)CC(=O)OCCCCCC=O ; NCCCCO ; CCCCCCCCCCCC(=O)OC(CCCCCCCCCCC)CC(=O)OCCCCCCN(CCCCO)CCCCCCOC(=O)CC(CCCCCCCCCCC)OC(=O)CCCCCCCCCCC]

Synthesis of Compound 11: 0.690 g of compound 7 and 0.010 g of DMAP were dissolved in 2 mL of ultradry DCM, and 3.0 mL of TEA was added by syringe, the mixture was stirred for 10 min in an ice bath; lauroyl chloride dissolved in 2 mL of ultradry DCM was added dropwise under N₂ atmosphere, the reaction was carried out in an ice bath for 10 min, after removing the ice bath, and the reaction was carried out at room-temperature; consumption of compound 7 (PE/EA=10:1, v/v) was monitored by TLC until compound 7 was consumed completely, and the total reaction time was 18 h; the mixture was extracted with saturated ammonium chloride solution, the organic phase was collected and washed with saturated brine solution twice, the organic phases were collected, dried over anhydrous sodium sulfate, concentrated by rotary evaporation, and separated and purified by preparation column chromatography (PE/EA=25:1, v/v, Rf≈0.5) to obtain the compound 11, wherein the yield was 0.544 g, and the yield was 56.5%. An appropriate amount of compound 11 was taken for mass spectrum detection, and the results were as follows: HRMS (ESI, m/z): [M+H]⁺ calcd. For C₃₈H₇₆O₅Si, 641.55348; found 641.55320.

Synthesis of Compound 12: 0.544 g of compound 11 was dissolved in 10 mL of ultradry THF, the solution was mixed with a solution of 0.318 g of tetrabutylammonium fluoride trihydrate in ultradry THF with a concentration of 1M, the mixture was reacted at room temperature; consumption of compound 8 (PE/EA=25:1, v/v) was monitored by TLC until compound 8 was consumed completely, and the total reaction time was 18 h; the reaction was quenched with saturated ammonium chloride solution, and the aqueous layer was extracted with DCM for three times, the organic phases were collected, dried over anhydrous sodium sulfate, concentrated by rotary evaporation, and separated and purified by preparation column chromatography (PE/EA=5:1, v/v, Rf˜0.5) to obtain the compound 12, wherein the yield was 0.384 g, and the yield was 85.7%. An appropriate amount of compound 12 was taken for mass spectrum detection, and the results were as follows: HRMS (ESI, m/z): [M+H]⁺ calcd. For C₃₂H₆₂O₅, 527.46700; found 527.46789.

Synthesis of Compound 13: 0.384 g of compound 12 was dissolved in 5 mL of ultradry DCM; then 0.307 g of sodium bicarbonate and 0.383 g of (1,1,1-triacetoxy)-1,1-dihydro-1,2-benziodo-3 (1H)-one were added and stirred at room temperature for 0.5 h; consumption of compound 9 (PE/EA=5:1, v/v) was monitored by TLC until compound 9 was consumed completely, the reaction was quenched with saturated sodium thiosulfate solution, and the aqueous layer was extracted with DCM for three times, the organic phases were collected, dried over anhydrous sodium sulfate, concentrated by rotary evaporation, and separated and purified by preparation column chromatography (PE/EA=8:1, v/v, Rf≈0.5) to obtain the compound 13, wherein the yield was 0.108 g, and the yield was 27.3%.

Synthesis of Compound L0112: 0.108 g of compound 13 was dissolved in 2 mL of ultradry THF; then 0.008 g of 4-amino-1-butanol and 0.044 g of sodium triacetoxyborohydride were added and stirred at room temperature for 12 h; the reaction was monitored by TLC (DCM/Ultra=3:1, v/v) (Ultra=DCM:methanol:ammonia=75:22:3), a spot with a high concentration at Rf˜0.5 was found, the reaction was quenched with saturated sodium bicarbonate solution, and the aqueous layer was extracted with DCM for three times, the organic phases were collected, dried over anhydrous sodium sulfate, concentrated by rotary evaporation, and separated and purified by preparation column chromatography (DCM/Ultra=3:1, v/v, Rf˜0.5) to obtain compound L0112, wherein the yield was 0.022 g, and the yield was 23.2%. An appropriate amount of compound L0112 was taken for hydrogen spectrum and mass spectrum detection, and the results were as follows: ¹H NMR (400 MHz, Chloroform-d) δ 5.20 (p, J=6.2 Hz, 2H), 4.04 (t, J=6.8 Hz, 4H), 3.54 (t, J=4.4 Hz, 2H), 2.60-2.47 (m, 4H), 2.47-2.35 (m, 6H), 2.25 (t, J=7.6 Hz, 4H), 1.61 (dt, J=14.4, 7.2 Hz, 16H), 1.51-1.41 (m, 4H), 1.38-1.20(m, 76H), 0.87 (t, J=6.8 Hz, 12H); HRMS (ESI, m/z) [M+H]⁺ calcd for C68H132NO9: 1106.98966; found: 1106.99201.

### EXAMPLE 3: PREPARATION OF COMPOUND L0113

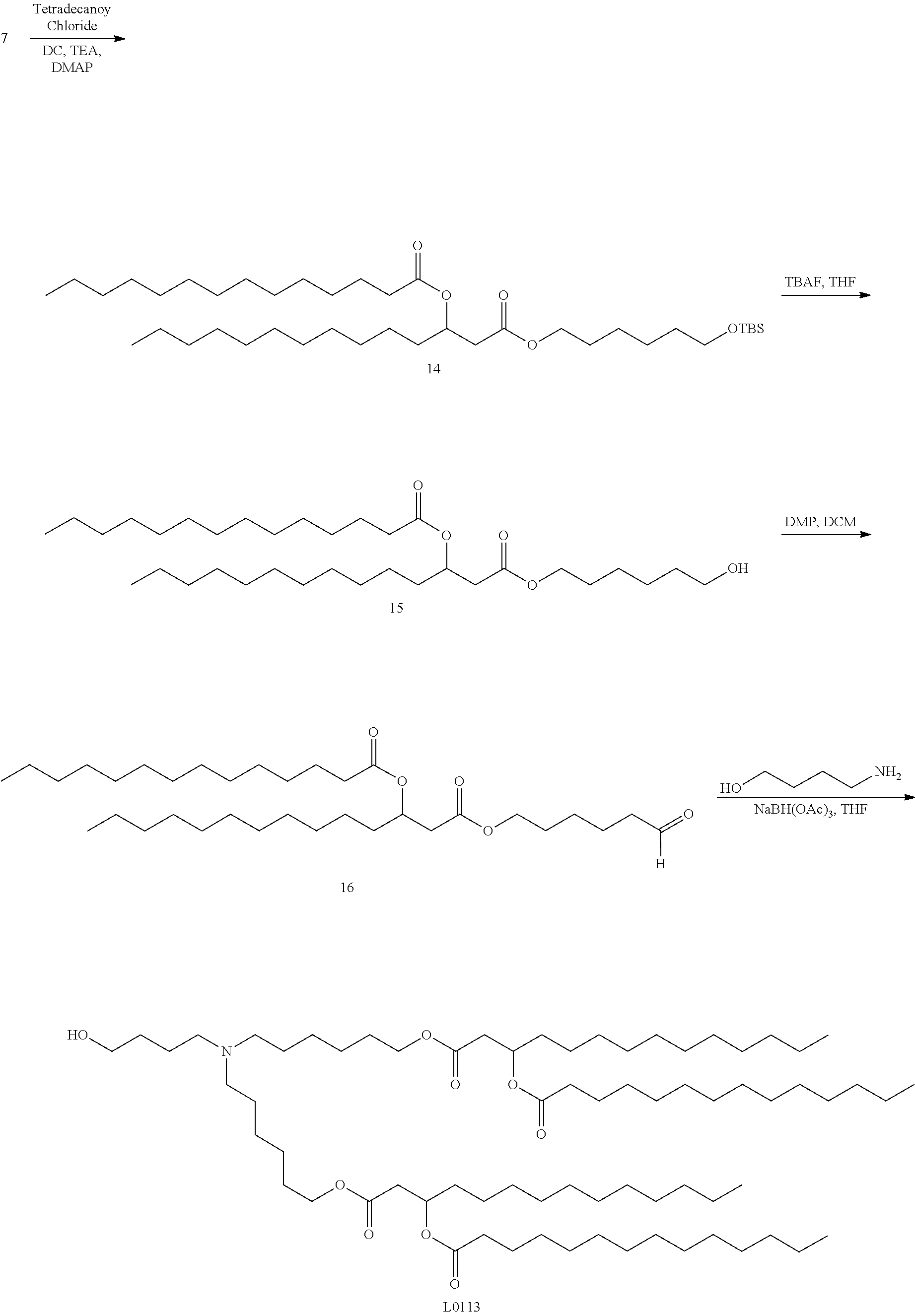 [SMILES: CCCCCCCCCCCCCC(=O)OC(CCCCCCCCCCC)CC(=O)OCCCCCCO[Si](C)(C)C(C)(C)C ; CCCCCCCCCCCCCC(=O)OC(CCCCCCCCCCC)CC(=O)OCCCCCCO ; CCCCCCCCCCCCCC(=O)OC(CCCCCCCCCCC)CC(=O)OCCCCCC=O ; NCCCCO ; CCCCCCCCCCCCCC(=O)OC(CCCCCCCCCCC)CC(=O)OCCCCCCN(CCCCO)CCCCCCOC(=O)CC(CCCCCCCCCCC)OC(=O)CCCCCCCCCCCCC]

Synthesis of Compound 14: 1.377 g of compound 7 and 0.039 g of DMAP were mixed with 1.5 mL of TEA dissolved in 2 mL of ultradry DCM, the mixture was stirred for 10 min in an ice bath; 800 mg of myristoyl chloride dissolved in 2 mL of ultradry DCM was added dropwise under N₂ atmosphere, the reaction was carried out in an ice bath for 10 min, after removing the ice bath, and the reaction was carried out at room temperature; consumption of compound 7 (PE/EA=10:1, v/v) was monitored by TLC until compound 7 was consumed completely, and the total reaction time was 18 h; the mixture was extracted with saturated ammonium chloride solution, the organic phase was collected and washed with saturated brine solution twice, the organic phases were collected, dried over anhydrous sodium sulfate, concentrated by rotary evaporation, and separated and purified by preparation column chromatography (PE/EA=50:1, v/v, Rf˜0.5) to obtain the compound 14, wherein the yield was 1.572 g, and the yield was 78.3%. HRMS (ESI, m/z): [M+H]⁺ calcd. For C₄₀H₈₀O₅Si, 669.58478; found 669.58466.

Synthesis of Compound 15: 141.572 g of compound 14 was placed in a three-neck flask equipped with a two-way valve on the straight port, the two diagonal ports was blocked with rubber stoppers, and 10 mL of ultradry THF was added and stirred to dissolve the mixture; 1.853 g of tetrabutylammonium fluoride trihydrate was formulated to a 1M solution in ultradry THF, and the reaction was carried out at room temperature; consumption of compound 8 (PE/EA=25:1, v/v) was monitored by TLC until compound 8 was consumed completely, and the total reaction time was 18 h; the reaction was quenched with saturated ammonium chloride solution, and the aqueous layer was extracted with DCM for three times, the organic phases were collected, dried over anhydrous sodium sulfate, concentrated by rotary evaporation, and separated and purified by preparation column chromatography (PE/EA=5:1, v/v, Rf˜0.5) to obtain the compound 15, wherein the yield was 0.902 g, and the yield was 69.2%. HRMS (ESI, m/z): [M+H]⁺ calcd. For C₃₄H₆₆O₅, 555.49830; found 555.49822.

Synthesis of Compound 16: 0.902 g of compound 15 was dissolved in 5 mL of ultradry DCM; then 0.686 g of sodium bicarbonate and 0.861 g of (1,1, 1-triacetoxy)-1,1-dihydro-1,2-benziodo-3 (1H)-one were added and stirred at room temperature for 0.5h; consumption of compound 9 (PE/EA=5:1, v/v) was monitored by TLC until compound 9 was consumed completely, the reaction was quenched with saturated sodium thiosulfate solution, and the aqueous layer was extracted with DCM for three times, the organic phases were collected, dried over anhydrous sodium sulfate, concentrated by rotary evaporation, and separated and purified by preparation column chromatography (PE/EA=8:1, v/v, Rf˜0.5) to obtain the compound 16, wherein the yield was 0.622 g, and the yield was 69.0%. HRMS (ESI, m/z): [M+H]⁺ calcd. For C₃₄H₆₆O₅, 553.48265; found 553.48342.

Synthesis of Compound L0113: 0.133 g of compound 16 was dissolved in 2 mL of ultradry THF; then 0.009 g of 4-amino-1-butanol and 0.054 g of sodium triacetoxyborohydride were added and stirred at room temperature for 12 h; the reaction was monitored by TLC (DCM/Ultra=3:1, v/v) (Ultra=DCM:methanol:ammonia=75:22:3), a spot with a high concentration at Rf˜0.5 was found, the reaction was quenched with saturated sodium bicarbonate solution, and the aqueous layer was extracted with DCM for three times, the organic phases were collected, dried over anhydrous sodium sulfate, concentrated by rotary evaporation, and separated and purified by preparation column chromatography (DCM/Ultra=3:1, v/v, Rf≈0.5) to obtain the compound L0113, wherein the yield was 0.062 g, and the yield was 53.4%. An appropriate amount of compound L0113 was taken for hydrogen spectrum and mass spectrum detection, and the results were as follows: ¹H NMR (400 MHz, Chloroform-d) δ 5.21 (p, J=6.4 Hz, 2H), 4.05 (t, J=6.8 Hz, 4H), 3.58 (s, 2H), 2.75-2.32 (m, 8H), 2.26 (t, J=7.6 Hz, 4H), 1.66-1.54 (m,26H), 1.43-1.18 (m, 80H), 0.88 (t, J=6.8 Hz, 12H). HRMS (ESI, m/z): [M+H]⁺ calcd. For C₇₂H₁₄₀NO₉, 1163.05226; found 1163.05738.

### EXAMPLE 4: PREPARATION Of COMPOUND L0114

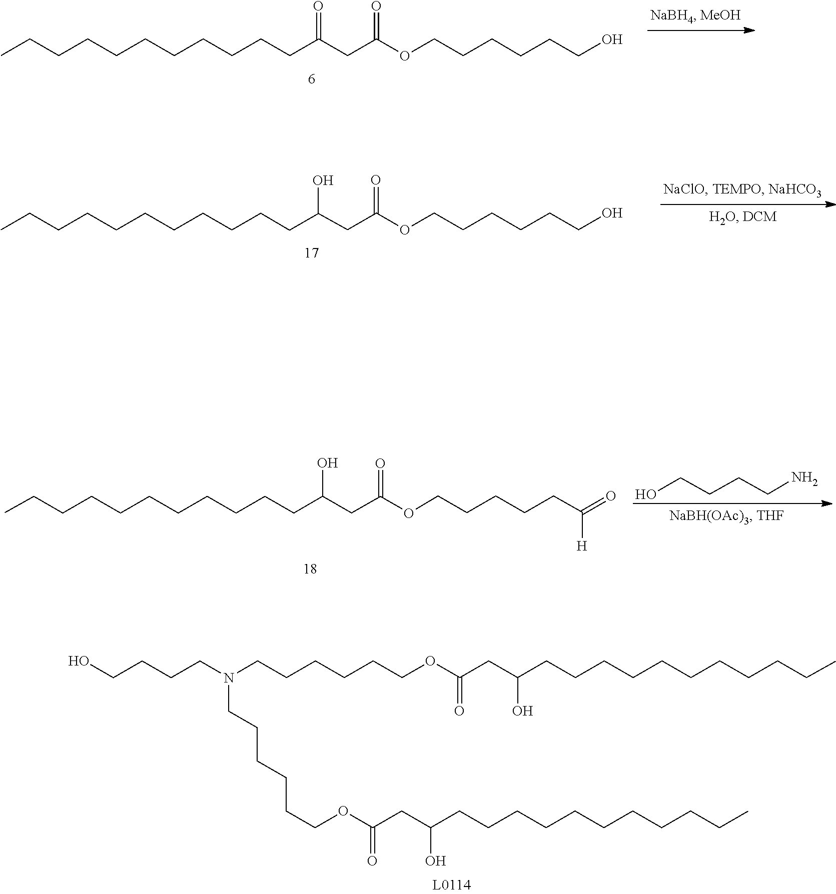

Synthesis of Compound 17: 1.045 g of compound 5 was dissolved in 10 mL of methanol and then 40 ml of methanol was supplemented, the mixture was stirred for 5 min in an ice bath; 0.137 g of sodium borohydride was added, and the mixture was stirred for 2 hours in an ice bath; consumption of compound 5 (PE/EA=5:1, v/v) was monitored by TLC, after consuming completely, the reaction was quenched with saturated sodium bicarbonate solution in an ice bath, methanol was removed by rotary evaporation, an appropriate amount of pure water was added, the resulting mixture was extracted with ethyl acetate twice, the organic phase was collected, and dried over anhydrous sodium sulfate, concentrated by rotary evaporation, and purified by column chromatography (PE/EA=2:1 (v/v), Rf≈0.35) to obtain compound 17, wherein the yield was 0.950 g, and the yield was 91.9%. HRMS (ESI, m/z): [M+H]⁺ calcd. For C₂₀H₄₀O₄, 345.29993; found 345.29975.

Synthesis of Compound 18: 0.950 g of compound 17 was dissolved in 30 mL of ultradry DCM, 15 mL of saturated sodium bicarbonate solution was added, followed by addition of 0.122 g of potassium bromide and 0.045 g of TEMPO, the mixture was stirred in an ice bath for 10 min; after adding 3 mL of sodium hypochlorite aqueous solution (the available chlorine ≥5%), the mixture was monitored by TLC (PE/EA=5:1, v/v) every 2 min, the total reaction time was 10 min; the reaction was quenched with saturated sodium thiosulfate solution, the aqueous layer was extracted with DCM, the organic phase was collected, and dried over anhydrous sodium sulfate, concentrated by rotary evaporation, and purified by column chromatography (PE/EA=4:1 (v/v), Rf≈0.5) to obtain compound 18, wherein the yield was 0.086 g, and the yield was 9.1%. HRMS (ESI, m/z): [M+H]⁺ calcd. For C₂₀H₃₈O₄, 343.28428; found 343.28415.

Synthesis of Compound L0114: 0.086 g of compound 18 was dissolved in 2 mL of ultradry THF; then 0.009 g of 4-amino-1-butanol and 0.056 g of sodium triacetoxyborohydride were added and stirred at room temperature for 12 h; the reaction was monitored by TLC (DCM/Ultra=3:1, v/v) (Ultra=DCM:methanol:ammonia=75:22:3), a spot with a high concentration at Rf˜0.5 was found, the reaction was quenched with saturated sodium bicarbonate solution, and the aqueous layer was extracted with DCM for three times, the organic phases were collected, dried over anhydrous sodium sulfate, concentrated by rotary evaporation, and separated and purified by preparation column chromatography (DCM/Ultra=3:1, v/v, Rf˜0.5) to obtain compound L0114, wherein the yield was 0.043 g, and the yield was 55.1%. An appropriate amount of compound L0114 was taken for hydrogen spectrum and mass spectrum detection, and the results were as follows: ¹H NMR (400 MHz, Chloroform-d) δ 4.07 (tt, J=11.0, 5.6 Hz, 4H), 3.98 (tt, J=8.0, 4.0 Hz, 2H), 3.56 (t, J=5.2 Hz, 2H), 2.75-2.49 (m, 6H), 2.50-2.42 (m, 2H), 2.37 (dd, J=16.0, 9.2 Hz, 2H), 1.77-1.43 (m, 14H), 1.43-1.16 (m, 46H), 0.85 (t, J=6.8 Hz, 6H). HRMS (ESI, m/z): [M+H]⁺ calcd. For C₄₄H₈₇NO₇, 742.65553; found 742.65491.

### EXAMPLE 5: PREPARATION OF COMPOUND L0115

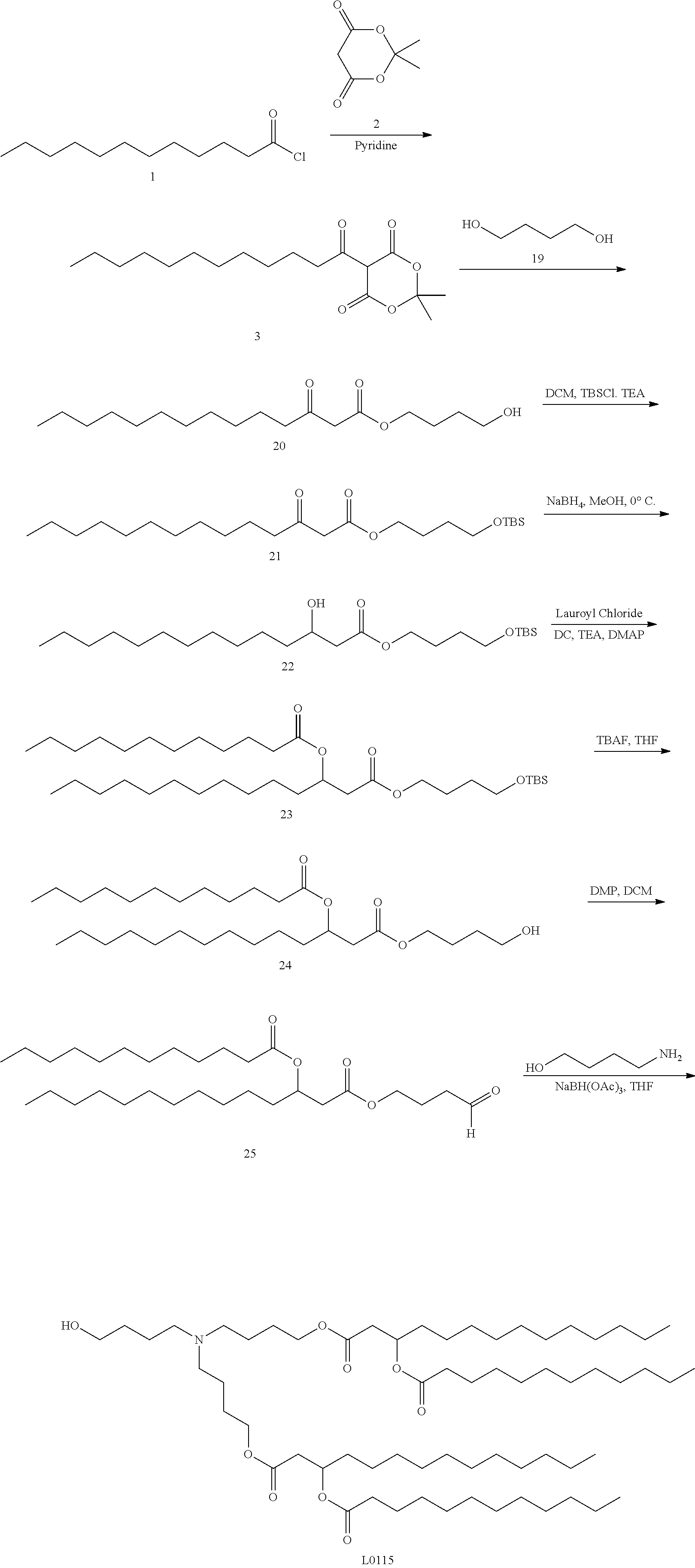

14.070 g of 2,2-dimethyl-1,3-dioxane-4,6-dione (compound 2) and 24.826 mL of lauroyl chloride (compound 1) were dissolved in 300 mL of ultradry DCM, and the mixture was stirred for 5 min in an ice bath; then 15.706 mL of pyridine was added, the mixture was stirred for 1 h in an ice bath; after removing the ice bath, the reaction was continued, and consumption of 2,2-dimethyl-1,3-dioxane-4,6-dione was monitored by TLC (PE/EA=1:1, v/v), the total reaction time was 6 h; DCM was removed by rotary evaporation and an appropriate amount of ethyl acetate was added, the mixture was filtered through a celite pad; the filtrate was washed with 100 mL of saturated ammonium chloride solution once, washed with saturated brine twice (100 mL×2), the organic phases were collected and dried over anhydrous sodium sulfate, concentrated by rotary evaporation to obtain compound 3 (dark red liquid), and which was used directly in the next step without further separation and purification.

27.552 g of 1,4-butanediol (compound 19) was mixed with compound 3 obtained in the previous step, and the mixture was heated to react, the reaction temperature was 80-100° C., consumption of compound 3 was monitored by TLC (PE/EA=1:1, v/v), after consuming completely, and the total reaction time is 11.5 h; after cooling, 1500 mL of DCM was added, the mixture was washed with 300 mL of saturated ammonium chloride solution once and with saturated brine twice (500 mL×2), dried over anhydrous sodium sulfate and concentrated by rotary evaporation to give a dark red liquid, which was purified by column chromatography (PE/EA=3:1, v/v, Rf≈0.5) to give compound 20, wherein the yield was 7.247 g, and the yield was 45.29%. HRMS (ESI, m/z): [M+Na]⁺ calcd. For C₁₈H₃₄O₄, 337.23493; found 337.23532.

7.247 g of compound 20 was dissolved in 200 mL of ultradry DCM; 9.611 mL of TEA was added under N₂, followed by addition of 0.282 g of DMAP dissolved in 300 mL of ultradry DCM, and the mixture was stirred for 5 min in an ice bath; 7.002 g of TBSCl was dissolved in 300 mL of ultradry DCM, and added to the mixture under the atmosphere of N₂, after being fully stirred and dissolved, the ice bath was removed, and the mixture was stirred for 75 min at room temperature; consumption of compound 20 (PE/EA=5:1 (v/v)) was monitored by TLC, after consuming completely, the mixture was washed with water in a separatory funnel, the organic phase was collected, then the aqueous phase was extracted with DCM once, the organic phases were combined, the combined solution was washed with saturated brine twice (100 mL×2), dried over anhydrous sodium sulfate, concentrated by rotary evaporation, and separated and purified by column chromatography (eluted with PE/EA=20:1 (v/v), Rf˜0.5) to obtain compound 21, wherein the yield was 9.234 g, and the yield was 93.44%. HRMS (ESI, m/z): [M+H]⁺ calcd. For C₂₄H₄₈O₄Si, 429.33946; found 429.33914.

2.240 g of compound 21 was dissolved in 10 mL of methanol, the mixture was stirred for 5 min in an ice bath; 0.237 g of sodium borohydride was added, and the mixture was stirred for 2 hours in an ice bath; consumption of compound 21 (PE/EA=5:1, v/v) was monitored by TLC, after consuming completely, the reaction was quenched with saturated sodium bicarbonate solution in an ice bath, methanol was removed by rotary evaporation, 150 mL of pure water was added, the resulting mixture was extracted with ethyl acetate twice (50 mL×2), the organic phase was collected, and dried over anhydrous sodium sulfate, concentrated by rotary evaporation, and purified by column chromatography (PE/EA=10:1 (v/v), Rf˜0.5) to obtain compound 22, wherein the yield was 0.957 g, and the yield was 42.56%. HRMS (ESI, m/z): [M+H]⁺ calcd. For C₂₄H₅₀O₄Si, 431.35511; found 431.35559.

0.957 g of compound 22 and 0.027 g of DMAP were mixed with 1.234 mL of TEA dissolved in 50 mL of ultradry DCM, the mixture was stirred for 10 min in an ice bath; 0.770 mL of lauroyl chloride dissolved in 20 mL of ultradry DCM was added under N₂ atmosphere, the reaction was carried out in an ice bath for 10 min, after removing the ice bath, and the reaction was carried out at room temperature; consumption of compound 22 (PE/EA=10:1, v/v) was monitored by TLC until compound 22 was consumed completely, and the total reaction time was 18 h; the mixture was extracted with 30 mL of saturated ammonium chloride solution, the organic phase was collected and washed with saturated brine solution twice (20 mL×2), the organic phases were collected, dried over anhydrous sodium sulfate, concentrated by rotary evaporation, and separated and purified by preparation column chromatography (PE/EA=25:1, v/v, Rf˜0.5) to obtain compound 23, wherein the yield was 1.095 g, and the yield was 80.5%. HRMS (ESI, m/z): [M+H]⁺ calcd. For C₃₆H₇₂O₅Si, 613.52218; found 613.52265.

1.095 g of compound 23 was dissolved in 30 mL of ultradry THF, the solution was mixed with a solution of 1.415 g of tetrabutylammonium fluoride trihydrate in ultradry THF with a concentration of IM, the mixture was reacted at room temperature; consumption of compound 23 (PE/EA=25:1, v/v) was monitored by TLC until compound 23 was consumed completely, and the total reaction time was 6 h; the reaction was quenched with saturated ammonium chloride solution, the organic phase was collected, washed with saturated brine solution twice (20 mL×2), dried over anhydrous sodium sulfate, concentrated by rotary evaporation, and separated and purified by preparation column chromatography (PE/EA=5:1, v/v, Rf≈0.5) to obtain compound 24, wherein the yield was 0.417 g, and the yield was 46.7%. HRMS (ESI, m/z): [M+H]⁺ calcd. For C₃₀H₅₈O₅, 499.43570; found 499.43604.

0.417 g of compound 24 was dissolved in 10 mL of ultradry DCM; then 0.356 g of sodium bicarbonate and 0.472 g of (1,1,1-triacetoxy)-1,1-dihydro-1,2-benziodo-3 (1H)-one were added and stirred at room temperature for 0.5 h; consumption of compound 24 (PE/EA=5:1, v/v) was monitored by TLC until compound 24 was consumed completely, the reaction was quenched with saturated sodium thiosulfate solution, and the aqueous layer was extracted with DCM for three times, the organic phases were collected, dried over anhydrous sodium sulfate, concentrated by rotary evaporation, and separated and purified by preparation column chromatography (PE/EA=10:1, v/v, Rf≈0.5) to obtain the compound 25, wherein the yield was 0.233 g, and the yield was 56.1%. ¹H NMR (400 MHz, Chloroform-d) δ9.78 (s, 1H), 5.26-5.15 (m, 1H), 4.09 (t, J=6.4 Hz, 2H), 2.55 (q, J=6.8 Hz, 4H), 2.26 (t, J=7.6 Hz, 2H), 2.03-1.90 (m, 2H), 1.69-1.51 (m, 4H), 1.36-1.15 (m, 34H), 0.87 (t, J=6.8 Hz, 6H). HRMS (ESI, m/z): [M+H]⁺ calcd. For C₃₀H₅₆O₅, 497.42005; found 497.41992.

0.108 g of compound 25 was dissolved in 10 mL of ultradry THF; then 0.010 g of 4-amino-1-butanol and 0.052 g of sodium triacetoxyborohydride were added and stirred at room temperature for 12 h; the reaction was monitored by TLC (DCM/Ultra=3:1. v/v) (Ultra=DCM:methanol:ammonia=75:22:3), two adjacent spots with high concentrations at Rf≈0.5 were found, the reaction was quenched with saturated sodium bicarbonate solution, and the organic phases were collected, the aqueous layer was extracted with DCM for three times, the organic phases were collected, dried over anhydrous sodium sulfate, concentrated by rotary evaporation, and separated and purified by preparation column chromatography (DCM/Ultra=3:1, v/v, Rf˜0.5) to obtain the compound L0115, wherein the yield was 0.091 g, and the yield was 86.7%. ¹H NMR (400 MHz, Chloroform-d) δ 5.26-5.13 (m, 2H), 4.06 (t, J=6.4 Hz, 4H), 3.60-3.53 (m, 2H), 2.70-2.37 (m, 10H), 2.26 (t, J=7.6 Hz, 4H), 1.78-1.43 (m, 20H), 1.36-1.17 (m, 68H), 0.87 (t, J=6.8 Hz, 12H). HRMS (ESI, m/z): [M+H]⁺ calcd. For C₆₄H₁₂₃NO₉, 1050.92706; found 1050.92618.

### EXAMPLE 6: PREPARATION Of COMPOUND L0116, COMPOUND L0117, COMPOUND L0118, COMPOUND L0119, COMPOUND L0120, COMPOUND L0121, COMPOUND L0122, COMPOUND L0123, COMPOUND L0124, COMPOUND L0125, COMPOUND L0126, COMPOUND L0127, COMPOUND L0128, COMPOUND L0129 and COMPOUND L0130-L0140

compound L0116, compound L0117, compound L0118, compound L0119, compound L0120, compound L0121, compound L0122, compound L0123, compound L0124, compound L0125, compound L0126, compound L0127, compound L0128, compound L0129, compound L0130, compound L0131, compound L0132, compound L0133, compound L0134, compound L0135, compound L0136 and compound L0137 provided herein are all obtained by reductive amination of commercially available amines and corresponding aldehyde compounds, the methods may refer to the preparation method of compounds L0111-L0115.

According to the structural characteristics of the target product, 0.1 mmol of commercially available amine (shown as compound Al to compound All, respectively) and 0.13×n mmol of aldehyde (shown as compound 10, compound 13, compound 16, compound 18, or compound 25, respectively, where n is the number of active hydrogen of the amine) were dissolved in 2 mL of tetrahydrofuran, 0.13×n mmol of sodium triacetoxyborohydride was added to the above solution and stirred at room temperature for 12 h. And 5 mL of saturated sodium bicarbonate solution was added to quench the reaction, extracted with 10 mL of DCM twice, the organic phases were combined, and dried over anhydrous sodium sulfate, concentrated by rotary evaporation, and the residue was purified by silica gel column chromatography to obtain the target products L0116-L0137.

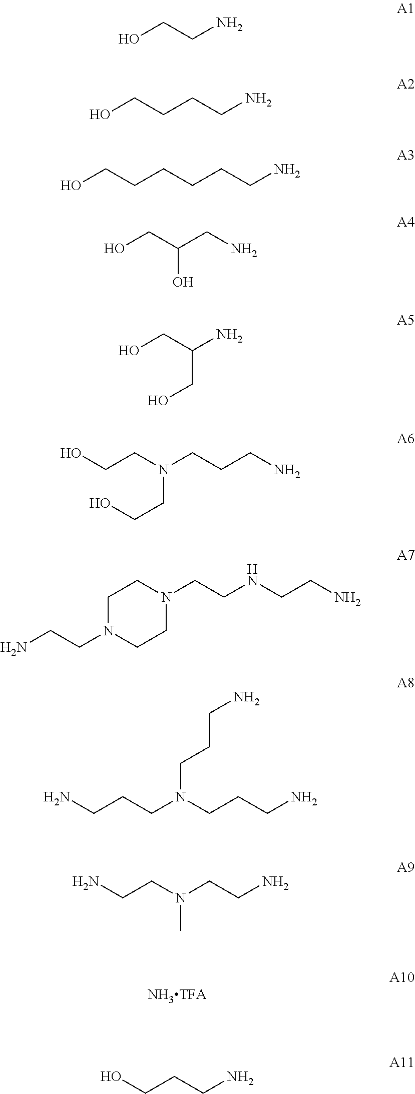

An appropriate amount of compound L0116, Compound L0117, Compound L0118, Compound L0119, Compound L0120, Compound L0121, Compound L0122, Compound L0123, Compound L0124, Compound L0125, Compound L0126, Compound L0127, Compound L0128, Compound L0129, Compound L0130, Compound L0131, Compound L0132, Compound L0133, Compound L0134, Compound L0135, Compound L0136 and Compound L0137 were taken for hydrogen spectrum and mass spectrum detection, and the results were as follows:

Compound L0116: ¹H NMR (400 MHz, Chloroform-d) δ 5.23-5.06 (m, 2H), 3.98 (t, J=6.8 Hz, 4H), 3.82-3.72 (m, 1H), 3.67 (dd, J=11.6, 4.0 Hz, 1H), 3.63-3.52 (m, 1H), 3.45 (dd, J=11.6, 4.4 Hz, 1H), 2.83-2.31 (m, 10H), 2.20 (t, J=7.6 Hz, 4H), 1.58-1.46 (m, 12H), 1.44-1.08 (m, 56H), 0.87-0.77 (m, 12H). HRMS (ESI, m/z): [M+H]⁺ calcd. For C₅₅H₁₀₅NO₁₀, 940.78112; found 940.78070.

Compound L0117: ¹H NMR (400 MHz, Chloroform-d) δ 5.29-5.15 (m, 2H), 4.05 (t, J=6.8 Hz, 4H), 3.73-3.57 (m, 2H), 2.80-2.68 (m, 2H), 2.66-2.46 (m, 8H), 2.26 (t, J=7.6 Hz, 4H), 1.67-1.51 (m, 14H), 1.51-1.10 (m, 54H), 0.93-0.84 (m, 12H). HRMS (ESI, m/z): [M+H]⁺ calcd. For C₅₄H₁₀₃NO₉, 910.77056; found 910.77051.

Compound L0118: ¹H NMR (400 MHz, Chloroform-d) δ 5.26-5.15 (m, 2H), 4.04 (t, J=6.8 Hz, 4H), 2.63-2.36 (m, 15H), 2.36-2.32 (m, 3H), 2.29-1.93 (m, 10H), 1.73-1.50 (m, 14H), 1.49-1.39 (m, 4H), 1.39-1.15 (m, 52H), 0.93-0.82 (m, 12H). HRMS (ESI, m/z): [M+H]⁺ calcd. For C₆₀H₁₁₆N₃O₈, 1006.87569; found 1006.87518.

Compound L0119: ¹H NMR (400 MHz, Chloroform-d) δ 5.28-5.14 (m, 2H), 4.04 (t, J=6.8 Hz, 4H), 2.67-2.32 (m, 17H), 2.32-1.93 (m, 10H), 1.77-1.49 (m, 12H), 1.49-1.39 (m, 4H), 1.39-1.14 (m, 52H), 0.93-0.82 (m, 12H). HRMS (ESI, m/z): [M+H]⁺ calcd. For C₅₉H₁₁₄N₃O₈, 992.86004; found 992.86032.

Compound L0120: ¹H NMR (400 MHz, Chloroform-d) δ 5.29-5.16 (m, 2H), 4.07 (t, J=6.4 Hz, 4H), 3.85-3.67 (m, 2H), 3.50 (dd, J=11.2, 4.0 Hz, 1H), 2.82-2.36 (m, 10H), 2.26 (t, J=7.6 Hz, 4H), 1.70-1.46 (m, 16H), 1.35-1.23 (m, 68H), 0.88 (t, J=6.8 Hz, 12H). HRMS (ESI, m/z): [M+H]⁺ calcd. For C₆₃H₁₂₁NO₁₀, 1052.90632; found 1052.90719.

Compound L0121: ¹H NMR (400 MHz, Chloroform-d) δ 5.27-5.15 (m, 2H), 4.07 (t, J=6.4 Hz, 4H), 3.57 (t, J=5.2 Hz, 2H), 2.72-2.40 (m, 9H), 2.26 (t, J=7.6 Hz, 4H), 1.70-1.43 (m, 16H), 1.37-1.18 (m, 68H), 0.87 (t, J=6.8 Hz, 12H). HRMS (ESI, m/z): [M+H]⁺ calcd. For C₆₂H₁₁₉NO₉, 1022.89576; found 1022.89627.

Compound L0122: ¹H NMR (400 MHz, Chloroform-d) δ 5.26-5.12 (m, 2H), 4.13-4.00 (m, 4H), 3.63 (t, J=5.2 Hz, 4H), 2.73-2.43 (m, 16H), 2.25 (t, J=7.6 Hz, 4H), 1.78-1.46 (m, 18H), 1.41-1.13 (m, 68H), 0.86 (t, J=6.8 Hz, 12H). HRMS (ESI, m/z): [M+H]⁺ calcd. For C₆₇H₁₃₀N₂O₁₀, 1123.98449; found 1123.98449.

Compound L0123: ¹H NMR (400 MHz, Chloroform-d) δ 5.28-5.15 (m, 2H), 4.06 (t, J=6.4 Hz, 4H), 3.63 (t, J=6.4 Hz, 2H), 2.70-2.31 (m, 10H), 2.25 (t, J=7.6 Hz, 4H), 1.69-1.43 (m, 20H), 1.42-1.17 (m, 72H), 0.87 (t, J=6.8 Hz, 12H). HRMS (ESI, m/z): [M+H]⁺ calcd. For C₆₆H₁₂₇NO₉, 1078.95836; found 1078.96087.

Compound L0124: ¹H NMR (400 MHz, Chloroform-d) δ 5.28-5.16 (m, 2H), 4.05 (t, J=6.8 Hz, 4H), 3.63 (d, J=6.8 Hz, 4H), 3.03 (d, J=8.0 Hz, 1H), 2.57 (pd, J=15.2, 6.8 Hz, 8H), 2.26 (t, J=7.6 Hz, 4H), 1.61 (pd, J=7.2, 4.3 Hz, 12H), 1.43-1.15 (m, 56H), 0.93-0.83 (m, 12H). HRMS (ESI, m/z): [M+H]⁺ calcd. For C₅₅H₁₀₅NO₁₀, 940.78112; found 940.78279.

Compound L0125: ¹H NMR (400 MHz, Chloroform-d) δ 5.27-5.14 (m, 2H), 4.03 (t, J=6.8 Hz, 4H), 3.62 (t, J=6.4 Hz, 2H), 2.91-2.66 (m, 6H), 2.59-2.48 (m, 4H), 2.25 (t, J=7.6 Hz, 4H), 1.77-1.48 (m, 20H), 1.45-1.19 (m, 56H), 0.87 (q, J=6.8 Hz, 12H). HRMS (ESI, m/z): [M+H]⁺ calcd. For C₅₈H₁₁₁NO₉, 966.83316; found 966.83516.

Compound L0126: ¹H NMR (400 MHz, Chloroform-d) δ 5.19 (p, J=6.4 Hz, 5H), 4.12-3.98 (m, 10H), 2.78-2.44 (m, 40H), 2.32-2.17 (m, 10H), 1.77-1.49 (m, 30H), 1.48-1.16 (m, 220H), 0.86 (t, J=6.8 Hz, 30H). HRMS (ESI, m/z): [M+2H]⁺ calcd. For C₁₈₀H₃₄₅N₅O₂₀, m/z: 1450.31560, found 1449.8233.

Compound L0127: ¹H NMR (400 MHz, Chloroform-d) δ 5.21 (p, J=6.4 Hz, 5H), 4.04 (t, J=6.8 Hz, 10H), 2.73-2.32 (m, 40H), 2.26 (t, J=7.6 Hz, 10H), 1.68-1.49 (m, 30H), 1.39-1.19 (m, 140H), 0.88 (q, J=6.4 Hz, 30H). HRMS (ESI, m/z): [M+3H]⁺ calcd. For C₂₁₀H₄₀₂N₄O₂₄, 780.33749, found: 780.34277.

Compound L0128: ¹H NMR (400 MHz, Chloroform-d) δ 5.20 (p, J=6.4 Hz, 5H), 4.12-4.01 (m, 10H), 2.70-2.42 (m, 36H), 2.26 (t, J=7.6 Hz, 10H), 2.21-2.17 (m, 4H), 1.67-1.50 (m, 30H), 1.51-1.15 (m, 200H), 0.87 (t, J=6.8 Hz, 30H).

Compound L0129: ¹H NMR (400 MHz, Chloroform-d) δ 5.27-5.14 (m, 3H), 4.03 (t, J=6.8 Hz, 6H), 2.75-2.48 (m, 10H), 2.25 (t, J=7.6 Hz, 6H), 1.78-1.44 (m, 24H), 1.41-1.18 (m, 78H), 0.87 (q, J=6.8 Hz, 18H). HRMS (ESI, m/z): [M+H]⁺ calcd. For C₇₈H₁₄₇NO₁₂, 1291.09960; found 1291.10025.

Compound L0130: ¹H NMR (400 MHz, Chloroform-d) δ 5.20 (p, J=6.4 Hz, 3H), 4.05 (t, J=6.8 Hz, 6H), 2.63-2.48 (m, 6H), 2.39 (t, J=7.2 Hz, 6H), 2.25 (t, J=7.6 Hz, 6H), 1.68-1.51 (m, 18H), 1.50-1.39 (m, 6H), 1.35-1.16 (m, 102H), 0.87 (t, J=6.8 Hz, 18H). HRMS (ESI, m/z): [M+H]⁺ calcd. For C₉₀H₁₇₁NO₁₂, 1459.28740; found 1459.28864.

Compound L0131: ¹H NMR (400 MHz, Chloroform-d) δ 5.27-5.14 (m, 3H), 4.03 (t, K=6.8 Hz, 6H), 2.78-2.29 (m, 25H), 2.25 (t, J=7.6 Hz, 5H), 1.75-1.53 (m, 18H), 1.53-1.41 (m, 6H), 1.41-1.14 (m, 78H), 0.91-0.81 (m, 18H). HRMS (ESI, m/z): [M+H]⁺ calcd. For C₈₄H₁₅₉N₃O₁₂, 1403.19965; found 1403.19718.

Compound L0132: HRMS (ESI, m/z): [M+H]⁺ calcd. For C₅₉H₁₁₃NO₁₀, 996.84372; found 996.84448.

Compound L0133: ¹H NMR (400 MHz, Chloroform-d) δ 5.26-5.16 (m, 2H), 4.05 (t, J=6.8 Hz, 4H), 3.84-3.66 (m, 2H), 3.50 (dd, J=11.2, 4.0 Hz, 1H), 2.83-2.39 (m, 10H), 2.26 (t, J=7.6 Hz, 4H), 1.70-1.40 (m, 16H), 1.39-1.14 (m, 60H), 0.87 (t, J=6.8 Hz, 12H). HRMS (ESI, m/z): [M+H]⁺ calcd. For C₅₉H₁₁₃NO₁₀, 996.84372; found 996.84466.

Compound L0134: ¹H NMR (400 MHz, Chloroform-d) δ 5.20 (p, J=6.4 Hz, 2H), 4.03 (t, J=6.8 Hz, 4H), 3.59 (t, J=5.2 Hz, 2H), 2.83-2.42 (m, 10H), 2.25 (t, J=7.6 Hz, 4H), 1.79-1.49 (m, 20H), 1.39-1.20 (m, 60H), 0.86 (t, J=6.8 Hz, 12H). HRMS (ESI, m/z): [M+H]⁺ calcd. For C₆₀H₁₁₅NO₉, 994.86446; found 994.86665.

Compound L0135: ¹H NMR (400 MHz, Chloroform-d) δ 5.24-5.14 (m, 2H), 4.03 (t, J=6.8 Hz, 4H), 3.63 (t, J=6.4 Hz, 2H), 2.93-2.62 (m, 6H), 2.60-2.47 (m, 4H), 2.25 (t, J=7.6 Hz, 4H), 1.74-1.47 (m, 20H), 1.44-1.18 (m, 64H), 0.86 (t, J=6.8 Hz, 12H). HRMS (ESI, m/z): [M+H]⁺ calcd. For C₆₂H₁₁₉NO₉, 1022.89576; found 1022.89860.

Compound L0136: ¹H NMR (400 MHz, Chloroform-d) δ 5.26-5.15 (m, 2H), 4.04 (t, J=6.8 Hz, 4H), 3.60 (t, J=5.2 Hz, 2H), 2.73-2.64 (m, 2H), 2.63-2.45 (m, 8H), 2.26 (t, J=7.6 Hz, 4H), 1.72-1.43 (m, 16H), 1.40-1.19 (m, 60H), 0.87 (t, J=6.8 Hz, 12H). HRMS (ESI, m/z): [M+H]⁺ calcd. For C₅₈H₁₁₁NO₉, 966.83316; found 966.83601.

Compound L0137; ¹H NMR (400 MHz, Chloroform-d) δ 5.24-5.17 (m, 2H), 4.04 (t, J=6.7 Hz, 4H), 3.78 (t, J=5.1 Hz, 2H), 2.72-2.42 (m, 10H), 2.26 (t, J=7.5 Hz, 4H), 1.75-1.43 (m, 20H), 1.39-1.22 (m, 54H), 0.88 (q, J=6.7 Hz, 12H). HRMS (ESI, M/S): [M+H]⁺ calcd. For C₅₅H₁₀₅O₉N, 924.78621; found: 924.78892.

### Preparation of Compound L0138, Compound L0139 and Compound L0140

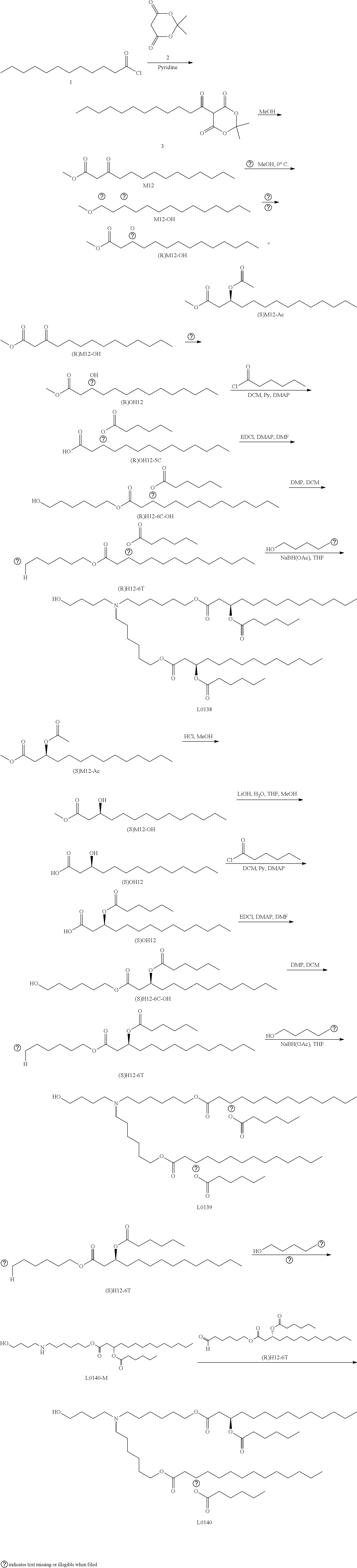

Synthesis of M12: To a solution of Cyclic (sub) isopropyl malonate (compound 3) (5.774 g, 40.00 mmol) in 50 mL of DCM, the solution of lauryl chloride (12.000 mL, 52.00 mmol) in 50 mL of DCM was injected at 0° C., and the mixture was stirred for 10 min. Then pyridine was added dropwise at 0° C. and stirred for 1 h. The mixture slowly returned to room temperature, and stirred overnight. Pyridine hydrochloride was removed by suction filtration with diatomite. The solution was diluted with EA and then washed with saturated NH₄Cl solution and saturated NaCl solution. The organic phase dried over anhydrous Na₂SO₄. The solution was filtered and the solvent was removed under reduced pressure. The residue was dissolved in MeOH (15 mL), then stirred at 80° C. for 12 h. The solution was cooled to room temperature and the solvent was removed under reduced pressure. The residue diluted with EA, washed with saturated NH₄Cl solution and saturated NaCl solution. The organic phase dried over anhydrous Na₂SO₄. The solution was filtered and the solvent was removed under reduced pressure. The residue was purified via CombiFlash system (PE/EA=1:1, Rf˜0.5) to give M12 as a colorless oil; 4.453 g, yield: 43.4%. HRMS (ESI, M/S): [M+H]⁺ calcd. For C₁₅H₂₉O₃, 257.21112; found: 257.21334. HRMS (ESI, M/S): [M+Na]⁺ calcd.For C₁₅H₂₈NaO₃, 279.19306; found: 279.19200.

Synthesis of M12-OH: To a solution of M12 (4.453 g, 17.37 mmol) in MeOH (180 mL), NaBH₄ (0.810 g, 20.84 mmol) was added slowly at 0° C. and stirred for 1.5 h. The mixture was quenched with saturated NH₄Cl solution and MeOH was removed under reduced pressure. The residue was dissolved with water and extracted EA. The organic phase dried over anhydrous Na₂SO₄. The solution was filtered and the solvent was removed under reduced pressure. The residue was purified via CombiFlash system (PE/EA=10:1, Rf˜0.5) to give M12 as a light yellow solid; 2.896 g, yield: 64.5%. HRMS (ESI, M/S): [M+H]⁺ calcd. For C₁₅H₃₁O₃, 259.22677; found: 259.22837. HRMS (ESI, M/S): [M+Na]⁺ calcd. For C₁₅H₃₀NaO₃, 281.20872; found: 281.21039.

Synthesis of (R)M12-OH and (S)M12-Ac: To a solution of M12-OH (2.038 g, 7.8 mmol) in toluene and vinyl acetate was was added CALB resin (1.017 g). The mixture was kept stirring for 48 h at 40° C. The reaction was filtered and the resin was washed with EA and PE. The solvent was removed under reduced pressure. The residue was purified via CombiFlash system (PE/EA=10:1) to give (R)M12-OH as a yellow oil and (S)M12-Ac as a bright yellow oil. (S)M12-Ac: HRMS (ESI, M/S): [M+Na]⁺ calcd. For C₁₇H₃₂O₃Na, 323.21928; found: 323.21842.

Synthesis of (R)OH12: To a solution of (R)M12-OH (0.888 g, 3.44 mmol) in THF and MeOH, 2.5M LiOH (6.330 mL, 15.82 mmol) was added. The mixture stirred at room temperature overnight. The mixture was quenched with 1M HCl and extracted with EA. The organic phase dried over anhydrous Na₂SO₄. The solution was filtered and the solvent was removed under reduced pressure. The residue was purified via CombiFlash system (PE/EA=1:1, Rf˜0.5) to give (R)OH12 as a light yellow solid; 0.675 g, yield: 80.3%. HRMS (ESI, M/S): [M+Na]⁺ calcd. For C₁₄H₂₈NaO₃, 267.19307; found: 267.19345.

Synthesis of (R)OH12-6C: To a solution of (R)OH12 (0.675 g, 2.76 mmol) in DCM, pyridine (0.367 mL, 4.01 mmol) and DMAP (0.034 g, 0.28 mmol) were added. Hexanoyl chloride (0.555 mL, 4.01 mmol) was injected dropwise at 0° C. The mixture was stirred for 1 day at room temperature. The solvent was removed under reduced pressure. The residue was dissolved with 1,4-dioxane and saturated NH₄Cl solution were added followed by stirring for 1 h. Then the mixture was extracted with DCM for three times. The organic phase dried over anhydrous Na₂SO₄. The solution was filtered and the solvent was removed under reduced pressure. The residue was purified via CombiFlash system (PE/EA=5:1, Rf˜0.5) to give (R)OH12-6C as a colorless oil; 0.527 g, yield: 55.8%. HRMS (ESI, M/S): [M+H]⁺ calcd. For C₂₀H₃₉O₄, 343.28429; found: 343.28486. HRMS (ESI, M/S): [M+Na]⁺ calcd. For C₂₀H₃₈NaO₄, 365.26623; found: 365.26702.

Synthesis of (R)H12-6C-OH: To solution of (R)OH12-6C (0.483 g, 1.41 mmol) in DMF (5 mL), DMAP (0.039 g, 0.28 mmol) and 1,6-hexanediol (0.840 g, 7.05 mmol) were added at 0° C. Then EDCI (0.402 g, 2.12 mmol) was added slowly. The mixture was stirred for 1 h at 0° C., then slowly returned to room temperature for overnight reacting. The mixture was quenched with a large amount of saturated NH₄Cl solution and extracted with DCM. The organic phase dried over anhydrous Na₂SO₄. The solution was filtered and the solvent was removed under reduced pressure. The residue was purified via CombiFlash system (PE/EA=5:1, Rf˜0.5) to give (R)H12-6C-OH as a light yellow solid; 0.307 g, yield: 49.5%. HRMS (ESI, M/S): [M+H]⁺ calcd. For C₂₆H₅₁O₅, 443.37310; found: 443.37269. HRMS (ESI, M/S): [M+Na]⁺ calcd. For C₂₆H₅₀NaO₅, 465.35505; found: 465.35529.

Synthesis of (R)H12-6T: To a solution of (R)H12-6C-OH (0.307 g, 0.69 mmol) in DCM (6 mL), NaHCO₃ (0.292 g, 3.47 mmol) was added. Then DMP (0.366 g, 0.87 mmol) was added at 0° C. and stirred for 5 min. The mixture slowly returned to room temperature and stirred for 2 h. After TLC detection, the reaction solution was carefully quenching with sodium thiosulfate. The mixture was extracted with EA three times. The organic phase dried over anhydrous Na₂SO₄. The solution was filtered and the solvent was removed under reduced pressure. The residue was purified via CombiFlash system (PE/EA=10:1, Rf˜0.5) to give (R)H12-6T as a colorless oil; 0.180 g, yield: 59.0%. HRMS (ESI, M/S): [M+H]⁺ calcd. For C₂₆H₄₉O₅, 441.35745; found: 441.35739. HRMS (ESI, M/S): [M+Na]⁺ calcd. For C₂₆H₄₈NaO₅, 463.33939; found: 463.33893.

Synthesis of L0138: To a mixture of (R)H12-6T (0.076 g, 0.17 mmol) and 4-amino-1-butanol (0.007 g, 0.007 mmol) in 2 mL of THF, NaBH₄ (0.042 g, 0.17 mmol) was added and stirred at room temperature overnight. The mixture was quenched with saturated NH₄Cl solution and extracted with DCM, then washed with saturated NaCl solution. The organic phase dried over anhydrous Na₂SO₄. The solution was filtered and the solvent was removed under reduced pressure. The residue was purified via CombiFlash system (DCM/MeOH=10:1, Rf˜0.5) to give L0138 as a yellow oil; 0.010 g, yield: 13.7%.

Synthesis of (S)M12-OH: To the solution of (S)M12-Ac (1.434 g, 4.77 mmol) in 1.5 mL of MeOH, the solution of HCl in 1,4-dioxane (4M, 1.5 mL) was added dropwise and stirred at room temperature overnight. The mixture was quenched with saturated NaHCO₃ solution and extracted with EA, then washed with saturated NaCl solution. The organic phase dried over anhydrous Na₂SO₄. The solution was filtered and the solvent was removed under reduced pressure. The residue was purified via CombiFlash system (PE/EA=7:1, Rf˜0.3) to give(S) M12-OH as a yellow cream; 1.012 g, yield: 82.1%. HRMS (ESI, M/S): [M+H]⁺ calcd. For C₁₅H₃₁O₃, 259.22677; found: 259.20221.

Synthesis of(S)OH12: To a solution of (S)M12-OH (1.111 g, 4.3 mmol) in THF and MeOH, 2.5M LiOH (7.9 mL, 19.8 mmol) was added. The mixture stirred at room temperature overnight. The mixture was quenched with 1M HCl and extracted with EA. The organic phase dried over anhydrous Na₂SO₄. The solution was filtered and the solvent was removed under reduced pressure. The residue was purified via CombiFlash system (PE/EA=1:1, Rf˜0.5) to give(S)OH12 as a light yellow solid; 0.892 g, yield: 84.9%. HRMS (ESI, M/S): [M+H]⁺ calcd. For C₁₄H₂₉O₃, 245.21112; found: 245.18109.

Synthesis of (S)OH12-6C: To a solution of(S)OH12 (0.892 g, 3.65 mmol) in DCM, pyridine (0.690 mL) and DMAP (0.045 g, 0.37 mmol) were added. Hexanoyl chloride (1.245 mL) was injected dropwise at 0° C. The mixture was stirred for 1 day at room temperature. The solvent was removed under reduced pressure. The residue was dissolved with 1,4-dioxane and saturated NH₄Cl solution were added followed by stirring for 1 h. Then the mixture was extracted with DCM for three times. The organic phase dried over anhydrous Na₂SO₄. The solution was filtered and the solvent was removed under reduced pressure. The residue was purified via CombiFlash system (PE/EA=5:1, Rf˜0.5) to give(S)OH12-6C as a colorless oil; 0.765 g, yield: 61.2%. HRMS (ESI, M/S): [M+H]⁺ calcd. For C₂₀H₃₉O₄, 343.28429; found: 343.31651.

Synthesis of (S)H12-6C-OH: To solution of (S)OH12-6C (0.765 g, 2.25 mmol) in DMF (10 mL), DMAP (0.029 g, 0.23 mmol) and 1,6-hexanediol (1.333 g, 11.25 mmol) were added at 0° C. Then EDCI (0.529 g, 2.70 mmol) was added slowly. The mixture was stirred for 1 h at 0° C., then slowly returned to room temperature for overnight reacting. The mixture was quenched with a large amount of saturated NH₄Cl solution and extracted with DCM. The organic phase dried over anhydrous Na₂SO₄. The solution was filtered and the solvent was removed under reduced pressure. The residue was purified via CombiFlash system (PE/EA=5:1, Rf˜0.5) to give(S) H12-6C-OH as a light yellow solid; 0.417 g, yield: 42.2% HRMS (ESI, M/S): [M+H]⁺ calcd. For C₂₆H₅₃O₅, 443.37310; found: 443.37303.

Synthesis of (S)H12-6T: To a solution of (S)H12-6C-OH (0.417 g, 0.94 mmol) in DCM (2 mL), NaHCO3 (0.395 g, 4.70 mmol) was added. Then DMP (0.502 g, 1.18 mmol) was added at 0° C. and stirred for 5 min. The mixture slowly returned to room temperature and stirred for 2 h. After TLC detection, the reaction solution was carefully quenching with sodium thiosulfate. The mixture was extracted with EA three times. The organic phase dried over anhydrous Na₂SO₄. The solution was filtered and the solvent was removed under reduced pressure. The residue was purified via CombiFlash system (PE/EA=10:1, Rf˜0.5) to give (R)H12-6T as a colorless oil; 0.206 g, yield: 49.6%. HRMS (ESI, M/S): [M+H]⁺ calcd. For C₂₆H₄₉O₅, 441.35745; found: 441.35798.

Synthesis of L0139: To a mixture of (S)H12-6T (0.074 g, 0.17 mmol) and 4-amino-1-butanol (0.006 g, 0.007 mmol) in 1 mL of THF, NaBH₄ (0.035 g, 0.17 mmol) was added and stirred at room temperature overnight. The mixture was quenched with saturated NH₄Cl solution and extracted with DCM, then washed with saturated NaCl solution. The organic phase dried over anhydrous Na₂SO₄. The solution was filtered and the solvent was removed under reduced pressure. The residue was purified via CombiFlash system (DCM/MeOH=10:1, Rf˜0.5) to give L0139 as a yellow oil; 0.021 g, yield: 33.2%.

Synthesis of L0140-M: To a solution of 4-amino-1-butanol (0.013 g, 0.15 mmol) in 1 mL of MeOH was added (S)H12-6T (0.070 g, 0.16 mmol), and stirred at room temperature for 1 h. Then NaBH₄ (0.007 g, 0.16 mmol) was added at 0° C. and the mixture was stirred at room temperature overnight. The mixture was quenched with saturated NH₄Cl solution and extracted with DCM, then washed with saturated NaCl solution. The organic phase dried over anhydrous Na₂SO₄. The solution was filtered and the solvent was removed under reduced pressure. The residue was purified via CombiFlash system (DCM/Ultra=2:1, Rf≈0.5) to give L0140-M as a yellow oil; 0.032 g, 42.7%. HRMS (ESI, M/S): [M+H]⁺ calcd. For C₃₀H₆₀NO₅, 514.44660; found: 514.44675.

Synthesis of L0140: To a mixture of L0140-M (0.032 g, 0.06 mmol) and (R)H12-6T (0.031 g, 0.072 mmol) in 1 mL of THF, NaBH (OAc) 3 (0.016 g, 0.072 mmol) was added and stirred at room temperature overnight. The mixture was quenched with saturated NH₄Cl solution and extracted with DCM, then washed with saturated NaCl solution. The organic phase dried over anhydrous Na₂SO₄. The solution was filtered and the solvent was removed under reduced pressure. The residue was purified via CombiFlash system (DCM/MeOH=10:1, Rf≈0.5) to give L0140 as a yellow oil; 0.015 g, yield: 25.7%.

Compound L0138: ¹H NMR (400 MHz, Chloroform-d) δ 5.21 (p, J=6.4 Hz, 2H), 4.04 (t, J=6.8 Hz, 4H), 3.62 (t, J=5.2 Hz, 2H), 2.88-2.63 (m, 6H), 2.62-2.46 (m, 4H), 2.26 (t, J=7.6 Hz, 4H), 1.71-1.52 (m, 20H), 1.38-1.24 (m, 52H), 0.91-0.86 (m, 12H). HRMS (ESI, M/S): [M+H]⁺ calcd. For C₅₆H₁₀₈O₉N, 938.80186; found: 980.8026.

Compound L0139: ¹H NMR (400 MHz, Chloroform-d) δ 5.20 (p, J=6.4 Hz, 2H), 4.03 (t, J=6.8 Hz, 4H), 3.62-3.49 (m, 2H), 2.71-2.39 (m, 10H), 2.25 (t, J=7.6 Hz, 4H), 1.73-1.47 (m, 20H), 1.37-1.21 (m, 52H), 0.87 (q, J=6.4 Hz, 12H). HRMS (ESI, M/S): [M+H]⁺ calcd. For C₅₆H₁₀₈O₉N, 938.80186; found: 938.80367.

Compound L0140: ¹H NMR (400 MHz, Chloroform-d) δ 5.20 (p, J=6.4 Hz, 2H), 4.04 (t, J=6.8 Hz, 4H), 3.61 (t, J=5.2 Hz, 2H), 2.83-2.44 (m, 10H), 2.26 (t, J=7.6 Hz, 4H), 1.79-1.54 (m, 20H), 1.37-1.22 (m, 52H), 0.88 (q, J=6.4 Hz, 12H). HRMS (ESI, M/S): [M+H]⁺ calcd. For C₅₆H₁₀₈O₉N, 938.8019; found: 938.8028.

### EXAMPLE 7: PREPARATION OF A NUCLEIC ACID NANOPARTICLE COMPLEX

A drug stock solution of nucleic acid (wherein, the nucleic acid may be FLUC mRNA, EGFP pDNA, EGFP siRNA or Spike mRNA) was diluted with a buffer solution to a nucleic acid concentration of 0.04 mg/mL (the buffer solution may be PBS buffer solution, citric acid-sodium citrate buffer solution, citric acid-disodium hydrogen phosphate buffer solution, HEPES buffer solution, sodium acetate buffer solution or Tris buffer solution with pH value of 3-6 and a concentration of 1-200 mM, preferably PBS buffer solution, citric acid-sodium citrate buffer solution with pH=4 and a concentrations of 10 mM, 20 mM and 50 mM; citric acid-sodium citrate buffer solution with pH=4 and a concentration of 50 mM was adopted in the embodiment) used as an aqueous phase.

Nucleic acid nanoparticle complexes were prepared according to the formulations listed in Table 1, the required PEG derivative, phospholipid, cholesterol analogue and lipid compound were taken out of a refrigerator, and warmed to room temperature, the PEG derivative, phospholipid, cholesterol analogue and lipid compound were weighed, and dissolved respectively in ethanol (the PEG derivative was dissolved in ethanol, the dissolution concentration was range from 1 mg/mL to 10 mg/mL; the phospholipid was dissolved in ethanol, the dissolution concentration was range from 1 mg/mL to 20 mg/mL; the cholesterol analogue was dissolved in ethanol, the dissolution concentration was range from 1 mg/mL to 20 mg/mL; the lipid compound was dissolved in ethanol, the dissolution concentration was range from 1 mg/mL to 30 mg/mL), ultrasonic dispersion was carried out to assist dissolution, corresponding volumes of the dissolved PEG derivative, phospholipid, cholesterol analogue and lipid compound were taken and mixed according to the proportion shown in Table 1, and an appropriate amount of ethanol was added and mixed (the added amount of ethanol was adaptively adjusted according to the component ratio of the nucleic acid nanoparticle components of each formulation and the ratio of the aqueous phase to the organic phase in subsequent operation), an ethanol solution containing PEG derivatives, phospholipids, cholesterol analogs and lipid compounds was formulated as the organic phase, after mixing the two phases rapidly by using a nanoparticle preparation instrument according to the volume ratio of the aqueous phase to the organic phase of 3:1 and the flow ratio of the aqueous phase to the organic phase of 3:1, the ethanol and water in the mixed solution were removed by a dialysis or ultrafiltration method, nuclease-free water was supplemented to ensure that the concentration of nucleic acid was 0.04 mg/mL, finally the formulations of the nucleic acid nanoparticle components shown in the table 1 were obtained, and stored at 2-8° C. for use later.

Formulation Rp.58 prepared using commercially available ionizable cationic lipid molecule SM-102 (CAS: 2089251-47-6) and formulation Rp.69 prepared using commercially available ionizable cationic lipid molecule ALC-0315 (CAS: 2036272-55-4) were used as controls.

TABLE 1
Formulations of nucleic acid nanoparticle complexes

| | | PEG derivative:phospho- | ratio of nitrogen |
|---|---|---|---|
| | | lipid:cholesterol | of lipid compound |
| | | analog:lipid compound | to phosphorus of |
| No. | Formulation information | (molar ratio) | nucleic acid |
| Rp.01 | DMG- | 2.50:16.00:16.50:65.00 | 15 |
| | PEG2000:DSPC:Chol:L0111:nu- | | |
| | cleic acid | | |
| Rp.02 | DMG- | 1.00:5.00:64.00:30.00 | 10 |
| | PEG2000:DSPC:Chol:L0111:nu- | | |
| | cleic acid | | |
| Rp.03 | mPEG- | 0.40:8.00:56.60:35.00 | 15 |
| | DSPE:DSPC:Chol:L0111:nu- | | |
| | cleic acid | | |
| Rp.04 | DMG- | 1.00:8.00:61.00:30.00 | 15 |
| | PEG2000:DSPC:Chol:L0111:nu- | | |
| | cleic acid | | |
| Rp.05 | DMG- | 1.00:16.00:34.50:48.50 | 10 |
| | PEG2000:DSPC:Chol:L0112:nu- | | |
| | cleic acid | | |
| Rp.06 | DMG- | 1.20:12.00:38.30:48.50 | 10 |
| | PEG2000:DSPC:Chol:L0112:nu- | | |
| | cleic acid | | |
| Rp.07 | DMG- | 1.70:9.00:40.80:48.50 | 10 |
| | PEG2000:DSPC:Chol:L0112:nu- | | |
| | cleic acid | | |
| Rp.08 | DMG- | 2.50:16.00:21.50:60.00 | 6 |
| | PEG2000:DSPC:Chol:L0112:nu- | | |
| | cleic acid | | |
| Rp.09 | DMG- | 1.20:9.00:47.80:42.00 | 10 |
| | PEG2000:DSPC:Chol:L0112:nu- | | |
| | cleic acid | | |
| Rp.10 | DMG- | 1.00:16.00:34.50:48.50 | 20 |
| | PEG2000:DSPC:Chol:L0113:nu- | | |
| | cleic acid | | |
| Rp.11 | DMG- | 2.50:8.00:41.00:48.50 | 25 |
| | PEG2000:DSPC:Chol:L0113:nu- | | |
| | cleic acid | | |
| Rp.12 | DMG- | 1.40:11.15:38.95:48.50 | 10 |
| | PEG2000:DSPC:Chol:L0115:nu- | | |
| | cleic acid | | |
| Rp.13 | DMG- | 1.50:8.00:25.50:65.00 | 20 |
| | PEG2000:DSPC:Chol:L0111:nu- | | |
| | cleic acid | | |
| Rp.14 | DMG- | 2.50:11.50:51.00:35.00 | 20 |
| | PEG2000:DSPC:Chol:L0111:nu- | | |
| | cleic acid | | |
| Rp.15 | DMG- | 3.20:12.00:29.80:55.00 | 20 |
| | PEG2000:DSPC:Chol:L0111:nu- | | |
| | cleic acid | | |
| Rp.16 | DMG- | 1.50:8.00:30.50:60.00 | 10 |
| | PEG2000:DSPC:Chol:L0112:nu- | | |
| | cleic acid | | |
| Rp.17 | DMG- | 1.00:5.00:62.00:32.00 | 6 |
| | PEG2000:DSPC:Chol:L0112:nu- | | |
| | cleic acid | | |
| Rp.18 | DMG- | 0.60:5.00:54.40:40.00 | 6 |
| | PEG2000:DSPC:Chol:L0112:nu- | | |
| | cleic acid | | |
| Rp.19 | DMG- | 1.00:8.00:53.00:38.00 | 6 |
| | PEG2000:DSPC:Chol:L0112:nu- | | |
| | cleic acid | | |
| Rp.20 | DMG- | 1.50:16.00:47.50:35.00 | 25 |
| | PEG2000:DSPC:Chol:L0113:nu- | | |
| | cleic acid | | |
| Rp.21 | DMG- | 1.63:12.99:45.38:40.00 | 10 |
| | PEG2000:DSPC:Chol:L0114:nu- | | |
| | cleic acid | | |
| Rp.22 | mPEG- | 1.30:8.00:55.70:35.00 | 15 |
| | STA:DSPC:Chol:L0111:nu- | | |
| | cleic acid | | |
| Rp.23 | mPEG- | 1.20:8.00:55.8:35.00 | 15 |
| | PS:DSPC:Chol:L0111:nu- | | |
| | cleic acid | | |
| Rp.24 | mPEG- | 1.30:8.00:55.70:35.00 | 15 |
| | DMPE:DSPC:Chol:L0111:nu- | | |
| | cleic acid | | |
| Rp.25 | mPEG- | 0.40:5.00:59.60:35.00 | 15 |
| | DSPE:DOPE:Chol:L0111:nu- | | |
| | cleic acid | | |
| Rp.26 | DMG- | 1.00:20.00:37.00:42.00 | 10 |
| | PEG2000:DPPC:Chol:L0112:nu- | | |
| | cleic acid | | |
| Rp.27 | DMG- | 1.00:25.00:34.00:40.00 | 19 |
| | PEG2000:PC:Chol:L0113:nu- | | |
| | cleic acid | | |
| Rp.28 | DMG- | 3.20:12.00:29.80:55.00 | 20 |
| | PEG2000:DSPC:Chol:L0111:nu- | | |
| | cleic acid | | |
| Rp.29 | DMG- | 1.00:8.00:53.00:38.00 | 6 |
| | PEG2000:DSPC:Chol:L0112:nu- | | |
| | cleic acid | | |
| Rp.30 | DMG- | 2.50:9.50:33.00:55.00 | 20 |
| | PEG2000:DSPC:Chol:L0111:nu- | | |
| | cleic acid | | |
| Rp.31 | mPEG- | 10.00:16.00:54.00:20.00 | 15 |
| | DPPE:DSPC:Chol:L0111:nu- | | |
| | cleic acid | | |
| Rp.32 | mPEG- | 3.00:30.00:42.00:25.00 | 20 |
| | DSPE:PC:Chol:L0111:nu- | | |
| | cleic acid | | |
| Rp.33 | DMG-PEG2000:DSPC:la- | 3.20:16.80:10.00:70.00 | 20 |
| | nosterol:L0111:nucleic | | |
| | acid | | |
| Rp.34 | DMG-PEG2000:DSPC:5α- | 3.00:17.00:25.00:55.00 | 20 |
| | cholestan-3β- | | |
| | ol:L0111:nucleic acid | | |
| Rp.35 | DMG-PEG2000:DSPC:er- | 3.20:16.80:15.00:65.00 | 20 |
| | gosterol:L0111:nu- | | |
| | cleic acid | | |
| Rp.36 | mPEG- | 4.20:11.00:70.00:14.80 | 10 |
| | DPPE:DOPE:Chol:L0111:nu- | | |
| | cleic acid | | |
| Rp.37 | mPEG- | 6.20:6.80:75.00:15.00 | 10 |
| | PS:DPPC:Chol:L0111:nu- | | |
| | cleic acid | | |
| Rp.38 | DMG- | 1.50:11.50:38.50:48.50 | 15 |
| | PEG2000:DSPC:Chol:L0113:nu- | | |
| | cleic acid | | |
| Rp.39 | DMG- | 1.40:11.15:38.95:48.50 | 6 |
| | PEG2000:DSPC:Chol:L0116:nu- | | |
| | cleic acid | | |
| Rp.40 | DMG- | 1.40:11.15:38.95:48.50 | 8 |
| | PEG2000:DSPC:Chol:L0117:nu- | | |
| | cleic acid | | |
| Rp.41 | DMG- | 1.40:11.15:38.95:48.50 | 30 |
| | PEG2000:DSPC:Chol:L0118:nu- | | |
| | cleic acid | | |
| Rp.42 | DMG- | 1.40:11.15:38.95:48.50 | 30 |
| | PEG2000:DSPC:Chol:L0119:nu- | | |
| | cleic acid | | |
| Rp.43 | DMG- | 1.40:11.15:38.95:48.50 | 10 |
| | PEG2000:DSPC:Chol:L0120:nu- | | |
| | cleic acid | | |
| Rp.44 | DMG- | 1.40:11.15:38.95:48.50 | 10 |
| | PEG2000:DSPC:Chol:L0121:nu- | | |
| | cleic acid | | |
| Rp.45 | mPEG- | 7.50:9.61:35.56:47.33 | 15 |
| | PS:DSPC:Chol:L0121:nu- | | |
| | cleic acid | | |
| Rp.46 | DMG- | 1.40:11.15:38.95:48.50 | 10 |
| | PEG2000:DSPC:Chol:L0122:nu- | | |
| | cleic acid | | |
| Rp.47 | DMG- | 1.40:11.15:38.95:48.50 | 10 |
| | PEG2000:DSPC:Chol:L0123:nu- | | |
| | cleic acid | | |
| Rp.48 | DMG- | 3.00:9.50:32.50:55.00 | 40 |
| | PEG2000:DSPC:Chol:L0111:nu- | | |
| | cleic acid | | |
| Rp.49 | DMG- | 3.00:9.50:32.50:55.00 | 50 |
| | PEG2000:DSPC:Chol:L0111:nu- | | |
| | cleic acid | | |
| Rp.50 | DMG- | 1.40:11.15:38.95:48.50 | 10 |
| | PEG2000:DSPC:Chol:L0124:nu- | | |
| | cleic acid | | |
| Rp.51 | DMG- | 1.40:11.15:38.95:48.50 | 10 |
| | PEG2000:DSPC:Chol:L0125:nu- | | |
| | cleic acid | | |
| Rp.52 | DMG- | 1.40:11.15:38.95:48.50 | 50 |
| | PEG2000:DSPC:Chol:L0126:nu- | | |
| | cleic acid | | |
| Rp.53 | DMG- | 0.95:7.58:26.47:65.00 | 50 |
| | PEG2000:DSPC:Chol:L0126:nu- | | |
| | cleic acid | | |
| Rp.54 | DMG- | 1.40:11.15:38.95:48.50 | 50 |
| | PEG2000:DSPC:Chol:L0127:nu- | | |
| | cleic acid | | |
| Rp.55 | DMG- | 1.40:11.15:38.95:48.50 | 40 |
| | PEG2000:DSPC:Chol:L0128:nu- | | |
| | cleic acid | | |
| Rp.56 | DMG- | 1.40:11.15:38.95:48.50 | 9 |
| | PEG2000:DSPC:Chol:L0129:nu- | | |
| | cleic acid | | |
| Rp.57 | DMG- | 1.40:11.15:38.95:48.50 | 9 |
| | PEG2000:DSPC:Chol:L0130:nu- | | |
| | cleic acid | | |
| Rp.58 | DMG- | 1.40:11.15:38.95:48.50 | 10 |
| | PEG2000:DSPC:Chol:SM-102:nu- | | |
| | cleic acid | | |
| Rp.59 | DMG- | 1.00:6.00:58.00:35.00 | 15 |
| | PEG2000:DSPC:Chol:L0131:nu- | | |
| | cleic acid | | |
| Rp.60 | DMG- | 1.40:11.15:38.95:48.50 | 5 |
| | PEG2000:DSPC:Chol:L0132:nu- | | |
| | cleic acid | | |
| Rp.61 | DMG- | 1.40:11.15:38.95:48.50 | 10 |
| | PEG2000:DSPC:Chol:L0133:nu- | | |
| | cleic acid | | |
| Rp.62 | DMG- | 1.40:11.15:38.95:48.50 | 10 |
| | PEG2000:DSPC:Chol:L0134:nu- | | |
| | cleic acid | | |
| Rp.63 | DMG- | 1.40:11.15:38.95:48.50 | 10 |
| | PEG2000:DSPC:Chol:L0135:nu- | | |
| | cleic acid | | |
| Rp.64 | DMG- | 1.40:11.15:38.95:48.50 | 10 |
| | PEG2000:DSPCChol:L0136:nu- | | |
| | cleic acid | | |
| Rp.65 | DMG- | 2.50:9.50:38.00:50.00 | 12 |
| | PEG2000:DSPC:Chol:L0111:nu- | | |
| | cleic acid | | |
| Rp.66 | DMG- | 2.50:9.50:38.00:50.00 | 12 |
| | PEG2000:DSPC:Chol:L0112:nu- | | |
| | cleic acid | | |
| Rp.67 | DMG- | 2.50:9.50:38.00:50.00 | 12 |
| | PEG2000:DSPC:Chol:L0114:nu- | | |
| | cleic acid | | |
| Rp.68 | DMG- | 2.50:9.50:38.00:50.00 | 12 |
| | PEG2000:DSPC:Chol:L0115:nu- | | |
| | cleic acid | | |
| Rp.69 | DMG- | 2.50:9.50:38.00:50.00 | 12 |
| | PEG2000:DSPC:Chol:ALC- | | |
| | 0315:nucleic acid | | |
| Rp.70 | DMG- | 2.10:7.98:47.90:42.02 | 10 |
| | PEG2000:DOPE:Chol:L0138:nu- | | |
| | cleic acid | | |
| Rp.71 | DMG- | 2.10:7.98:47.90:42.02 | 10 |
| | PEG2000:DOPE:Chol:L0139:nu- | | |
| | cleic acid | | |
| Rp.72 | DMG- | 2.10:7.98:47.90:42.02 | 10 |
| | PEG2000:DOPE:Chol:L0140:nu- | | |
| | cleic acid | | |
| Rp.73 | ALC- | 1.80:40.00:27.20:31.00 | 6 |
| | 0159:DSPC:Chol:L0137:nu- | | |
| | cleic acid | | |
| Rp.74 | DMG- | 3.00:6.00:43.00:48.00 | 8 |
| | PEG2000:DSPC:Chol:L0111:nu- | | |
| | cleic acid | | |
| Rp.75 | DMG- | 1.60:30.00:30.00:40.00 | 6.5 |
| | PEG2000:DSPC:Chol:L0111:nu- | | |
| | cleic acid | | |
| Rp.76 | DMG- | 1.60:35.00:35.00:30.00 | 6.5 |
| | PEG2000:DSPC:Chol:L0111:nu- | | |
| | cleic acid | | |
| Rp.77 | DMG- | 1.60:40.00:40.00:20.00 | 6.5 |
| | PEG2000:DSPC:Chol:L0111:nu- | | |
| | cleic acid | | |
| Rp.78 | ALC- | 1.80:40.00:27.20:31.00 | 6 |
| | 0159:DSPC:Chol:L0111:nu- | | |
| | cleic acid | | |
| Rp.79 | ALC- | 1.80:40.00:27.20:31.00 | 6 |
| | 0159:DSPC:Chol:L0131:nu- | | |
| | cleic acid | | |
| Rp.80 | ALC- | 1.80:40.00:27.20:31.00 | 6 |
| | 0159:DSPC:Chol:L0135:nu- | | |
| | cleic acid | | |
| Rp.81 | ALC-0159:DSPC:5α- | 1.80:40.00:27.20:31.00 | 6 |
| | cholestan-3β- | | |
| | ol:L0111:nucleic acid | | |
| Rp.82 | ALC-0159:DSPC:La- | 1.80:40.00:27.20:31.00 | 6 |
| | nosterol:L0111:nucleic acid | | |
| Rp.83 | STA- | 1.80:40.00:27.20:31.00 | 6 |
| | mPEG2000:DSPC:Chol:L0111:nu- | | |
| | cleic acid | | |
| Rp.84 | PS- | 1.80:40.00:27.20:31.00 | 6 |
| | mPEG2000:DSPC:Chol:L0111:nu- | | |
| | cleic acid | | |

Note:
the nitrogen-to-phosphorus ratio of lipid compound to nucleic acid in table 1 represents the ratio of the molar amount of ionizable nitrogen atoms in the lipid compound (i.e., the compound provided herein, such as at least one of compounds L0111-L0136, or ALC-0315 or SM-102) to the molar amount of phosphorus atoms in nucleic acid of the nucleic acid nanoparticle complex.

### EXAMPLE 8: CHARACTERIZATION OF THE NUCLEIC ACID NANOPARTICLE COMPLEXES OF THE INVENTION

Particle size and potential: nucleic acid nanoparticle complexes were prepared according to the method described in example 7, and dynamic light scattering particle size (size), surface Potential (Zeta Potential) and Polymer dispersity index (PDI) of the nucleic acid nanoparticle complex were tested at 25° C. by using a Malvern nanosizer (Malvern Zetasizer Nano ZSE).

The results were shown in Table 2, and indicate that the particle size of the nucleic acid nanoparticle complex is in the range of 68 nm to 210 nm, the nucleic acid nanoparticle complex has better dispersibility, and the surface charge of the nucleic acid nanoparticle complex is in the range of −12 mV to +26 mV.

Entrapment rate: the nucleic acid nanoparticle complex was prepared according to the preparation method described in example 7 by using FLuc-mRNA as model mRNA, and the encapsulation rate of each formulation on the mRNA was detected by using a Quant-iT RiboGreen RNA detection kit (ThermoFische company), the standard procedure of the Quant-iT RiboGreen RNA kit was referred in the specific method for measurement, and the results were shown in Table 2, wherein the formulations have good encapsulation effect on mRNA, and the entrapment rate is more than 85%.

TABLE 2
Characterization of nucleic acid nanoparticle complexes

| | | Particle | | Zeta | Entrapment |
|---|---|---|---|---|---|
| | | size | | potential | rate |
| No. | Formulation information | (nm) | PDI | (mV) | (%) |
| Rp.01 | DMG- | 104.07 ± 1.31 | 0.18 ± 0.01 | 15.60 ± 0.75 | — |
| | PEG2000:DSPC:Chol:L0111:FLuc- | | | | |
| | mRNA | | | | |
| Rp.02 | DMG- | 101.30 ± 0.17 | 0.08 ± 0.02 | 17.13 ± 0.21 | — |
| | PEG2000:DSPC:Chol:L0111:FLuc- | | | | |
| | mRNA | | | | |
| Rp.03 | mPEG- | 120.93 ± 0.74 | 0.11 ± 0.02 | 8.20 ± 0.33 | 85.74% |
| | DSPE:DSPC:Chol:L0111:FLuc- | | | | |
| | mRNA | | | | |
| Rp.04 | DMG- | 114.33 ± 1.04 | 0.05 ± 0.02 | 16.63 ± 0.72 | — |
| | PEG2000:DSPC:Chol:L0111:FLuc- | | | | |
| | mRNA | | | | |
| Rp.05 | DMG- | 99.20 ± 2.32 | 0.17 ± 0.00 | 12.67 ± 0.42 | — |
| | PEG2000:DSPC:Chol:L0112:FLuc- | | | | |
| | mRNA | | | | |
| Rp.06 | DMG- | 104.00 ± 0.26 | 0.18 ± 0.01 | 18.37 ± 0.75 | — |
| | PEG2000:DSPC:Chol:L0112:FLuc- | | | | |
| | mRNA | | | | |
| Rp.07 | DMG- | 125.27 ± 0.21 | 0.14 ± 0.01 | 10.60 ± 0.52 | — |
| | PEG2000:DSPC:Chol:L0112:FLuc- | | | | |
| | mRNA | | | | |
| Rp.08 | DMG- | 106.20 ± 1.31 | 0.15 ± 0.00 | 14.07 ± 0.67 | — |
| | PEG2000:DSPC:Chol:L0112:FLuc- | | | | |
| | mRNA | | | | |
| Rp.09 | DMG- | 123.20 ± 0.95 | 0.13 ± 0.02 | 10.93 ± 0.64 | — |
| | PEG2000:DSPC:Chol:L0112:FLuc- | | | | |
| | mRNA | | | | |
| Rp.10 | DMG- | 159.50 ± 1.51 | 0.19 ± 0.01 | 25.93 ± 0.71 | — |
| | PEG2000:DSPC:Chol:L0113:FLuc- | | | | |
| | mRNA | | | | |
| Rp.11 | DMG- | 149.63 ± 0.45 | 0.20 ± 0.01 | 11.07 ± 0.35 | 85.08% |
| | PEG2000:DSPC:Chol:L0113:FLuc- | | | | |
| | mRNA | | | | |
| Rp.12 | DMG- | 91.15 ± 1.00 | 0.13 ± 0.00 | 22.90 ± 0.82 | — |
| | PEG2000:DSPC:Chol:L0115:FLuc- | | | | |
| | mRNA | | | | |
| Rp.13 | DMG- | 104.33 ± 0.81 | 0.19 ± 0.01 | 11.37 ± 0.21 | — |
| | PEG2000:DSPC:Chol:L0111:FLuc- | | | | |
| | mRNA | | | | |
| Rp.14 | DMG- | 107.77 ± 0.35 | 0.20 ± 0.02 | −0.13 ± 1.90 | — |
| | PEG2000:DSPC:Chol:L0111:FLuc- | | | | |
| | mRNA | | | | |
| Rp.15 | DMG- | 123.20 ± 0.70 | 0.22 ± 0.01 | 12.07 ± 1.10 | — |
| | PEG2000:DSPC:Chol:L0111:FLuc- | | | | |
| | mRNA | | | | |
| Rp.16 | DMG- | 113.97 ± 2.36 | 0.13 ± 0.01 | 13.13 ± 1.27 | — |
| | PEG2000:DSPC:Chol:L0112:FLuc- | | | | |
| | mRNA | | | | |
| Rp.17 | DMG- | 131.07 ± 20.00 | 0.23 ± 0.00 | 13.67 ± 0.49 | — |
| | PEG2000:DSPC:Chol:L0112:FLuc- | | | | |
| | mRNA | | | | |
| Rp.18 | DMG- | 120.03 ± 1.35 | 0.12 ± 0.02 | 17.03 ± 0.70 | — |
| | PEG2000:DSPC:Chol:L0112:FLuc- | | | | |
| | mRNA | | | | |
| Rp.19 | DMG- | 117.00 ± 1.06 | 0.18 ± 0.04 | 14.33 ± 0.72 | 95.11% |
| | PEG2000:DSPC:Chol:L0112:FLuc- | | | | |
| | mRNA | | | | |
| Rp.20 | DMG- | 130.50 ± 1.41 | 0.16 ± 0.01 | 19.23 ± 0.55 | — |
| | PEG2000:DSPC:Chol:L0113:FLuc- | | | | |
| | mRNA | | | | |
| Rp.21 | DMG- | 110.80 ± 0.40 | 0.15 ± 0.02 | 17.30 ± 1.08 | 95.02% |
| | PEG2000:DSPC:Chol:L0114:FLuc- | | | | |
| | mRNA | | | | |
| Rp.22 | mPEG- | 191.20 ± 1.73 | 0.06 ± 0.02 | 18.40 ± 1.39 | — |
| | STA:DSPC:Chol:L0111:FLuc- | | | | |
| | mRNA | | | | |
| Rp.23 | mPEG- | 127.70 ± 0.40 | 0.09 ± 0.03 | 15.83 ± 2.24 | — |
| | PS:DSPC:Chol:L0111:FLuc- | | | | |
| | mRNA | | | | |
| Rp.24 | mPEG- | 209.90 ± 0.61 | 0.06 ± 0.01 | 14.90 ± 0.46 | — |
| | DMPE:DSPC:Chol:L0111:FLuc- | | | | |
| | mRNA | | | | |
| Rp.25 | mPEG- | 129.07 ± 0.47 | 0.12 ± 0.02 | 4.88 ± 1.02 | — |
| | DSPE:DOPE:Chol:L0111:FLuc- | | | | |
| | mRNA | | | | |
| Rp.26 | DMG- | 106.27 ± 1.62 | 0.16 ± 0.01 | 15.20 ± 0.36 | — |
| | PEG2000:DPPC:Chol:L0112:FLuc- | | | | |
| | mRNA | | | | |
| Rp.27 | DMG- | 127.43 ± 1.74 | 0.11 ± 0.02 | 15.33 ± 1.50 | — |
| | PEG2000:PC:Chol:L0113:FLuc- | | | | |
| | mRNA | | | | |
| Rp.28 | DMG- | 102.93 ± 0.76 | 0.24 ± 0.00 | 13.57 ± 0.80 | — |
| | PEG2000:DSPC:Chol:L0111:FLuc- | | | | |
| | mRNA | | | | |
| Rp.29 | DMG- | 111.97 ± 0.31 | 0.10 ± 0.01 | 12.13 ± 1.12 | — |
| | PEG2000:DSPC:Chol:L0112:FLuc- | | | | |
| | mRNA | | | | |
| Rp.30 | DMG- | 109.60 ± 2.01 | 0.22 ± 0.01 | 9.75 ± 0.27 | — |
| | PEG2000:DSPC:Chol:L0111:FLuc- | | | | |
| | mRNA | | | | |
| Rp.31 | mPEG- | 129.00 ± 1.14 | 0.21 ± 0.01 | 10.93 ± 0.49 | — |
| | DPPE:DSPC:Chol:L0111:FLuc- | | | | |
| | mRNA | | | | |
| Rp.32 | mPEG- | 88.34 ± 1.19 | 0.30 ± 0.03 | 7.71 ± 0.36 | — |
| | DSPE:PC:Chol:L0111:FLuc- | | | | |
| | mRNA | | | | |
| Rp.33 | DMG-PEG2000:DSPC:la- | 146.00 ± 1.08 | 0.15 ± 0.02 | 12.77 ± 0.83 | — |
| | nosterol:L0111:FLuc- | | | | |
| | mRNA | | | | |
| Rp.34 | DMG-PEG2000:DSPC:5α- | 111.60 ± 1.06 | 0.21 ± 0.01 | 11.00 ± 0.10 | — |
| | cholestan-3β- | | | | |
| | ol:L0111:FLuc-mRNA | | | | |
| Rp.35 | DMG-PEG2000:DSPC:er- | 152.97 ± 2.06 | 0.19 ± 0.01 | 9.55 ± 0.35 | — |
| | gosterol:L0111:FLuc- | | | | |
| | mRNA | | | | |
| Rp.36 | mPEG- | 134.53 ± 0.76 | 0.20 ± 0.02 | 11.80 ± 0.69 | — |
| | DPPE:DOPE:Chol:L0111:FLuc- | | | | |
| | mRNA | | | | |
| Rp.37 | mPEG- | 100.12 ± 0.63 | 0.17 ± 0.03 | 11.20 ± 0.20 | — |
| | PS:DPPC:Chol:L0111:FLuc- | | | | |
| | mRNA | | | | |
| Rp.38 | DMG- | 117.60 ± 0.30 | 0.19 ± 0.01 | 9.69 ± 1.23 | — |
| | PEG2000:DSPC:Chol:L0113:FLuc- | | | | |
| | mRNA | | | | |
| Rp.39 | DMG- | 112.47 ± 2.25 | 0.24 ± 0.00 | 12.40 ± 1.90 | 96.12% |
| | PEG2000:DSPC:Chol:L0116:FLuc- | | | | |
| | mRNA | | | | |
| Rp.40 | DMG- | 91.34 ± 0.83 | 0.24 ± 0.00 | 13.30 ± 0.30 | — |
| | PEG2000:DSPC:Chol:L0117:FLuc- | | | | |
| | mRNA | | | | |
| Rp.41 | DMG- | 187.60 ± 1.15 | 0.10 ± 0.03 | 8.68 ± 1.10 | — |
| | PEG2000:DSPC:Chol:L0118:FLuc- | | | | |
| | mRNA | | | | |
| Rp.42 | DMG- | 107.80 ± 1.05 | 0.14 ± 0.00 | 11.53 ± 1.02 | — |
| | PEG2000:DSPC:Chol:L0119:FLuc- | | | | |
| | mRNA | | | | |
| Rp.43 | DMG- | 138.83 ± 0.71 | 0.09 ± 0.02 | 8.07 ± 0.19 | — |
| | PEG2000:DSPC:Chol:L0120:FLuc- | | | | |
| | mRNA | | | | |
| Rp.44 | DMG- | 125.13 ± 0.68 | 0.13 ± 0.01 | 9.66 ± 1.68 | — |
| | PEG2000:DSPC:Chol:L0121:FLuc- | | | | |
| | mRNA | | | | |
| Rp.45 | mPEG- | 102.57 ± 0.81 | 0.15 ± 0.00 | 11.20 ± 1.59 | — |
| | PS:DSPC:Chol:L0121:FLuc- | | | | |
| | mRNA | | | | |
| Rp.46 | DMG- | 116.17 ± 0.29 | 0.20 ± 0.01 | 13.57 ± 0.70 | — |
| | PEG2000:DSPC:Chol:L0122:FLuc- | | | | |
| | mRNA | | | | |
| Rp.47 | DMG- | 91.50 ± 1.12 | 0.18 ± 0.02 | 15.70 ± 1.66 | — |
| | PEG2000:DSPC:Chol:L0123:FLuc- | | | | |
| | mRNA | | | | |
| Rp.48 | DMG- | 72.52 ± 0.68 | 0.19 ± 0.02 | 15.90 ± 1.37 | — |
| | PEG2000:DSPC:Chol:L0111:FLuc- | | | | |
| | mRNA | | | | |
| Rp.49 | DMG- | 68.91 ± 0.27 | 0.19 ± 0.01 | 16.70 ± 1.55 | — |
| | PEG2000:DSPC:Chol:L0111:FLuc- | | | | |
| | mRNA | | | | |
| Rp.50 | DMG- | 116.50 ± 0.30 | 0.17 ± 0.02 | 14.50 ± 0.95 | — |
| | PEG2000:DSPC:Chol:L0124:FLuc- | | | | |
| | mRNA | | | | |
| Rp.51 | DMG- | 88.66 ± 1.22 | 0.27 ± 0.01 | 16.93 ± 0.76 | — |
| | PEG2000:DSPC:Chol:L0125:FLuc- | | | | |
| | mRNA | | | | |
| Rp.52 | DMG- | 126.97 ± 0.91 | 0.11 ± 0.01 | 13.80 ± 2.35 | — |
| | PEG2000:DSPC:Chol:L0126:FLuc- | | | | |
| | mRNA | | | | |
| Rp.53 | DMG- | 174.37 ± 1.16 | 0.06 ± 0.01 | 15.90 ± 4.00 | — |
| | PEG2000:DSPC:Chol:L0126:FLuc- | | | | |
| | mRNA | | | | |
| Rp.54 | DMG- | 120.43 ± 0.60 | 0.10 ± 0.01 | 13.43 ± 1.65 | — |
| | PEG2000:DSPC:Chol:L0127:FLuc- | | | | |
| | mRNA | | | | |
| Rp.55 | DMG- | 125.97 ± 1.48 | 0.10 ± 0.01 | 13.00 ± 0.10 | — |
| | PEG2000:DSPC:Chol:L0128:FLuc- | | | | |
| | mRNA | | | | |
| Rp.56 | DMG- | 111.60 ± 0.79 | 0.15 ± 0.02 | 3.84 ± 1.41 | — |
| | PEG2000:DSPC:Chol:L0129:FLuc- | | | | |
| | mRNA | | | | |
| Rp.57 | DMG- | 117.50 ± 0.79 | 0.11 ± 0.01 | 11.07 ± 0.93 | — |
| | PEG2000:DSPC:Chol:L0130:FLuc- | | | | |
| | mRNA | | | | |
| Rp.58 | DMG- | 78.88 ± 0.72 | 0.17 ± 0.01 | 12.50 ± 0.96 | — |
| | PEG2000:DSPC:Chol:SM- | | | | |
| | 102:FLuc-mRNA | | | | |
| Rp.59 | DMG- | 90.52 ± 0.37 | 0.10 ± 0.03 | 11.00 ± 0.87 | — |
| | PEG2000:DSPC:Chol:L0131:FLuc- | | | | |
| | mRNA | | | | |
| Rp.60 | DMG- | 102.27 ± 0.76 | 0.16 ± 0.01 | 11.17 ± 0.91 | — |
| | PEG2000:DSPC:Chol:L0132:FLuc- | | | | |
| | mRNA | | | | |
| Rp.61 | DMG- | 89.16 ± 0.72 | 0.19 ± 0.01 | 12.93 ± 1.50 | — |
| | PEG2000:DSPC:Chol:L0133:FLuc- | | | | |
| | mRNA | | | | |
| Rp.62 | DMG- | 72.34 ± 1.89 | 0.18 ± 0.02 | 9.40 ± 0.53 | — |
| | PEG2000:DSPC:Chol:L0134:FLuc- | | | | |
| | mRNA | | | | |
| Rp.63 | DMG- | 100.61 ± 1.29 | 0.22 ± 0.01 | 10.53 ± 0.68 | — |
| | PEG2000:DSPC:Chol:L0135:FLuc- | | | | |
| | mRNA | | | | |
| Rp.64 | DMG- | 85.92 ± 1.01 | 0.21 ± 0.01 | 12.27 ± 0.31 | — |
| | PEG2000:DSPC:Chol:L0136:FLuc- | | | | |
| | mRNA | | | | |
| Rp.65 | DMG- | 80.97 ± 0.18 | 0.11 ± 0.01 | −2.63 ± 1.03 | — |
| | PEG2000:DSPC:Chol:L0111:FLuc- | | | | |
| | mRNA | | | | |
| Rp.66 | DMG- | 97.01 ± 1.59 | 0.13 ± 0.02 | 7.84 ± 0.32 | — |
| | PEG2000:DSPC:Chol:L0112:FLuc- | | | | |
| | mRNA | | | | |
| Rp.67 | DMG- | 82.60 ± 0.83 | 0.19 ± 0.01 | 11.00 ± 0.36 | — |
| | PEG2000:DSPC:Chol:L0114:FLuc- | | | | |
| | mRNA | | | | |
| Rp.68 | DMG- | 93.32 ± 0.77 | 0.07 ± 0.01 | 9.40 ± 0.73 | — |
| | PEG2000:DSPC:Chol:L0115:FLuc- | | | | |
| | mRNA | | | | |
| Rp.69 | DMG-PEG2000:DSPC:Chol:ALC- | 68.39 ± 0.43 | 0.12 ± 0.01 | −11.40 ± 0.10 | — |
| | 0315:FLuc-mRNA | | | | |
| Rp.70 | DMG- | 92.90 ± 0.42 | 0.08 ± 0.01 | 3.13 ± 2.55 | 96.53% |
| | PEG2000:DOPE:Chol:L0138:nu- | | | | |
| | cleic acid | | | | |
| Rp.71 | DMG- | 89.93 ± 0.65 | 0.09 ± 0.02 | 5.53 ± 1.99 | 97.02% |
| | PEG2000:DOPE:Chol:L0139:nu- | | | | |
| | cleic acid | | | | |
| Rp.72 | DMG- | 95.96 ± 1.20 | 0.08 ± 0.01 | 7.38 ± 0.41 | 97.09% |
| | PEG2000:DOPE:Chol:L0140:nu- | | | | |
| | cleic acid | | | | |
| Rp.73 | ALC- | 83.58 ± 0.51 | 0.13 ± 0.01 | −5.28 ± 0.58 | 94.50% |
| | 0159:DSPC:Chol:L0137:nu- | | | | |
| | cleic acid | | | | |
| Rp.74 | DMG- | 101.82 ± 3.83 | 0.02 ± 0.01 | 4.75 ± 0.84 | 85.10% |
| | PEG2000:DSPC:Chol:L0111:nu- | | | | |
| | cleic acid | | | | |
| Rp.75 | DMG- | 107.77 ± 1.61 | 0.14 ± 0.02 | −2.41 ± 0.76 | 86.33% |
| | PEG2000:DSPC:Chol:L0111:nu- | | | | |
| | cleic acid | | | | |
| Rp.76 | DMG- | 105.47 ± 0.93 | 0.14 ± 0.03 | 0.22 ± 0.71 | 87.52% |
| | PEG2000:DSPC:Chol:L0111:nu- | | | | |
| | cleic acid | | | | |
| Rp.77 | DMG- | 101.17 ± 0.06 | 0.15 ± 0.01 | −0.22 ± 0.39 | 85.72% |
| | PEG2000:DSPC:Chol:L0111:nu- | | | | |
| | cleic acid | | | | |
| Rp.78 | ALC- | 81.75 ± 0.48 | 0.15 ± 0.01 | −6.37 ± 0.25 | 92.34% |
| | 0159:DSPC:Chol:L0111:nu- | | | | |
| | cleic acid | | | | |
| Rp.79 | ALC- | 88.99 ± 0.10 | 0.05 ± 0.01 | −8.57 ± 1.08 | 83.00% |
| | 0159:DSPC:Chol:L0131:nu- | | | | |
| | cleic acid | | | | |
| Rp.80 | ALC- | 80.39 ± 0.59 | 0.11 ± 0.02 | −7.06 ± 1.19 | 94.31% |
| | 0159:DSPC:Chol:L0135:nu- | | | | |
| | cleic acid | | | | |
| Rp.81 | ALC-0159:DSPC:5α- | 90.66 ± 0.34 | 0.14 ± 0.02 | −5.32 ± 0.36 | 90.39% |
| | cholestan-3β- | | | | |
| | ol:L0111:nucleic acid | | | | |
| Rp.82 | ALC-0159:DSPC:La- | 80.66 ± 0.78 | 0.14 ± 0.02 | −4.26 ± 0.63 | 88.13% |
| | nosterol:L0111:nucleic acid | | | | |
| Rp.83 | STA- | 152.37 ± 2.32 | 0.10 ± 0.03 | −7.59 ± 0.61 | 79.63% |
| | mPEG2000:DSPC:Chol:L0111:nu- | | | | |
| | cleic acid | | | | |
| Rp.84 | PS- | 97.50 ± 0.83 | 0.10 ± 0.01 | −7.25 ± 0.95 | 84.44% |
| | mPEG2000:DSPC:Chol:L0111:nu- | | | | |
| | cleic acid | | | | |

### EXAMPLE 9: IN VITRO CELL TRANSFECTION EXPERIMENTS AND CYTOTOXICITY INVESTIGATION OF NUCLEIC ACID NANOPARTICLE COMPLEXES

1) Experiments of in vitro transfecting nucleic acid nanoparticle complex loaded with FLUC-mRNA into DC2.4 (mouse dendritic cells), 4T1 (mouse breast cancer cells), 293T (human kidney epithelial cells) and HeLa (human cervical cancer cells):

The cell suspension in the logarithmic growth phase was subpackaged into a 96-well plate at the density of 4×10⁴ cells per well, and the plate was placed into an incubator at 37° C. and 5% CO₂ for static culture. After 24 h, the cell well plate was taken out, the complete medium was sucked out, and 180 μL opti-MEM was added. Nucleic acid nanoparticle complexes were prepared according to the preparation methods of various formulations of FLuc mRNA described in Example 7. The mixtures of nucleic acid nanoparticle complexes were diluted with PBS respectively, after standing still, and 200 ng of FLuc mRNA containing nucleic acid nanoparticle complex was added to each well. 4 wells were repeated for each sample. After 4 h of administration, the culture medium in the 96-well plate was replaced with complete medium. And continuing to culture for 24 hours, the complete culture medium was sucked out, the 96-well plate was rinsed with PBS (phosphate buffer solution), the D-Luciferin working solution was added, the 96-well plate was continued to be cultured in an incubator at 37° C. for 5 min, and the fluorescence expression intensity of the FLuc-mRNA was tested by imaging testing on an Omega-FLuostar microplate reader. Lipofectamine2000 (Invitrogen) was used as a positive control according to the manufacturer's instructions. Relative transfection efficiency was calculated as follows:

$$relative transfection efficiency ( \% ) = fluorescence intensity of nucleic acid nanoparticle complex transfected cells / fluorescence intensity of Lipo 2000 transfected cells \times 100 \%$$

Results: the transfection efficiency of FLuc mRNA of partial formulations in DC2.4 (mouse dendritic cells), 4T1 (mouse breast cancer cells), 293T (human renal epithelial cells), and HeLa (human cervical cancer cells) cells were as shown in table 3. Conclusion: Rp.03, Rp.09, Rp.14, Rp.15, Rp.21, Rp.33, Rp.34, Rp.35, Rp.39 and Rp.54 have better transfection effect.

TABLE 3
Relative transfection efficiency of Fluc-mRNA of various
formulations of example 8 in DC2.4, 4T1, 293T, HeLa cells
4T1 cells

| | | | |
|---|---|---|---|
| Lipo2000 | 100% | Rp.15 | 23% |
| Rp.03 | 44% | Rp.33 | 18% |
| Rp.09 | 18% | Rp.35 | 18% |
| Rp.14 | 26% | Rp.39 | 25% |

DC2.4 cells

| | | | |
|---|---|---|---|
| Lipo2000 | 100% | Rp.21 | 18% |
| Rp.03 | 69% | Rp.33 | 47% |
| Rp.09 | 24% | Rp.34 | 22% |
| Rp.14 | 27% | Rp.35 | 25% |
| Rp.15 | 27% | Rp.54 | 30% |

293T cells

| | | | |
|---|---|---|---|
| Lipo2000 | 100% | Rp.09 | 25% |
| Rp.03 | 26% | Rp.39 | 26% |

HeLa cells

| | | | |
|---|---|---|---|
| Lipo2000 | 100% | Rp.21 | 10% |
| Rp.03 | 18% | Rp.33 | 13% |
| Rp.09 | 10% | Rp.54 | 12% |

2) Toxicity experiments of transfection of nucleic acid nanoparticle complex loaded with FLuc-mRNA into DC2.4 (mouse dendritic cells), 4T1 (mouse breast cancer cells), 293T (human renal epithelial cells) and HeLa (human cervical cancer cells) in vitro:

The cell suspension in logarithmic growth phase was subpackaged into a 96-well plate at the density of 4×10⁴ cells per well, and was placed into an incubator of 37° C. and 5% CO₂ for static culture. After 24 h, the cell well plate was taken out, the complete medium was sucked out, and opti-MEM was added. Nucleic acid nanoparticle complexes were prepared by using FLuc-mRNA according to the preparation methods of various formulations in example 7, respectively, the mixture of nucleic acid nanoparticle complexes was diluted with PBS and then stood, nucleic acid nanoparticle complex containing 200 ng FLuc-mRNA was added into each well, and 4 wells were repeated for each sample. After 4 h of administration, the medium in 96-well plate was sucked out and replaced with complete medium. And the plate was cultured continuously for 48 hours, the complete culture medium was sucked out, the plate was rinsed with PBS once, the cell well without the formulation was as a negative control and CCK-8 culture medium well without cells was as a blank control, adding 90 μL of serum-free culture medium and 10 μL of CCK-8 mixed solution into each well, and continuously incubating in the incubator for 2-4 hours. Absorbance at 450 nm was measured by using an Omega-Fluostar microplate reader. Cell viability calculation formula:

$$Cell viability * \% = [ A ( drug ) - A ( blank ) ] / A ( no drug ) - A ( blank ) ] \times 100 \% ;$$

A (drug): absorbance of each well within DC2.4 cells, formulation solution and CCK-8 solution;
A (blank): absorbance of each well within CCK-8 solution;
A (no drug): absorbance of each well within DC2.4 cells and CCK-8 solution;
Cell viability: Cell proliferation activity or cell toxicity activity.

The results are shown in Table 4. Conclusion: The results indicate that the survival rates of cells are all above 80%, indicating that the nucleic acid nanoparticle complexes provided herein with various formulations have no significant cytotoxicity and good biocompatibility, which can be used for subsequent animal experiments in vivo.

TABLE 4
The survival rate of cells treated with
various formulations of Example 8
4T1

| | | | | | | | |
|---|---|---|---|---|---|---|---|
| Blank | 100% | Rp.12 | 107% | Rp.28 | 110% | Rp.34 | 104% |
| Rp.03 | 102% | Rp.14 | 109% | Rp.29 | 111% | Rp.35 | 109% |
| Rp.04 | 114% | Rp.15 | 98% | Rp.31 | 105% | Rp.36 | 103% |
| Rp.09 | 109% | Rp.20 | 112% | Rp.32 | 104% | Rp.37 | 133% |
| Rp.11 | 108% | Rp.21 | 101% | Rp.33 | 100% | Rp.47 | 111% |

DC2.4

| | | | | | | | |
|---|---|---|---|---|---|---|---|
| Blank | 100% | Rp.11 | 99% | Rp.28 | 81% | Rp.37 | 81% |
| Rp.04 | 100% | Rp.12 | 92% | Rp.29 | 86% | Rp.39 | 84% |
| Rp.03 | 101% | Rp.19 | 97% | Rp.31 | 92% | Rp.45 | 89% |
| Rp.09 | 106% | Rp.22 | 107% | Rp.32 | 88% | Rp.54 | 96% |

293T

| | | | | | | | |
|---|---|---|---|---|---|---|---|
| Blank | 100% | Rp.12 | 97% | Rp.31 | 88% | Rp.37 | 101% |
| Rp.03 | 91% | Rp.21 | 89% | Rp.33 | 89% | Rp.39 | 88% |
| Rp.09 | 108% | Rp.28 | 101% | Rp.34 | 86% | Rp.45 | 104% |
| Rp.11 | 122% | Rp.29 | 91% | Rp.36 | 90% | Rp.54 | 97% |

HeLa

| | | | | | | | |
|---|---|---|---|---|---|---|---|
| Blank | 100% | Rp.12 | 93% | Rp.22 | 95% | Rp.36 | 94% |
| Rp.03 | 95% | Rp.14 | 82% | Rp.28 | 96% | Rp.37 | 100% |
| Rp.04 | 106% | Rp.15 | 90% | Rp.29 | 93% | Rp.39 | 95% |
| Rp.09 | 114% | Rp.19 | 111% | Rp.31 | 91% | Rp.45 | 98% |
| Rp.11 | 104% | Rp.20 | 90% | Rp.32 | 90% | Rp.47 | 99% |

3) Experiment of transfection of nucleic acid nanoparticle complexes loaded with EGFP-pDNA into DC2.4 (mouse dendritic cells) and 4T1 (mouse breast cancer cells) in vitro:

The cell suspension in logarithmic growth phase was subpackaged into a 96-well plate at the density of 4×10⁴ cells per well, and was placed into an incubator of 37° C. and 5% CO₂ for static culture. After 24 h, the cell well plate was taken out, the complete medium was sucked out, and opti-MEM was added. Nucleic acid nanoparticle complexes were prepared by using EGFP-pDNA according to the preparation methods of various formulations in example 7, respectively, the mixture of nucleic acid nanoparticle complexes was diluted with PBS and then stood, nucleic acid nanoparticle complex containing 200 ng FLuc-mRNA was added into each well, and 4 wells were repeated for each sample. After 4 h of administration, the medium in 96-well plate was sucked out and replaced with complete medium. And the plate was cultured continuously for 24 hours, the cells were digested and collected, the fluorescence intensity of FITC channel of living cells was detected for each well using a flow cytometer, and the geometric mean of fluorescence intensity of duplicate wells of EGFP-positive cells was calculated. Lipofectamine2000 (Invitrogen) was used as a positive control according to the manufacturer's instructions. Relative transfection efficiency was calculated as follows:

$$Relative transfection efficiency ( \% ) = geometric mean of fluorescence intensity of cells transfected with nucleic acid nanoparticle complexes / geometric mean of fluorescence intensity of cells transfected with Lipo 2000 \times 100 \%$$

The results are shown in Table 5.

Conclusion: the results indicate that the nucleic acid nanoparticle complex loaded with EGFP-pDN A shows better expression quantity at the cellular level.

TABLE 5
Relative transfection efficiency of EGFP-pDNA of various
different formulations of example 7 in DC2.4, 4T1 cells

| | |
|---|---|
| 4T1 cells | DC2.4 cells |

| | | | |
|---|---|---|---|
| Lipo2000 | 100% | Lipo2000 | 100% |
| Rp.03 | 35% | Rp.34 | 22% |
| Rp.45 | 28% | Rp.37 | 70% |

4) Transfection of nucleic acid nanoparticle complexes loaded with EGFP-siRNA (taking EGFP-siRNA as model siRNA) into a HeLa-EGFP cell (a polyclonal cell strain stably expressing EGFP fluorescent protein) in vitro to silence gene expression experiments:

The cell suspension in logarithmic growth phase was subpackaged into a 24-well plate at the density of 1×10⁵ cells per well, and was placed into an incubator of 37° C. and 5% CO₂ for static culture. After 24 h, nucleic acid nanoparticle complexes were prepared by using EGFP-siRNA according to the preparation methods of various formulations in example 7, respectively, the mixture of nucleic acid nanoparticle complexes was diluted with PBS and then stood, nucleic acid nanoparticle complex containing 160 ng EGFP-siRNA was added into each well, and 3 wells were repeated for each sample. After 4 h of administration, the medium in 96-well plate was sucked out and replaced with complete medium. And the plate was cultured continuously for 18-24 hours, the cells were digested and collected, the fluorescence intensity of FITC channel of living cells was detected for each well using a flow cytometer, and the geometric mean of fluorescence intensity of duplicate wells of EGFP-positive cells was calculated. The silencing efficiency (i.e., percent decrease in mean fluorescence intensity of cells) of siRNA was calculated as follows:

$$Silencing efficiency of siRNA ( \% ) = ( geometric mean of fluorescence intensity of cells not transfected with nucleic acid nanoparticle complex - geometric mean of fluorescence intensity of cells transfected with nucleic acid nanoparticle complex ) / geometric mean of fluorescence intensity of cells not transfected with nucleic acid nanoparticle complex \times 100 \%$$

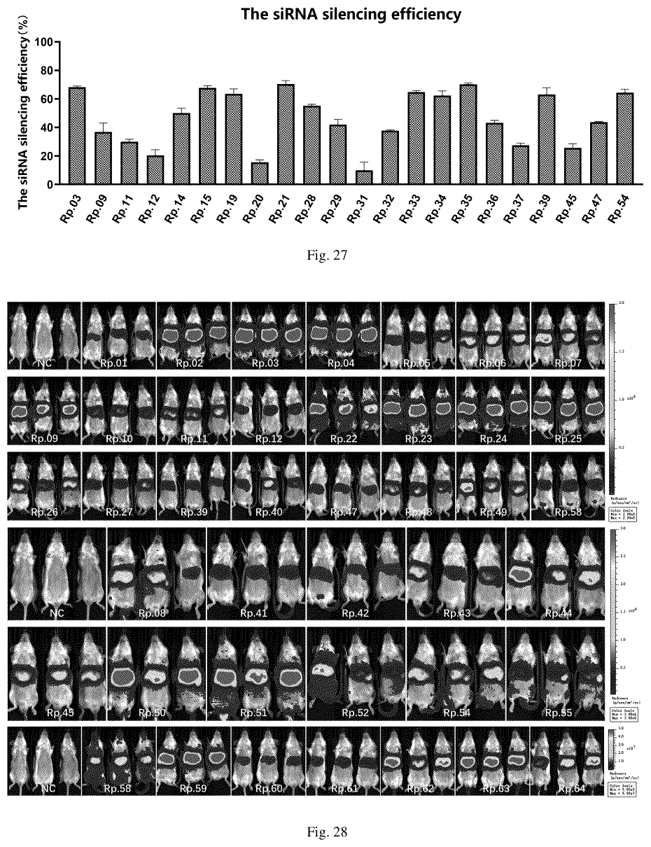

The results are shown in FIG. 27. Conclusion: the results indicate that the nucleic acid lipid compounds loaded with EGFP-siRNA show better gene silencing efficiency in cell transfection, where the silencing efficiency of delivery of siRNA by Rp.03, Rp. 14, Rp. 15, Rp. 19, Rp.21, Rp.28, Rp.33, Rp.34, Rp.35, Rp.39 and Rp.54 in cells exceeds 50%, better than other formulations.

### EXAMPLE 10: TRANSFECTION OF NUCLEIC ACID NANOPARTICLE COMPOUND IN VIVO OF MOUSE BY FLUORESCENCE IMAGING OF SMALL ANIMALS

Three BALB/c mice per group, nucleic acid nanoparticle complexes were prepared by using FLUC-mRNA as a model mRNA according to the preparation method described in example 7. 75 μL of nucleic acid nanoparticle complex containing 3 μg FLuc-mRNA was injected to each mouse of experimental groups using an insulin syringe needle. NC means blank control group, 75 μL of PBS buffer was injected using an insulin syringe needle. When the administration mode is intravenous injection, the injection site is tail vein of mouse. When the administration mode is intraperitoneal injection, the injection position is the abdominal cavity of a mouse. When the administration mode is intramuscular injection, the injection site is the thigh muscle of the mouse. When the administration mode is hypodermic injection, the injection site is hypodermic on the back of the mouse. Detection was carried out after 3 hours of intravenous injection administration, detection was carried out 6 hours of intraperitoneal injection administration, detection was carried out after 24 hours of intramuscular injection administration and detection was carried outafter 24 hours of hypodermic injection administration, a proper amount of substrate D-Luciferin was diluted with PBS to prepare a solution with the concentration of 15 mg/mL, the solution was kept out of the sun for standby, each mouse was injected with 200 μL of substrate though the abdominal cavity, placed in a mouse anesthesia box, the ventilation valve was opened, and isoflurane was released to anesthetize the mouse. After 5 min of substrate injection, mice were subjected to whole body in vivo imaging bioluminescence image detection using a small animal in vivo imaging system (Perkinelmer, IVIS Lumina Series III). A mouse bioluminescence image was taken.

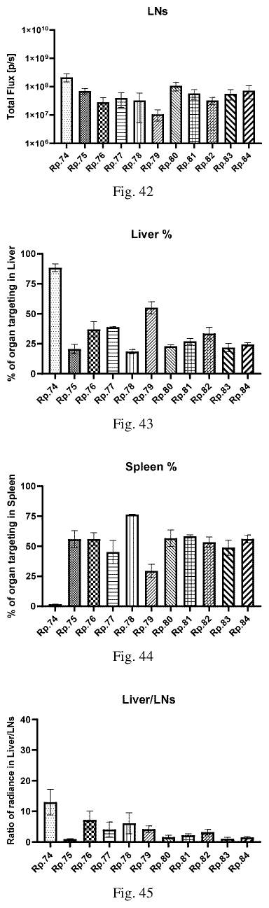

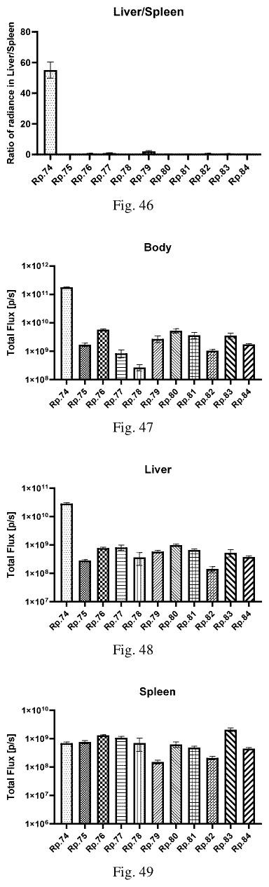

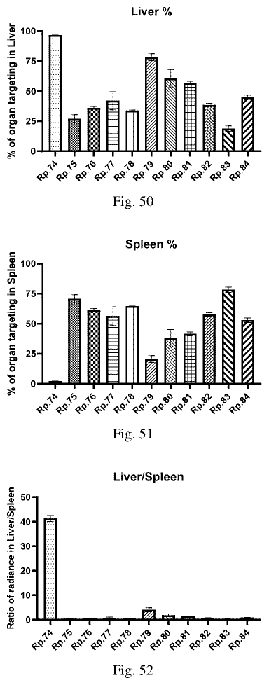

As a result, as shown in FIGS. 28 to 31, and FIGS. 37 to 52, the experimental group of nucleic acid nanoparticle complexes showed luciferase expression in the whole-body in vivo imaging, and the higher the fluorescence intensity, the more luciferase expression. Conclusion: the experiment group nucleic acid nanoparticle complex loaded with FLuc-mRNA has better luciferase expression in a mouse body.

As shown in FIG. 28, in the experimental group of intravenous administration, the effective expression was observed in mice after administration of all formulation. The luciferases expressions of the fomulations Rp.02, Rp.03, Rp.04, Rp.06, Rp.07, Rp.09, Rp.22, Rp.23, Rp.24, Rp.25, Rp.26, Rp.40, Rp.48, Rp.49, Rp.59, Rp.62, Rp.63, Rp.64 and Rp.65 were superior to other formulations and were obviously higher than the formulation Rp.58 prepared by the commercial cationic lipid SM-102.

As shown in FIG. 29, in the experimental group of intraperitoneal injection administration, the effective expression was observed in mice after administration of all formulations. The nucleic acid nanoparticle complex formulation in the experimental group has obvious effect.

As shown in FIG. 30, in the experimental group of intramuscular injection administration, the effective expression was observed in mice after administration of all formulations. The luciferases expressions of the formulations Rp.01, Rp.12, Rp. 13, Rp.14, Rp.15, Rp. 17, Rp.18, Rp. 19, Rp.30, Rp.48 and Rp.49 were superior to other formulations, and were obviously higher than the formulation Rp.58 prepared by the commercial cationic lipid SM-102.

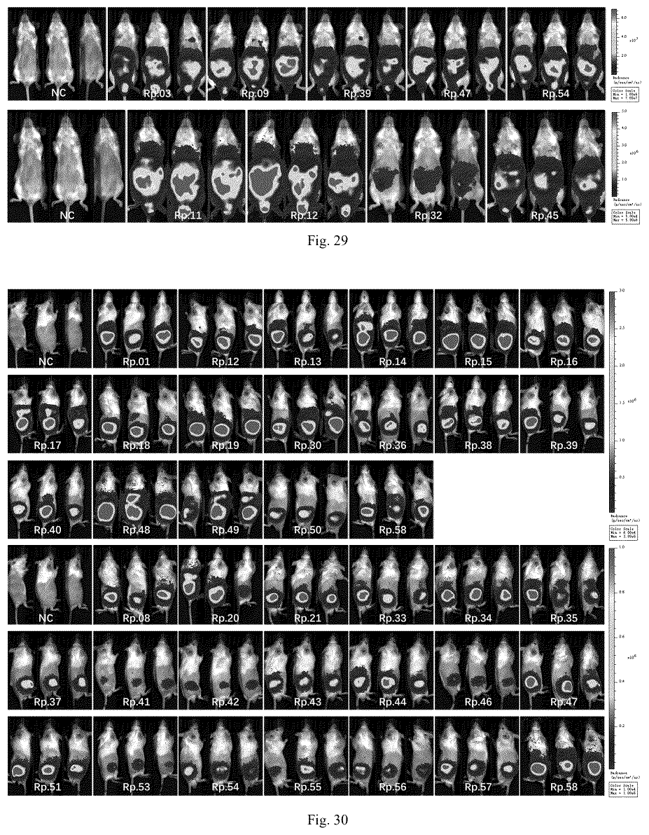

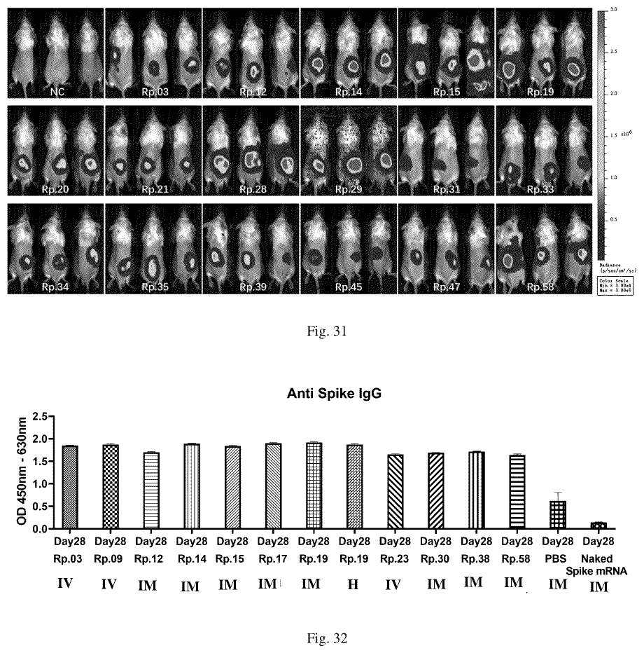

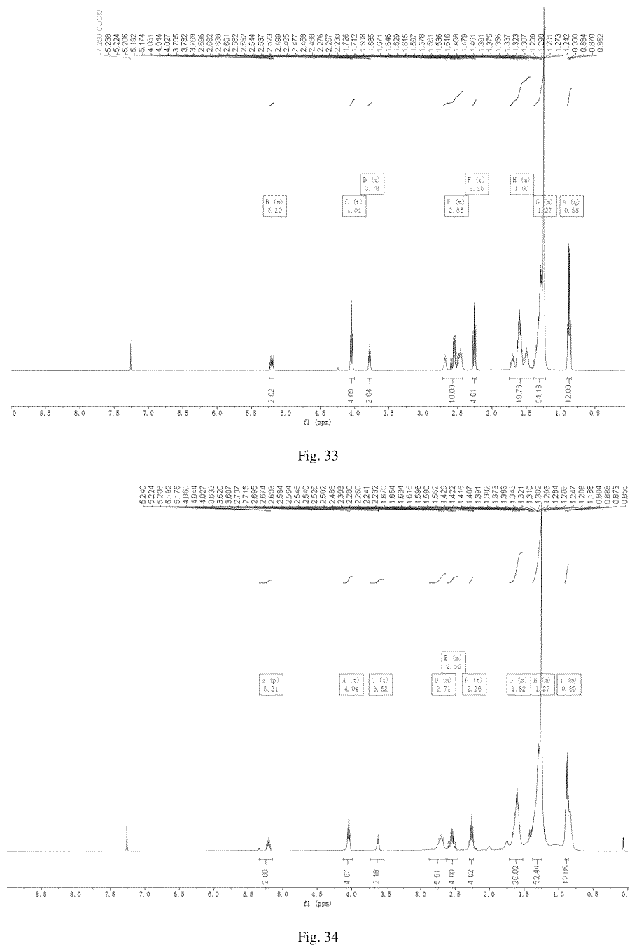

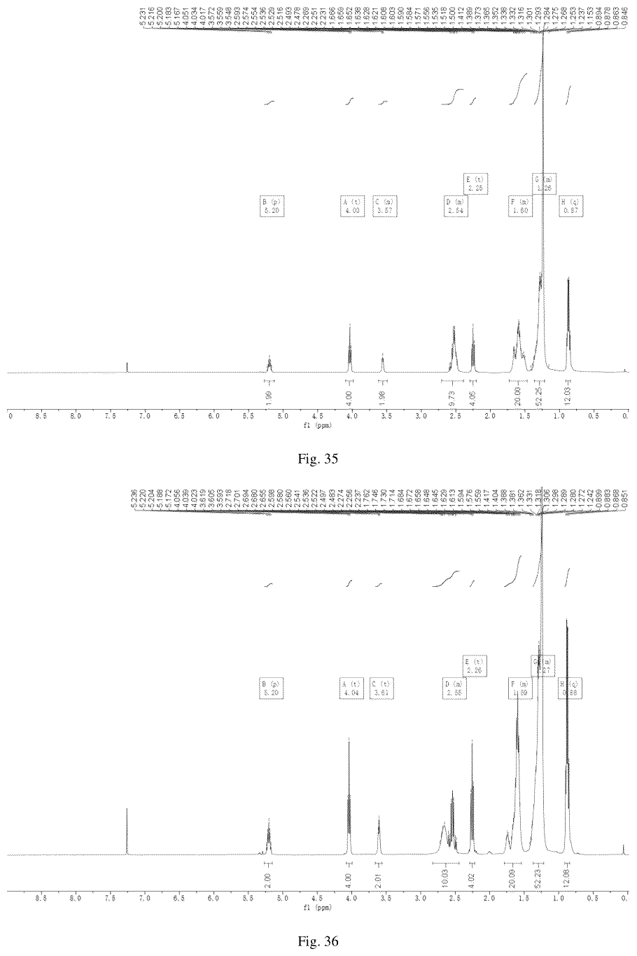

As shown in FIG. 31, in the experimental group of hypodermic injection administration, the effective expression was observed in mice after administration of all formulations. The luciferases expressions of the formulations Rp.14, Rp. 15, Rp. 19, Rp.28 and Rp.29 were superior to other formulations, and were obviously higher than the formulation Rp.58 prepared by the commercial cationic lipid SM-102.

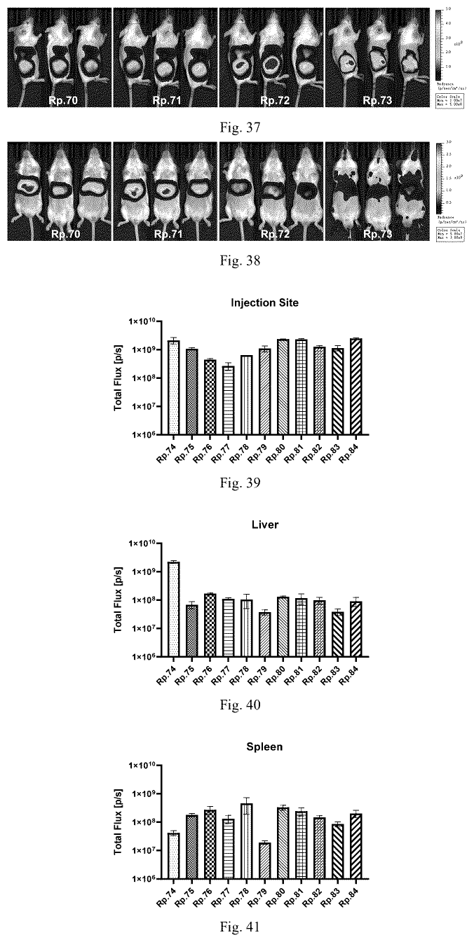

As shown in FIG. 37-38, in the experimental group of intramuscular injection and intravenous administration, the effective expression was observed in mice after administration of Rp.70-Rp.73.

As shown in FIG. 39-46, in the experimental group of intramuscular injection, increasing the proportion of phospholipid in the formulation effectively decreases liver expression, increases spleen expression, and reduces both the ratio of liver/LNs and liver/spleen. Additionally, substituting other components in the formulation does not affect this trend.

As shown in FIG. 47-52, in the experimental group of intravenous injection, increasing the proportion of phospholipid in the formulation effectively decreases liver expression, increases spleen expression, and reduces both the ratio of liver/LNs and liver/spleen. Additionally, substituting other components in the formulation does not affect this trend.

### EXAMPLE 11: EVALUATION OF HUMORAL IMMUNITY EFFECT OF NUCLEIC ACID NANOPARTICLE COMPLEX IN MOUSE

Novel coronavirus S-mRNA was used as an mRNA model, the novel coronavirus S-mRNA is provided by Hongnee Biotech Corporation, and the nucleotide sequence of the novel coronavirus S-mRNA (cap1 structure, N1-me-pseudo U modified) is shown as S-mRNA in the sequence table.

The specific information of the stock solution for S-mRNA:
The product name: COVID-19 Spike Protein, Full Length-mRNA;
Description of the product: the length of 4088 nucleotides;
Modifications: Fully substituted with N1-Me-pseudo UTP; Concentration: 1.0 mg/ml;
Storage environment: 1 mM sodium citrate, pH 6.4;
Storage requirements: −40° C. or below.

### Experimental Process:

Step 1: First immunization of mice: on day 0, 5-6 weeks, female BALB/c mice, 5 mice per group, intramuscular injection was carried out: 125 μL PBS (blank control), 5 μg naked S-mRNA (negative control), 125 μL nucleic acid nanoparticle complex formulations Rp.12, Rp.14, Rp.15, Rp. 17, Rp.19, Rp.30, Rp.38, Rp.58 loaded with 5 μg S-mRNA; intravenous injection injection was carried out: 125 μL of nucleic acid nanoparticle complex injection Rp.03, Rp.09 and Rp.23 loaded with 5 μg S-mRNA; hypodermic injection was carried out: 125 μL of nucleic acid nanoparticle complex formulation Rp. 19 loaded with 5 μg S-mRNA.
Step 2: First serum collection: on day 14, blood was collected via the outer canthus of the mice. After 1 h of blood coagulation at 4° C., the blood was centrifuged for 5 minutes at 4° C. at 5000×g, the supernatant was taken and then centrifuged for 5 minutes at 10000×g at 4° C., the supernatant was taken and added into eight rows of PCR tubes for subpackage and the tubes were frozen and stored for standby at −20° C.
Step 3: Second immunization of mice: on day 14, blood was collected via the outer canthus of the mice and the procedure for the first immunization was repeated.
Step 4: Second serum collection: on day 28, i.e. after 14 days of the second immunization, blood was collected via the outer canthus of the mice. Step 2 was repeated to preserve mouse serum.
Step 5: Serum IgG content was detected using ELISA: the S protein was diluted in PBS and ELISA plates were coated with 100 μL of the dilution (containing 0.5 or 1 μg S protein) per well overnight at 4° C. The liquid in the plate was discarded, the plate was washed for 3 times by addition of 200 μL of PBST into each well, 200 μL of PBS blocking liquid containing 5%-10% BSA was added into each well, and blocked for 2 hours in a shaking table at room temperature. The blocking solution was discarded, the plate was washed for 1 time by addition of 200 μL of PBST into each well, 100 μL of serum diluted 200-fold with PBS was added, and incubated for 2 hours at room temperature on a shaker. The serum was discarded, the plate was washed for 3 times by addition of 200 μL of PBST into each well, 100 μL of antibody dilution (antibody diluted 1:1000 in PBS) was added to each well, and incubated for 1 h at room temperature with shaking. The antibody was discarded, the plate was washed for 3 times by addition of 200 μL of PBST into each well, 50 μL of TMB color development liquid was added into each well, 50 μL of TMB color development solution was added into each well for reaction in dark place, 50 μL of 2M sulfuric acid or 50 μL of 1M phosphoric acid was added into each well after the positive control well turns deep blue or reacting for 10 min to terminate the reaction, the optical density was detected at the wavelength of 450 nm and 630 nm by an enzyme-linked immunosorbent assay, and the OD value difference was calculated to reflect the level of the anti-S protein IgG in the serum.

The results are shown in FIG. 32. Conclusion: the results show that the formulations Rp.03, Rp.09, Rp. 12, Rp. 14, Rp. 15, Rp. 17, Rp. 19, Rp.23, Rp.30, Rp.38 and Rp.58 have OD values obviously higher than those of the blank control group and the naked mRNA negative control group after second immunization, and cause obvious immune response was observed, and the formulations Rp.03, Rp.09, Rp.12, Rp.14, Rp.15, Rp.17, Rp.19, Rp.30 and Rp.38 have the same effect as the formulations Rp.58 of the commercial cationic lipid molecule SM-102, and the formulations of the nucleic acid nanoparticle complexes have stronger serum transformation efficiency and humoral immune activation function.

### EXAMPLE 12: TOXICITY EVALUATION OF NUCLEIC ACID NANOPARTICLE COMPLEXES IN MICE

Sample preparation: nucleic acid nanoparticle complexes comprising FLuc-mRNA were prepared according to the preparation method of the formulation described in example 7. An equal volume of PBS buffer was used as a control.

Test animals: clean grade Balb/c mice, female, 6-8 weeks old, 22-25 g in body weight.

Acute toxicity test in mice: 3 female mice, mRNA formulations (Rp.65, Rp.66, Rp.67, Rp.68, Rp.69) containing various ionizable lipids were prepared according to the method of example 7. The dosage was 50 μg/mouse (based on mRNA content) and an equal volume of PBS buffer was used as a control group, and the administration was tail vein injection. After administration, the mice were observed continuously for 1 week, during which time the physiological condition of the mice, the time of appearance of toxic symptoms, the degree of development of symptoms, the progress of development, the time of death, the characteristics before death, and the number of dead mice and so on were recorded.

Mice in the formulation group Rp.69 containing ALC-0315 died sequentially after 24 h of administration. No obvious toxic reaction was observed in the Rp.65, Rp.66, Rp.67 and Rp.68 groups containing the compound provided herein and the PBS (phosphate buffer solution) administration group in one week, this indicates the safety of the compound provided herein.

While the methods of the present invention have been described in terms of preferred embodiments, it will be apparent to those of ordinary skill in the art that variations and modifications of the methods and applications described herein, as well as other suitable variations and combinations, may be made to implement and use the techniques of the present invention within the intended scope, spirit and scope of the invention. Those skilled in the art can modify the process parameters appropriately in view of the disclosure herein. It is specifically noted that all such substitutions and modifications will be apparent to those skilled in the art and are intended to be included within the present invention.

### EMBODIMENTS

1. A compound of formula A,

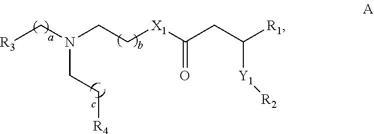

wherein a is an integer from 0 to 10;
b is an integer from 1 to 9;
c is an integer from 1 to 9;
R1 is C1-C20 alkyl;
R2 is hydrogen or —C(═O)Ra;
Ra is C1-C20 alkyl;
R3 is hydroxy,

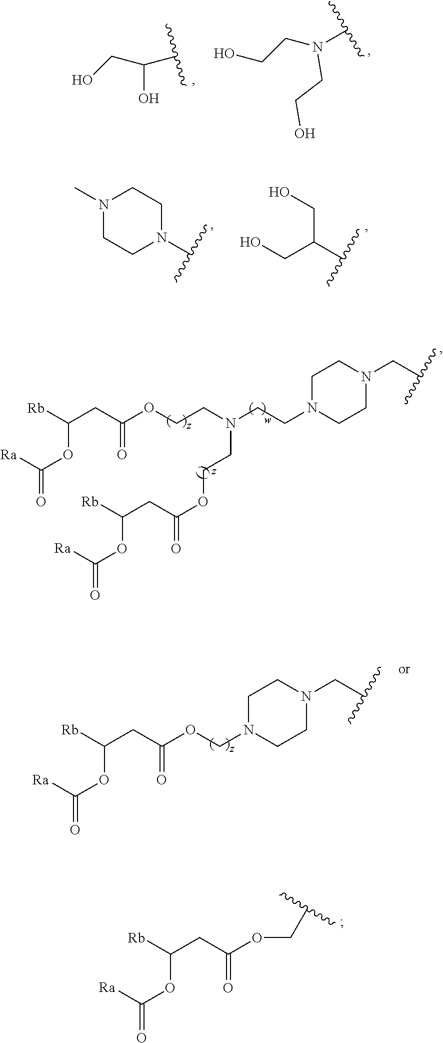

R4 is

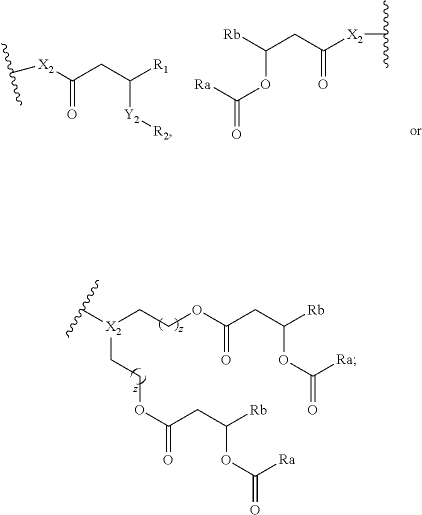

Rb is C1-C20 alkyl;
w is an integer from 1 to 9,
z is an integer from 1 to 9;
X1 is O or NH;
each X2 is independently O or NH;
Y1 is O or NH;
Y2 is O or NH.
2. The compound of embodiment 1, wherein the compound of formula A is selected from a compound of formula I, a compound of formula II, a compound of formula III and a compound of formula IV,

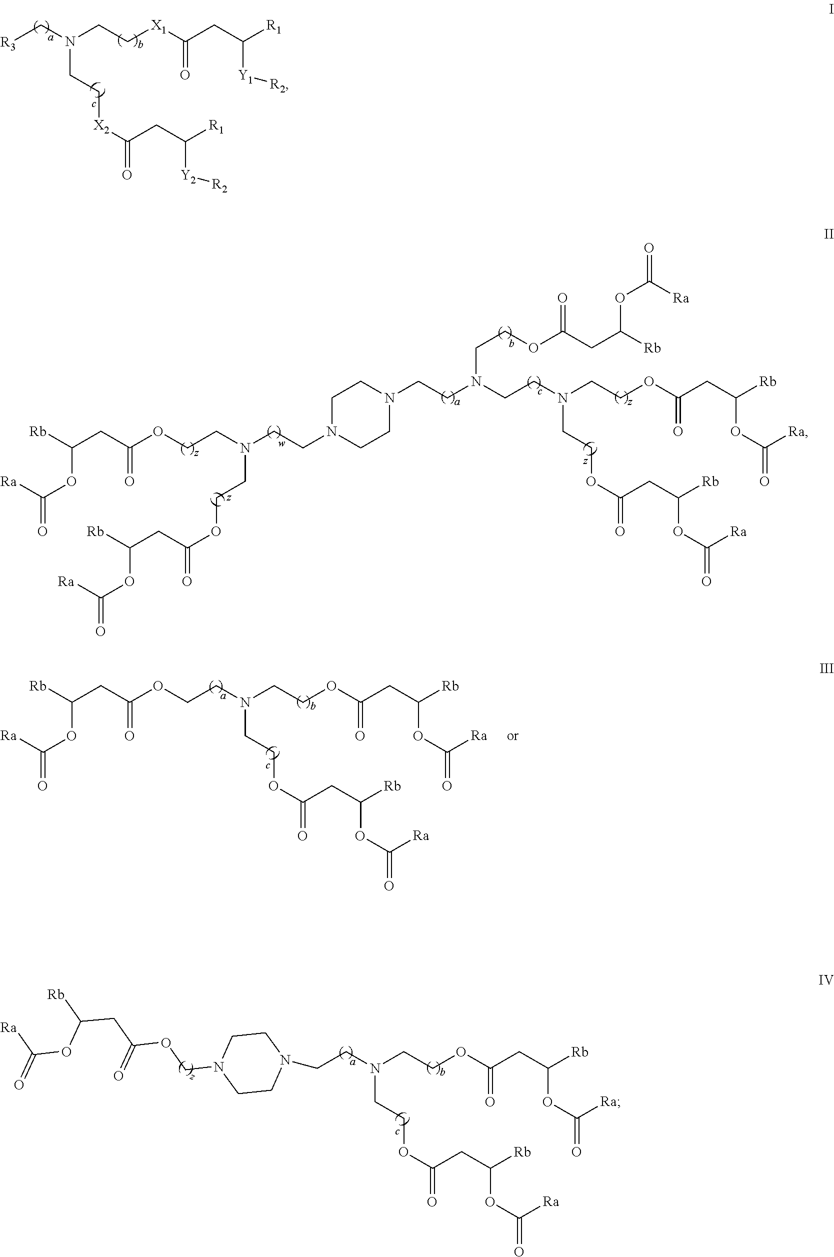

wherein in the compound of formula II, w is an integer from 1 to 9; z is an integer from 1 to 9;
in the compound of formula I, R3 is hydroxy,

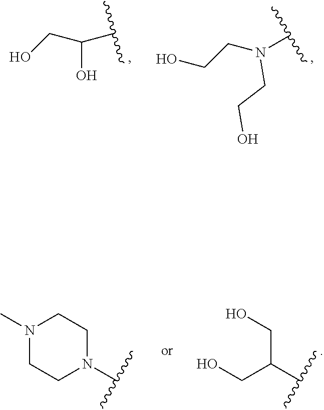

3. The compound of embodiment 2, wherein the compound of formula A is selected from a compound of formula I, wherein R1 is unsubstituted C1-C15 straight chain alkyl, R2 is hydrogen or —C(═O)Ra, Ra is unsubstituted C5-C15 straight chain alkyl, a is an integer from 0 to 6, b is an integer from 3 to 5, c is an integer from 3 to 5; or
the compound of formula A is selected from a compound of formula I, wherein R1 is unsubstituted C5-C11 straight chain alkyl, R2 is hydrogen or —C(═O)Ra, Ra is unsubstituted C5-C13 straight chain alkyl, a is an integer from 0 to 6, b is an integer from 3 to 5, c is an integer from 3 to 5.
4. The compound of embodiment 3, wherein the compound of formula A is selected from a compound of formula I, the compound of formula I is selected from compound L0111, compound L0112, compound L0113, compound L0114, compound L0115, compound L0116, compound L0117, compound L0118, compound L0119, compound L0120, compound L0121, compound L0122, compound L0123, compound L0124, compound L0125, compound L0132, compound L0133, compound L0134, compound L0135, compound L0136, compound L0137, compound L0138, compound L0139 and compound L0140,

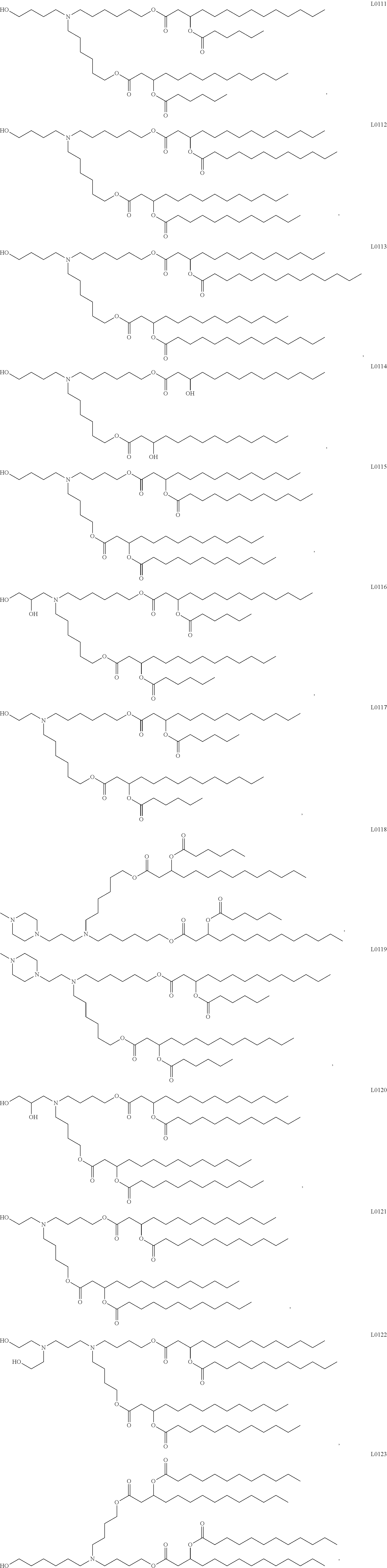

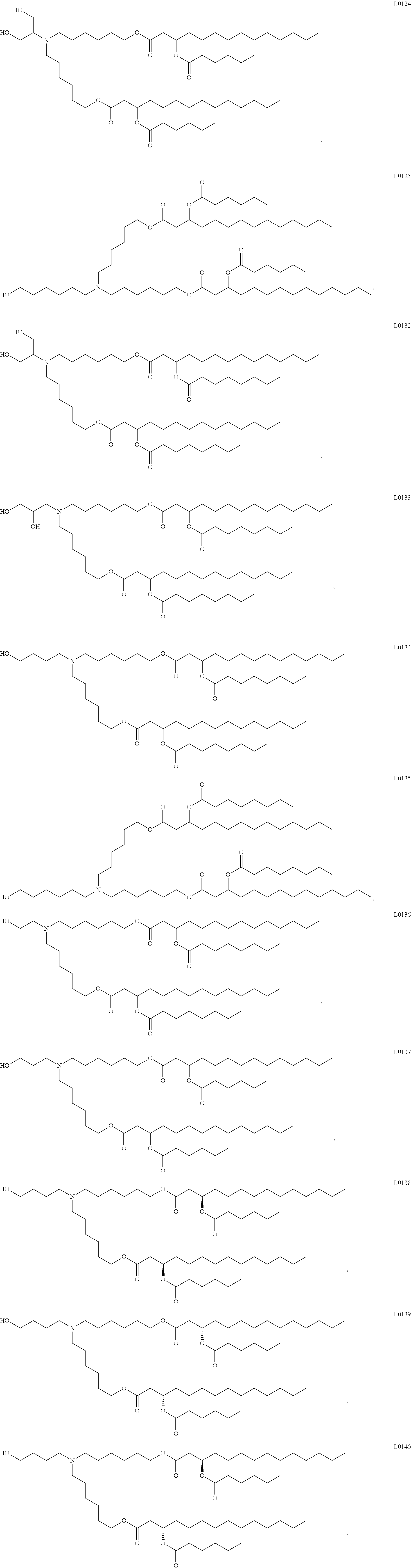

5. The compound of embodiment 2, wherein the compound of formula A is selected from a compound of formula II, wherein a is an integer from 0 to 5, b is an integer from 3 to 10, c is an integer from 1 to 5, w is an integer from 1 to 5, z is an integer from 5 to 10, Ra is unsubstituted C5-C15 straight chain alkyl, Rb is unsubstituted C5-C15 straight chain alkyl; or
the compound of formula A is selected from a compound of formula II, wherein a is an integer from 0 to 3, b is an integer from 4 to 6, c is an integer from 1 to 3, w is an integer from 1 to 3, z is an integer from 5 to 8, Ra is unsubstituted C5-C13 straight chain alkyl, Rb is unsubstituted C9-C13 straight chain alkyl; or
the compound of formula A is selected from a compound of formula II, wherein a is 1, b is 5, c is 1, w is 1, z is 6, Ra is unsubstituted C5-C13 straight chain alkyl, Rb is unsubstituted C10 straight chain alkyl, unsubstituted C11 straight chain alkyl or unsubstituted C12 straight chain alkyl.
6. The compound of embodiment 5, wherein the compound of formula A is selected from a compound of formula II, the compound of formula II is selected from compound L0126, compound L0127 and compound L0128,

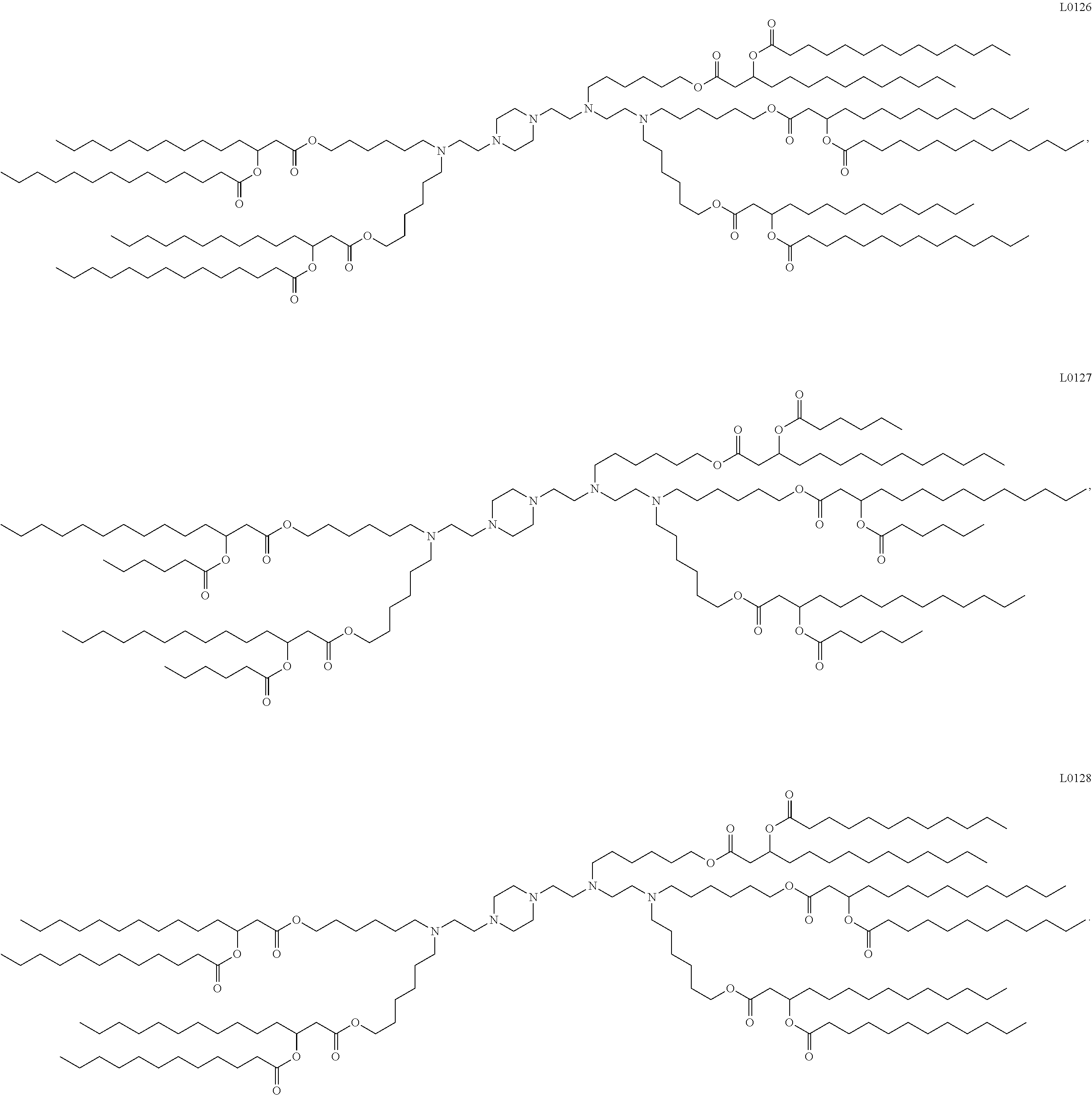

7. The compound of embodiment 2, wherein the compound of formula A is selected from a compound of formula III, wherein a, b and c are the same and are an integer from 1 to 9; or
the compound of formula A is selected from a compound of formula III, wherein a, b and c are the same and are an integer from 1 to 5; or
the compound of formula A is selected from a compound of formula III, wherein a, b and c are the same and are an integer from 1 to 5; Ra is unsubstituted C5-C15 straight chain alkyl, Rb is unsubstituted C5-C15 straight chain alkyl; or
the compound of formula A is selected from a compound of formula III, wherein a, b and c are the same and are an integer from 3 to 5; Ra is unsubstituted C5-C11 straight chain alkyl, Rb is unsubstituted C8-C13 straight chain alkyl; or
the compound of formula A is selected from a compound of formula III, wherein a, b and c are the same and are an integer from 3 to 5; Ra is unsubstituted C5-C11 straight chain alkyl, Rb is unsubstituted C11 straight chain alkyl.
8. The compound of embodiment 7, wherein the compound of formula A is selected from a compound of formula III, the compound of formula III is selected from compound L0129 or compound L0130,

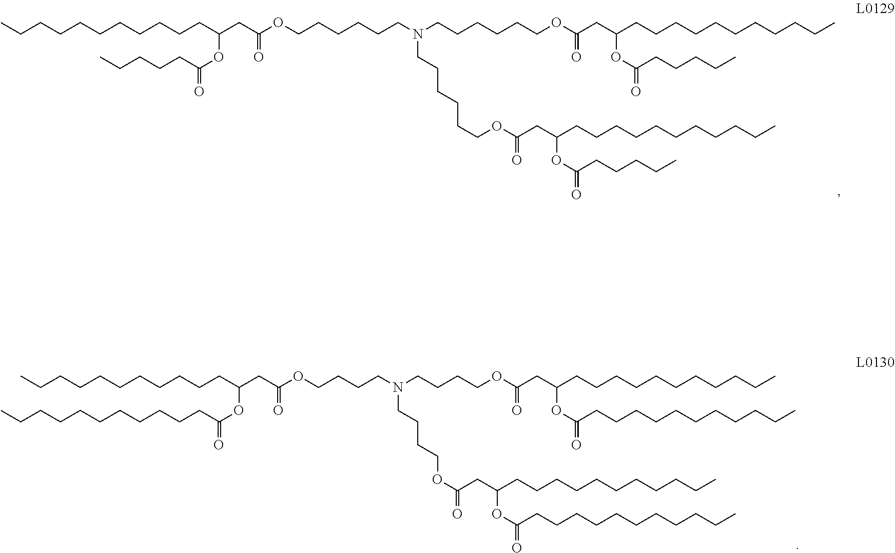

9. The compound of embodiment 2, wherein the compound of formula A is selected from a compound of formula IV, wherein a is an integer from 0 to 5, b is an integer from 3 to 10, c is an integer from 3 to 10, z is an integer from 5 to 10, Ra is unsubstituted C5-C15 straight chain alkyl, Rb is unsubstituted C5-C15 straight chain alkyl; or
the compound of formula A is selected from a compound of formula IV, wherein a is an integer from 0 to 3, b is an integer from 4 to 6, c is an integer from 4 to 6, z is an integer from 5 to 8, Ra is unsubstituted C5-C13 straight chain alkyl, Rb is unsubstituted C9-C13 straight chain alkyl; or
the compound of formula A is selected from a compound of formula IV, wherein a is 1, b is 5, c is 5, z is 6, Ra is unsubstituted C5-C13 straight chain alkyl, Rb is unsubstituted C10-C15 straight chain alkyl.
10. The compound of embodiment 2 or 9, wherein the compound of formula A is selected from a compound of IV, the compound of formula IV is selected from compound L0129 and compound L0131,

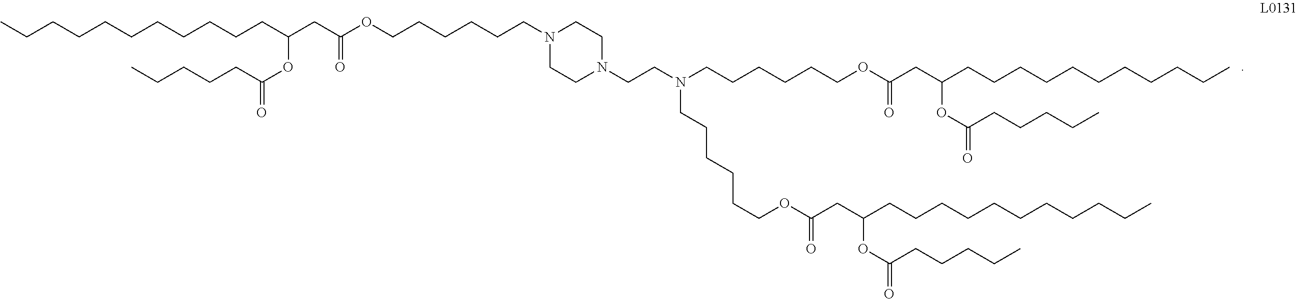 [SMILES: CCCCCCCCCCCC(CC(=O)OCCCCCCN(CCCCCCOC(=O)CC(CCCCCCCCCCC)OC(=O)CCCCC)CCN1CCN(CCCCCCOC(=O)CC(CCCCCCCCCCC)OC(=O)CCCCC)CC1)OC(=O)CCCCC]

11. A lipid compound nanoparticle comprising the following components: a compound of any one of embodiments 1 to 10 and auxiliary materials;
optionally, the auxiliary material includes at least one of a PEG derivative, a lipid, a lipid-like substance, an alcohol, a saccharide and an inorganic salt.
12. The lipid compound nanoparticle of embodiment 11, wherein the PEG derivative includes at least one of PEG modified phosphatidylethanolamine, PEG modified phosphatidic acid, PEG modified ceramide, PEG modified dialkylamine, PEG modified diacylglycerol, PEG modified dialkyglycerol, PEG modified stearic acid and PEG modified phosphatidylserine; and/or
the PEG derivative includes at least one of 1,2-dimyristoyl-sn-glycero methoxypolyethylene glycol, 1,2-distearoyl-sn-glycero-3-phosphoethanolamine-N-(amino(polyethylene glycol)), dilauroyl phosphatidylethanolamine-polyethylene glycol, dimyristoyl phosphatidylethanolamine -polyethylene glycol, dipalmitoyl phosphatidylcholine polyethylene glycol, dipalmitoyl phosphatidylethanolamine-polyethylene glycol, PEG-distearoyl glycerol, PEG-dipalmitoyl, PEG-dioleoyl, PEG-distearoyl, PEG-diacylglycerol amide, PEG-dipalmitoyl phosphatidylethanolamine and PEG-1,2-dimyristol oxypropyl-3-amine; and/or
the PEG derivative includes at least one of PEG-c-DOMG, PEG-c-DMA, PEG-DMG, PEG-DSG, PEG-DAG, PEG-DLPE, PEG-DPPC, ALC-0159, PEG-Mal, DSPE-PEG-Mal, DMG-PEG2000, mPEG-DSPE, mPEG-STA, mPEG-PS, mPEG-DMPE and mPEG-DPPE; and/or
the lipid includes at least one of phospholipid and sterol; and/or
the phospholipid includes at least one of lecithin, 1,2-distearoyl-sn-glycero-3-phosphocholine, 1,2-dioleoyl-sn-glycero-3-phosphoethanolamine, 1,2-dioleoyl-sn-glycero-3-phosphocholine, 1,2-dimyristoyl-sn-glycero-3-phosphocholine, 1,2-dioleoyl-sn-glycero-3-phosphocholine, 1,2-dipalmitoyl-sn-glycero-3-phosphocholine, 1,2-diundecanoyl-sn-glycero-phosphocholine, 1-palmitoyl-2-oleoyl-sn-glycero-3-phosphocholine, Dilauroylphosphatidylcholine (DLPC), 1,2-Diheneicosanoyl-sn-glycero-3-phosphocholine (DUPC), 1-Palmitoyl-2-oleoyl-sn-glycero-3-phosphocholine (POPC), 1,2-Diisooctyl-OT-glycero-3-phosphocholine (18:0 Diether PC), 1-Oleoyl-2-cholesterylhemisuccinoyl-OT-glycero-S-phosphocholine (OChemSPC), 1,2-Dilinolenoyl-sn-glycero-3-phosphocholine, 1,2-Diarachidonoyl-sn-glycero-3-phosphocholine, 1,2-Didocosahexaenoyl-sn-glycero-3-phosphocholine, 1,2-Diphenylglyceryl-sn-glycero-3-phosphoethanolamine (ME16.0 PE), 1,2-Distearoyl-sn-glycero-3-phosphoethanolamine, 1,2-Dioctanoyl-sn-glycero-3-phosphoethanolamine, 1,2-Dilinoleoyl-sn-glycero-3-phosphoethanolamine, 1,2-Diarachidonoyl-sn-glycero-3-phosphoethanolamine, 1,2-Didocosahexaenoyl-sn-glycero-3-phosphoethanolamine, 1,2-Dioleoyl-sn-glycero-3-phospho-L -glycerol Sodium Salt (DOPG), Monopalmitoyl Phosphatidylcholine (MPPC), Myristoyl Stearoyl Phosphatidylcholine (MSPC), 1-palmitoyl-2-myristoyl-sn-glycero-3-phosphocholine (PMPC), 1-Palmitoyl-2-stearoyl-sn-glycero-3-phosphocholine (PSPC), 1-stearoyl-2-myristoyl-sn-glycero-3-phosphocholine (SMPC), 1-Stearoyl-2-palmitoyl-sn-glycero-3-phosphocholine (SPPC), 2-Oleoyl-1-stearoyl-sn-glycero-3-phosphocholine (SOPC), 1,2-Ditetradecyl-rac-glycero-3-phosphoethanolamine (DMPE), 1,2-Distearoyl-sn-glycero-3-phosphorylethanolamine (DSPE), and Sphingomyelin; and/or
the sterol includes at least one of cholesterol, lanosterol, 5 α-cholestan-3 β-alcohol, coprosterol, sitosterol, ergosterol, campesterol, stigmasterol, brassicasterol, tomatidine, ursolic acid and α-tocopherol.
13. The lipid compound nanoparticle of any one of embodiments 11 to 12, wherein the lipid compound nanoparticle includes the compound of any one of embodiments 1 to 10, a PEG derivative and a lipid; the lipid includes at least one of phospholipid and sterol; and/or
calculated on the total molar amount of each component of the lipid compound nanoparticle, the compound of any one of embodiments 1 to 10 has an amount of 14.8 mol %-70.0 mol %; and/or
calculated on the total molar amount of each component of the lipid compound nanoparticle, the PEG derivative has an amount of 0.4 mol %-10 mol %; and/or
calculated on the total molar amount of each component of the lipid compound nanoparticle, the phospholipid has an amount of 5.0 mol %-50.0 mol %; and/or
calculated on the total molar amount of each component of the lipid compound nanoparticle, the sterol has an amount of 10.0 mol %-75.0 mol % or 15.0mol %-75.0 mol%.
14. The lipid compound nanoparticle of any one of embodiments 11 to 13, wherein a molar ratio of PEG derivative:phospholipid:sterol: the compound of any one of embodiments 1 to 10 is (0.4-10.0):(5.0-50.0):(10.0-75.0):(14.8-70.0) or (0.4-10.0):(5.0-40.0):(15.0-75.0):(14.8-70.0): or
a molar ratio of PEG derivative:phospholipid:sterol: the compound of any one of embodiments 1 to 10 is 2.50:16.00:16.50:65.00, 1.00:5.00:64.00:30.00, 0.40:8.00:56.60:35.00, 1.00:8.00:61.00:30.00, 1.20:12.00:38.30:48.50, 1.70:9.00:40.80:48.50, 2.50:16.00:21.50:60.00, 1.20:9.00:47.80:42.00, 1.00:16.00:34.50:48.50, 2.50:8.00:41.00:48.50, 1.50:8.00:25.50:65.00, 2.50:11.50:51.00:35.00, 1.50:8.00:30.50:60.00, 1.00:5.00:62.00:32.00, 0.60:5.00:54.40:40.00, 1.50:16.00:47.50:35.00, 1.63:12.99:45.38:40.00, 1.20:8.00:55.8:35.00, 1.30:8.00:55.70:35.00, 0.40:5.00:59.60:35.00, 1.00:20.00:37.00:42.00, 1.00:25.00:34.00:40.00, 3.20:12.00:29.80:55.00, 1.00:8.00:53.00:38.00, 2.50:9.50:33.00:55.00, 10.00:16.00:54.00:20.00, 3.00:30.00:42.00:25.00, 3.20:16.80:10.00:70.00, 3.00:17.00:25.00:55.00, 3.20:16.80:15.00:65.00, 4.20:11.00:70.00:14.80, 6.20:6.80:75.00:15.00, 1.50:11.50:38.50:48.50, 7.50:9.61:35.56:47.33, 3.00:9.50:32.50:55.00, 0.95:7.58:26.47:65.00, 1.40:11.15:38.95:48.50, 2.10:7.98:47.90:42.02, 3.00:6.00:43.00:48.00, 1.60:30.00:30.00:40.00, 1.60:35.00:35.00:30.00, 1.60:40.00:40.00:20.00, or 1.80:40.00:27.20:31.00; or
a molar ratio of PEG derivative:phospholipid:sterol: the compound of any one of embodiments 1 to 10 is (0.4-1.5):(8.0-11.5):(38.5-56.6):(35.0-48.5): or
a molar ratio of PEG derivative:phospholipid:sterol: the compound of any one of embodiments 1 to 10 is 0.40:8.00:56.60:35.00, 1.50:11.50:38.50:48.50 or 1.40:11.15:38.95:48.50.
15. A nucleic acid nanoparticle complex, comprising a nucleic acid and at least one lipid compound nanoparticle of any one of embodiments 11 to 13.
16. The nucleic acid nanoparticle complex of embodiment 15, wherein a molar ratio of ionizable nitrogen atoms in the compound of any one of embodiments 1 to 10 to phosphorus atoms of the nucleic acid in the nucleic acid nanoparticle complex is 5-50.
17. A pharmaceutical composition, comprising the lipid compound nanoparticle of any one of embodiments 11 to 14 or the nucleic acid nanoparticle complex of any one of embodiments 15 to 16, and a pharmaceutically acceptable adjuvant.
18. Use of the compound of any one of embodiments 1 to 10, the lipid compound nanoparticle of any one of embodiments 11 to 14, the nucleic acid nanoparticle complex of any one of embodiments 15 to 16 or the pharmaceutical composition of embodiment 17 in the manufacture of a product for delivering nucleic acids in vivo.
19. A method of delivering nucleic acids in vivo, comprising administering to a subject in need thereof the compound of any one of embodiments 1 to 10, the lipid compound nanoparticle of any one of embodiments 11 to 14, the nucleic acid nanoparticle complex of any one of embodiments 15 to 16, or the pharmaceutical composition of embodiment 17.

What is claimed is:

1. A compound of formula A, 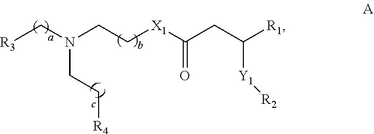
wherein a is an integer from 0 to 10;
b is an integer from 1 to 9;
c is an integer from 1 to 9;
R1 is C1-C20 alkyl;
R2 is hydrogen or —C(═O)Ra;
Ra is C1-C20 alkyl;
R3 is hydroxy, 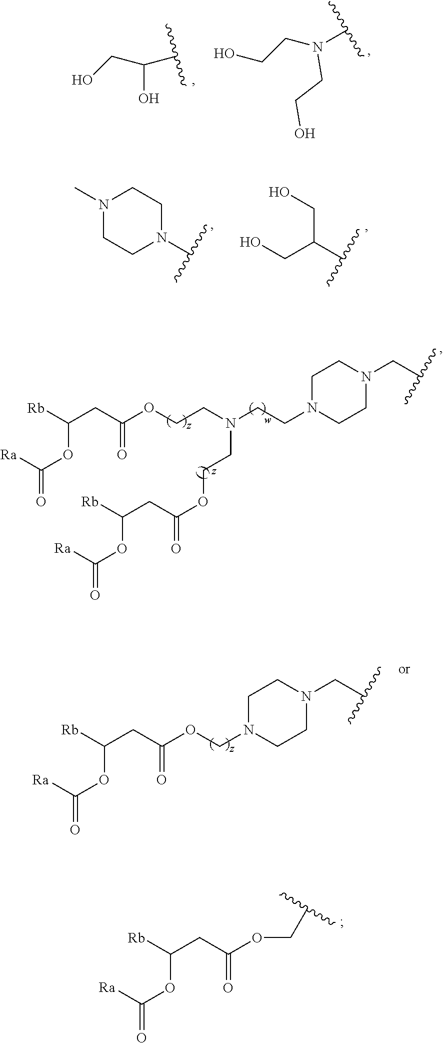
R4 is 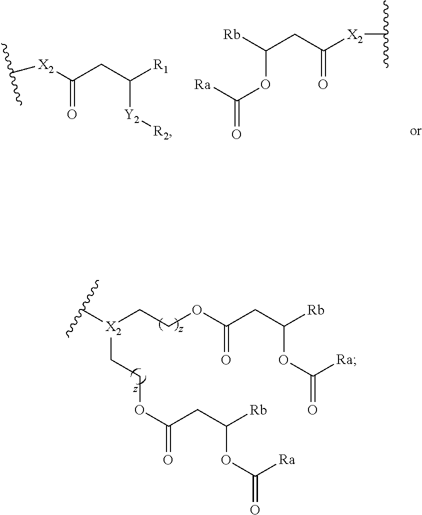
Rb is C1-C20 alkyl;
w is an integer from 1 to 9;
z is an integer from 1 to 9;
X1 is O or NH;
each X2 is independently O or NH;
Y1 is O or NH;
Y2 is O or NH.

2. The compound of claim 1, wherein the compound of formula A is selected from a compound of formula I, a compound of formula II, a compound of formula III and a compound of formula IV, 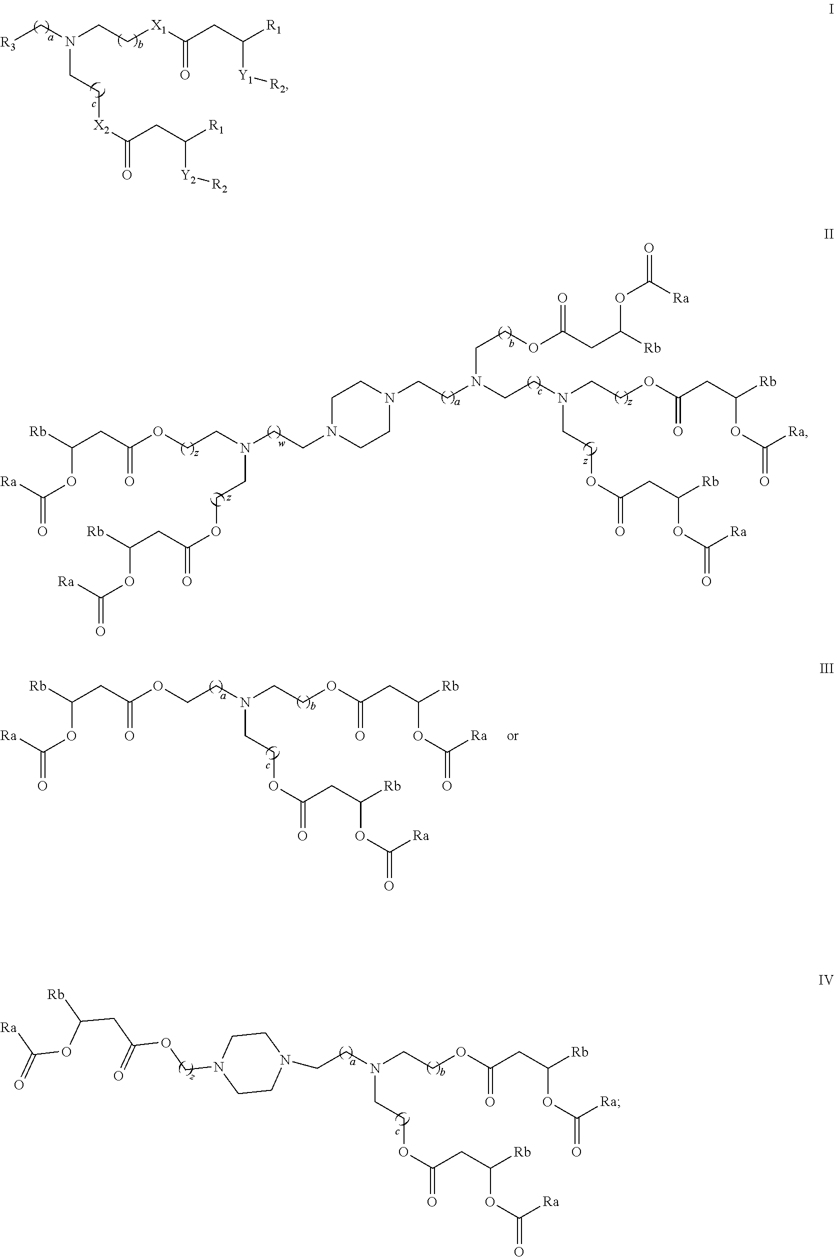
wherein in the compound of formula II, w is an integer from 1 to 9); z is an integer from 1 to 9;
in the compound of formula I, R3 is hydroxy,

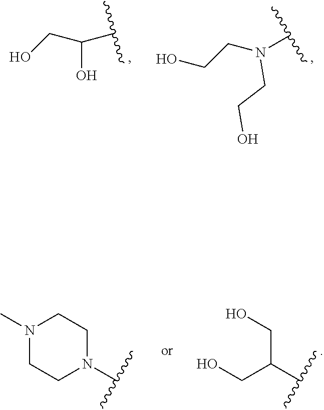

3. The compound of claim 2, wherein the compound of formula A is selected from a compound of formula I, wherein R1 is unsubstituted C1-C15 straight chain alkyl, R2 is hydrogen or —C(═O)Ra, Ra is unsubstituted C5-C15 straight chain alkyl, a is an integer from 0 to 6, b is an integer from 3 to 5, c is an integer from 3 to 5; or
the compound of formula A is selected from a compound of formula I, wherein R1 is unsubstituted C5-C11 straight chain alkyl, R2 is hydrogen or —C(═O)Ra, Ra is unsubstituted C5-C13 straight chain alkyl, a is an integer from 0 to 6, b is an integer from 3 to 5, c is an integer from 3 to 5.

4. The compound of claim 3, wherein the compound of formula A is selected from a compound of formula I, the compound of formula I is selected from compound L0111, compound L0112, compound L0113, compound L0114, compound L0115, compound L0116, compound L0117, compound L0118, compound L0119, compound L0120, compound L0121, compound L0122, compound L0123, compound L0124, compound L0125, compound L0132, compound L0133, compound L0134, compound L0135, compound L0136, compound L0137, compound L0138, compound L0139 and compound L0140,

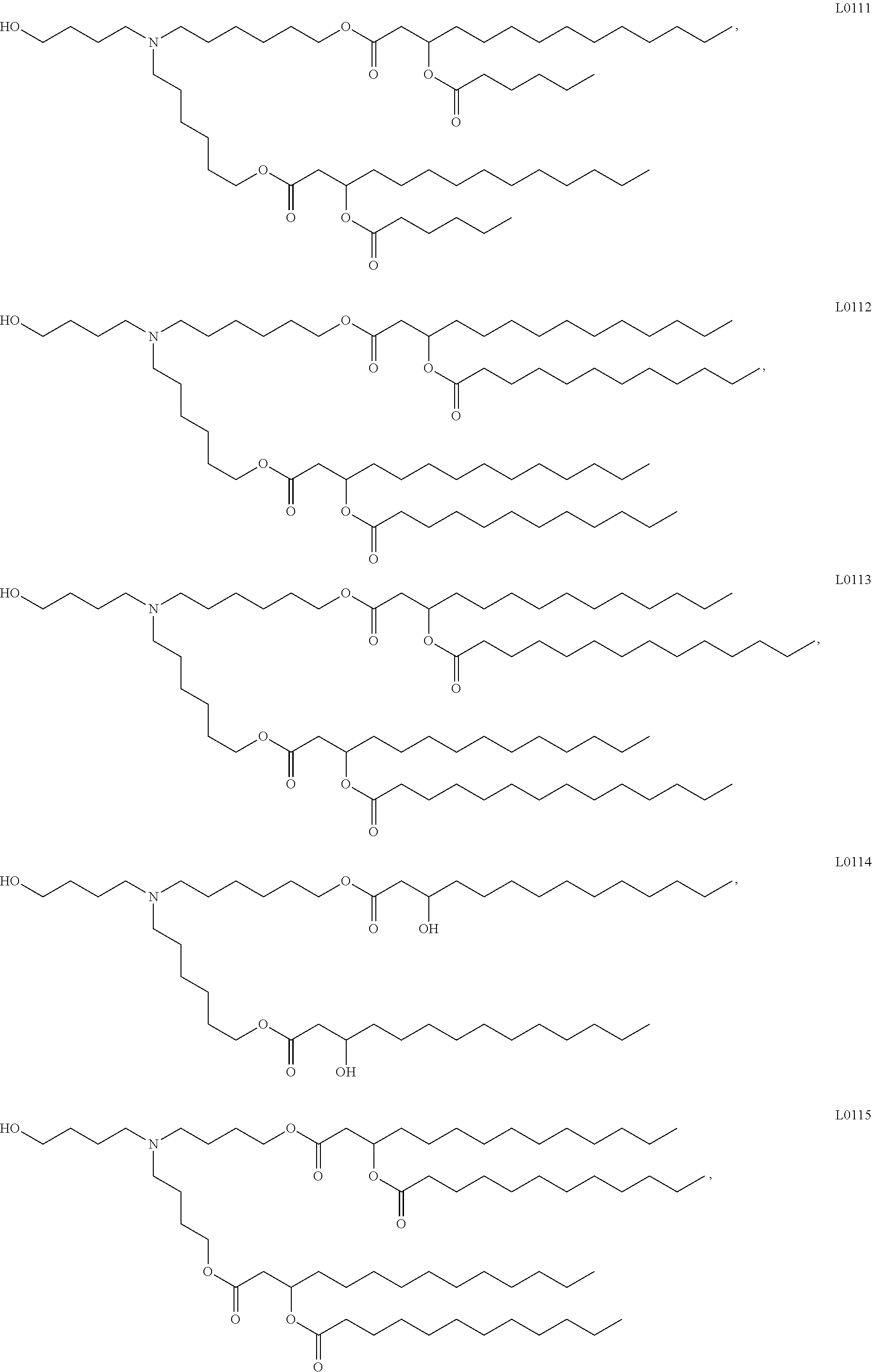

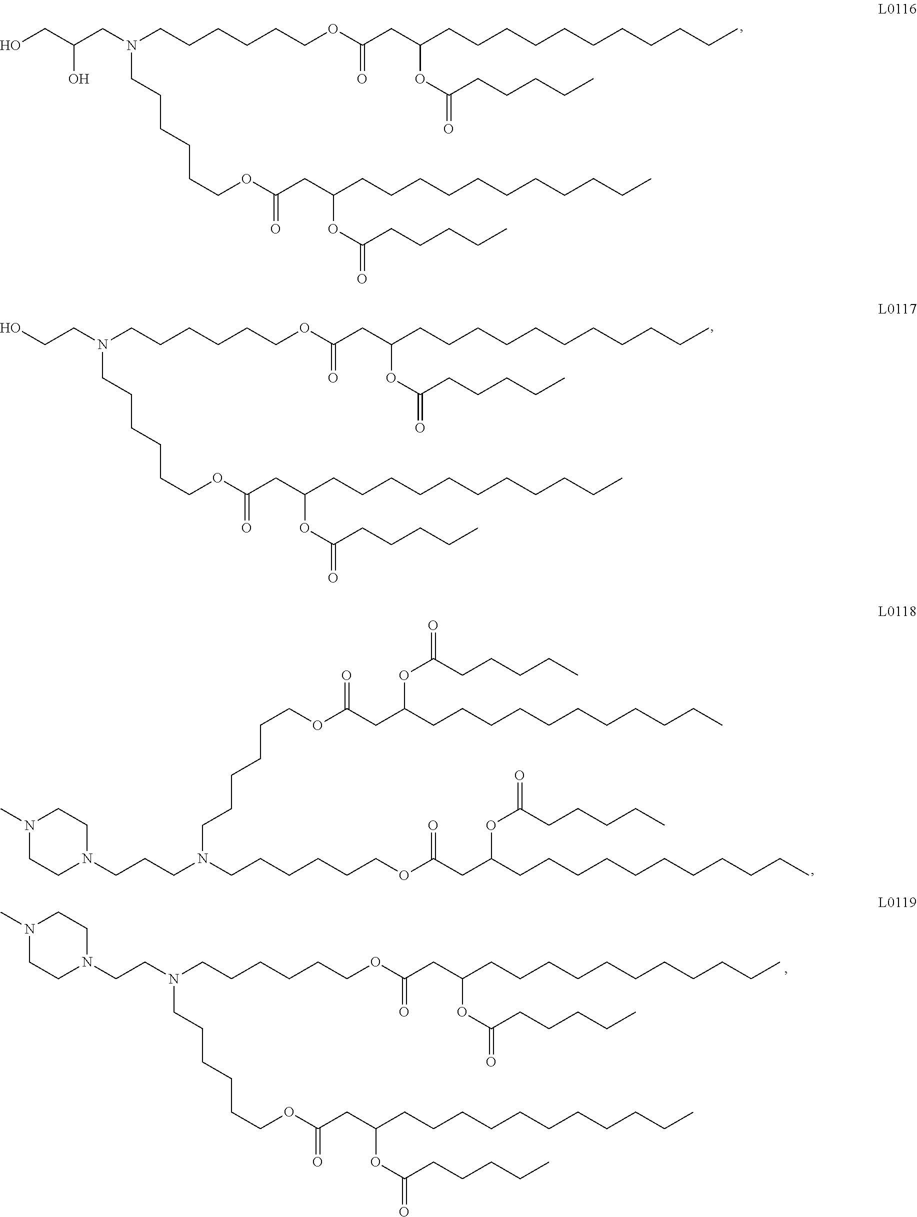

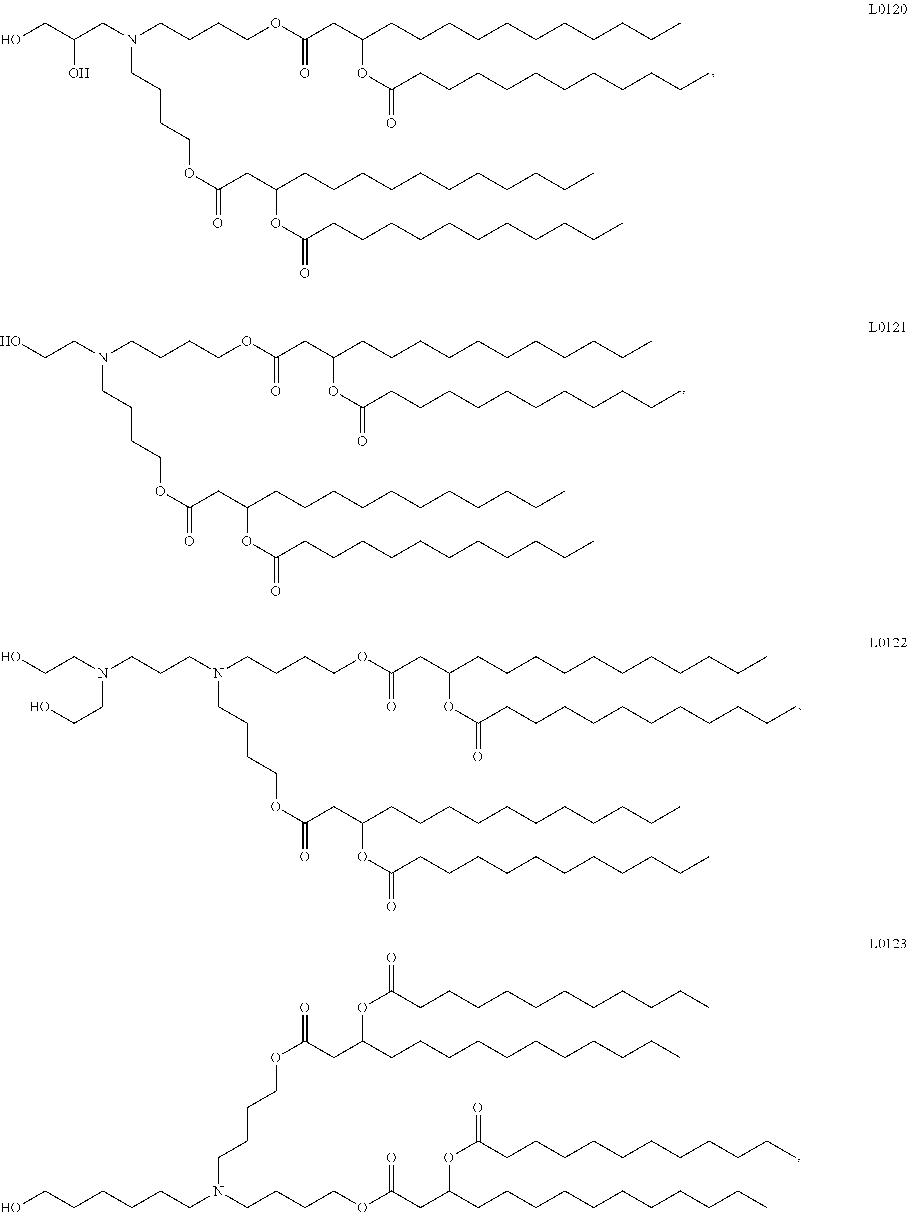 [SMILES: CCCCCCCCCCCC(=O)OC(CCCCCCCCCCC)CC(=O)OCCCCN(CCCCOC(=O)CC(CCCCCCCCCCC)OC(=O)CCCCCCCCCCC)CC(O)CO ; CCCCCCCCCCCC(=O)OC(CCCCCCCCCCC)CC(=O)OCCCCN(CCO)CCCCOC(=O)CC(CCCCCCCCCCC)OC(=O)CCCCCCCCCCC ; CCCCCCCCCCCC(=O)OC(CCCCCCCCCCC)CC(=O)OCCCCN(CCCCOC(=O)CC(CCCCCCCCCCC)OC(=O)CCCCCCCCCCC)CCCN(CCO)CCO ; CCCCCCCCCCCC(=O)OC(CCCCCCCCCCC)CC(=O)OCCCCN(CCCCCCO)CCCCOC(=O)CC(CCCCCCCCCCC)OC(=O)CCCCCCCCCCC]

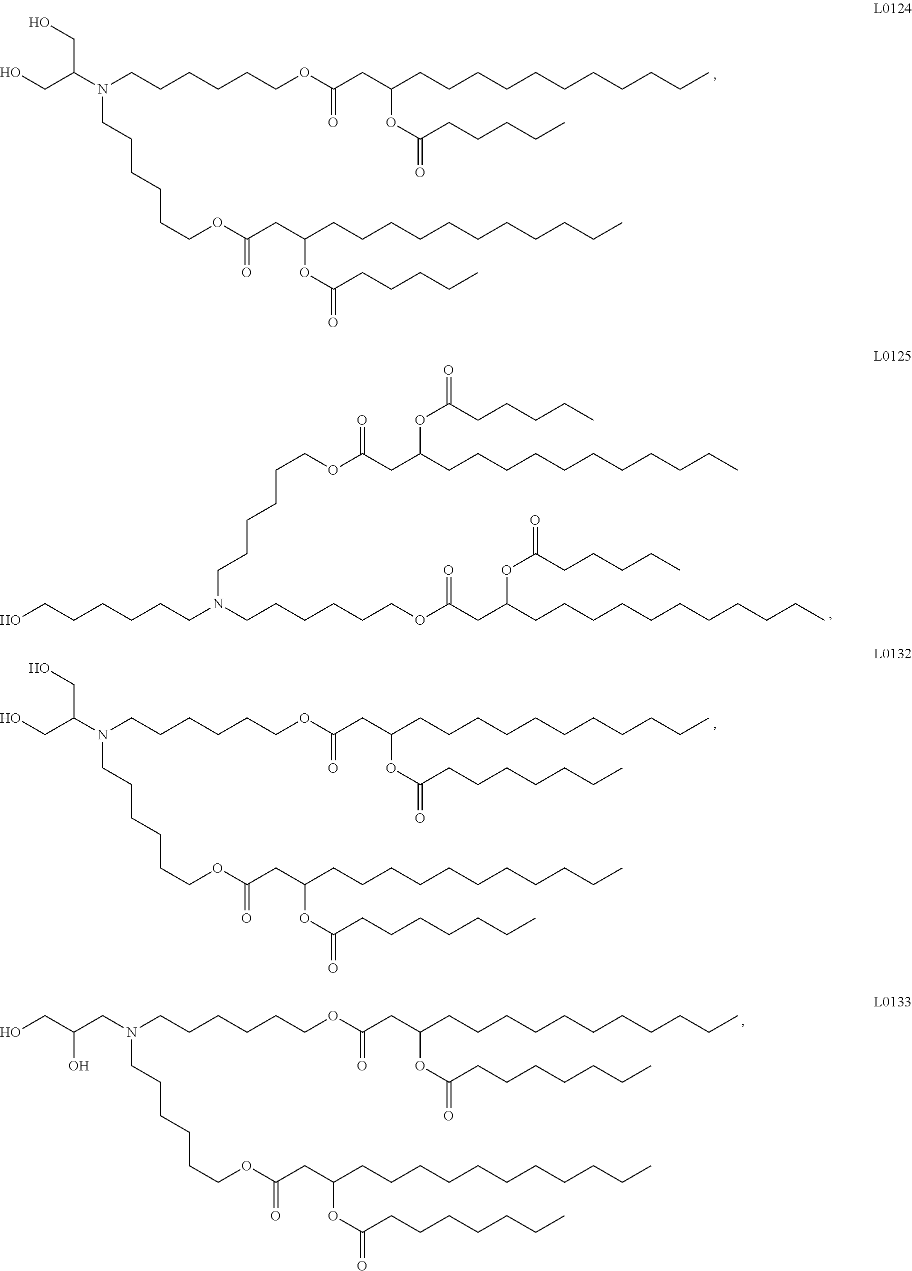 [SMILES: CCCCCCCCCCCC(CC(=O)OCCCCCCN(CCCCCCOC(=O)CC(CCCCCCCCCCC)OC(=O)CCCCC)C(CO)CO)OC(=O)CCCCC ; CCCCCCCCCCCC(CC(=O)OCCCCCCN(CCCCCCO)CCCCCCOC(=O)CC(CCCCCCCCCCC)OC(=O)CCCCC)OC(=O)CCCCC ; CCCCCCCCCCCC(CC(=O)OCCCCCCN(CCCCCCOC(=O)CC(CCCCCCCCCCC)OC(=O)CCCCCCC)C(CO)CO)OC(=O)CCCCCCC ; CCCCCCCCCCCC(CC(=O)OCCCCCCN(CCCCCCOC(=O)CC(CCCCCCCCCCC)OC(=O)CCCCCCC)CC(O)CO)OC(=O)CCCCCCC]

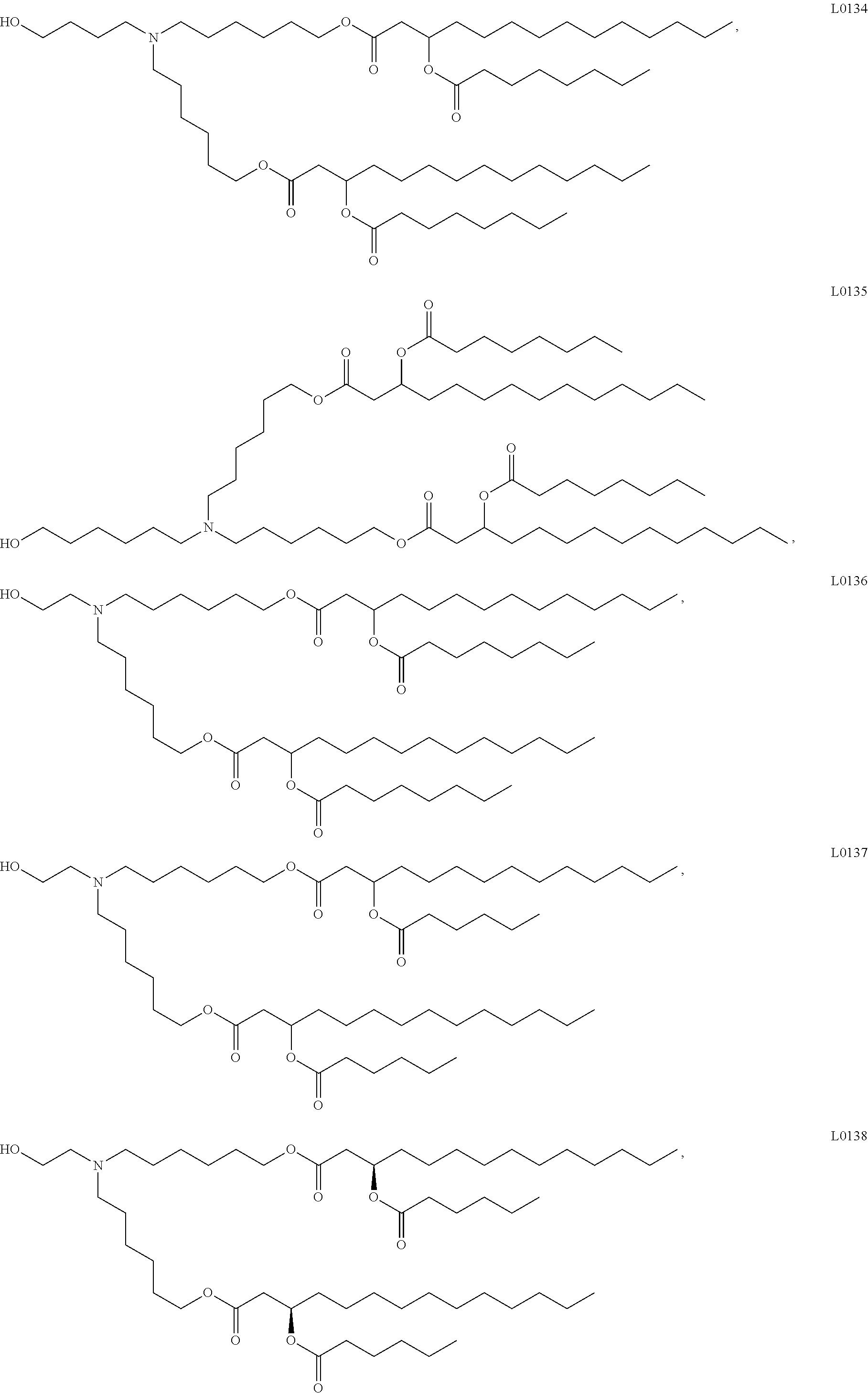

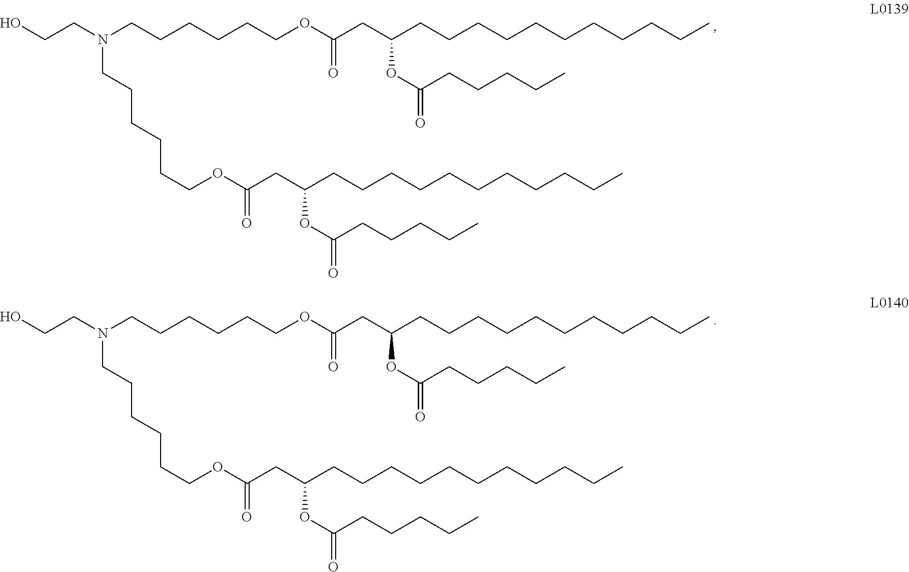 [SMILES: CCCCCCCCCCC[C@@H](CC(=O)OCCCCCCN(CCO)CCCCCCOC(=O)C[C@H](CCCCCCCCCCC)OC(=O)CCCCC)OC(=O)CCCCC ; CCCCCCCCCCC[C@H](CC(=O)OCCCCCCN(CCO)CCCCCCOC(=O)C[C@H](CCCCCCCCCCC)OC(=O)CCCCC)OC(=O)CCCCC]

5. The compound of claim 2, wherein the compound of formula A is selected from a compound of formula II, wherein a is an integer from 0 to 5, b is an integer from 3 to 10, c is an integer from 1 to 5, w is an integer from 1 to 5, z is an integer from 5 to 10, Ra is unsubstituted C5-C15 straight chain alkyl, Rb is unsubstituted C5-C15 straight chain alkyl; or
the compound of formula A is selected from a compound of formula II, wherein a is an integer from 0 to 3, b is an integer from 4 to 6, c is an integer from 1 to 3, w is an integer from 1 to 3, z is an integer from 5 to 8, Ra is unsubstituted C5-C13 straight chain alkyl, Rb is unsubstituted C9-C13 straight chain alkyl; or
the compound of formula A is selected from a compound of formula II, wherein a is 1, b is 5, c is 1, w is 1, z is 6, Ra is unsubstituted C5-C13 straight chain alkyl, Rb is unsubstituted C10 straight chain alkyl, unsubstituted C11 straight chain alkyl or unsubstituted C12 straight chain alkyl.

6. The compound of claim 5, wherein the compound of formula A is selected from a compound of formula II, the compound of formula II is selected from compound L0126, compound L0127 and compound L0128,

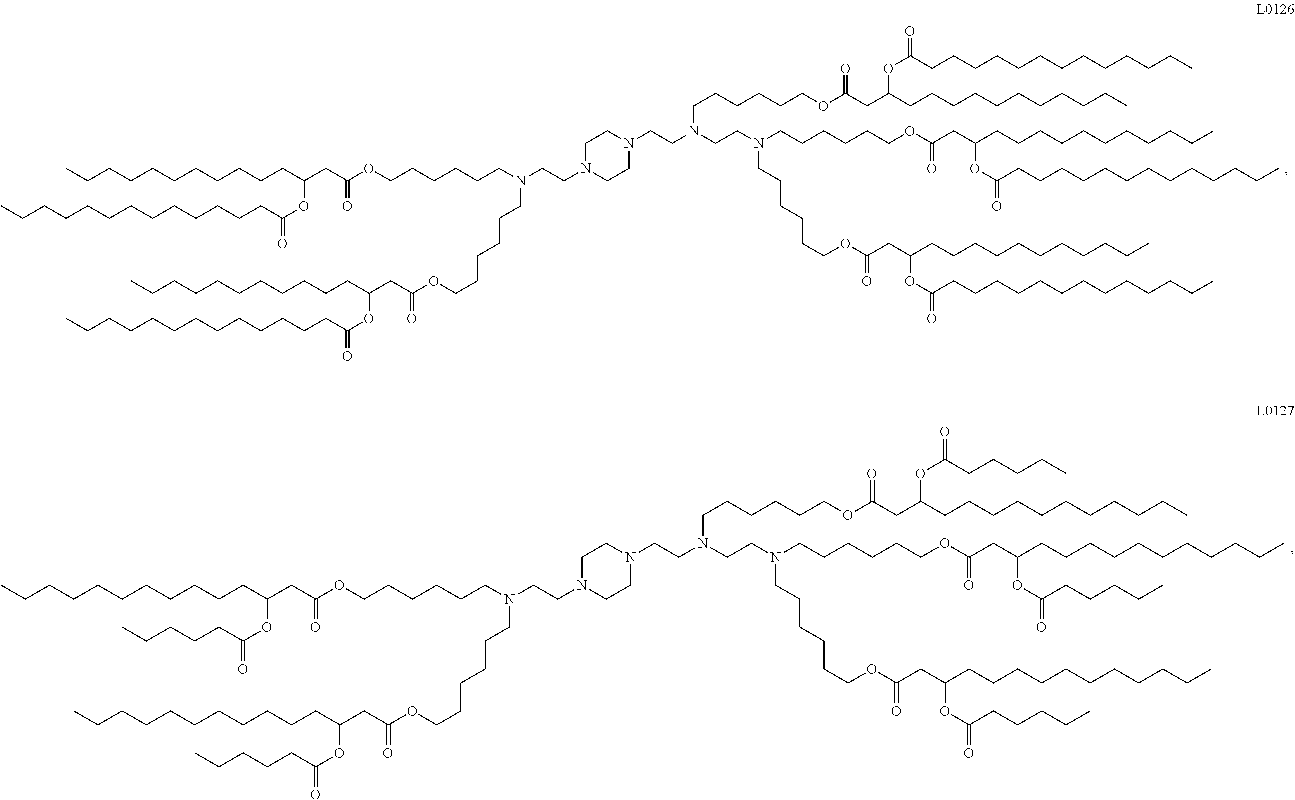

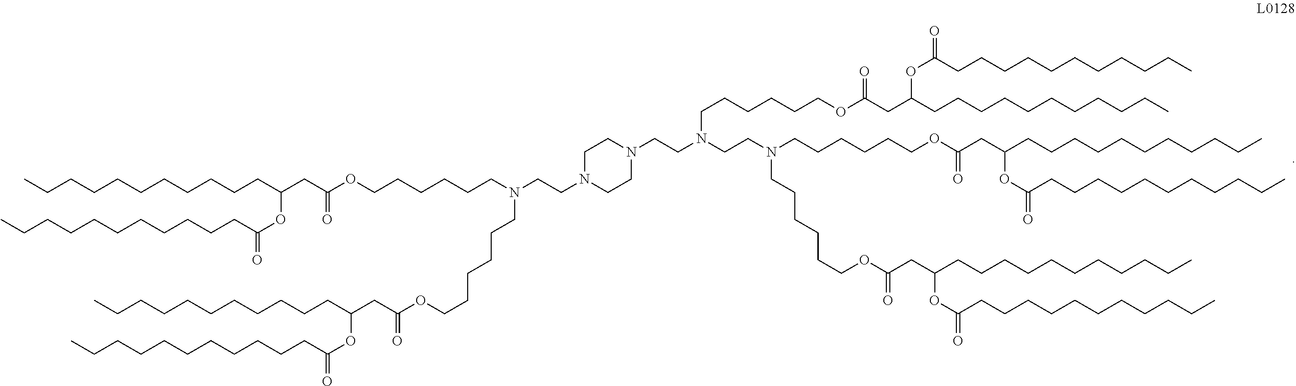 [SMILES: CCCCCCCCCCCC(=O)OC(CCCCCCCCCCC)CC(=O)OCCCCCCN(CCCCCCOC(=O)CC(CCCCCCCCCCC)OC(=O)CCCCCCCCCCC)CCN(CCCCCCOC(=O)CC(CCCCCCCCCCC)OC(=O)CCCCCCCCCCC)CCN1CCN(CCN(CCCCCCOC(=O)CC(CCCCCCCCCCC)OC(=O)CCCCCCCCCCC)CCCCCCOC(=O)CC(CCCCCCCCCCC)OC(=O)CCCCCCCCCCC)CC1]

7. The compound of claim 2, wherein the compound of formula A is selected from a compound of formula III, wherein a, b and c are the same and are an integer from 1 to 9; or
the compound of formula A is selected from a compound of formula III, wherein a, b and c are the same and are an integer from 1 to 5; or
the compound of formula A is selected from a compound of formula III, wherein a, b and c are the same and are an integer from 1 to 5; Ra is unsubstituted C5-C15 straight chain alkyl, Rb is unsubstituted C5-C15 straight chain alkyl; or
the compound of formula A is selected from a compound of formula III, wherein a, b and c are the same and are an integer from 3 to 5; Ra is unsubstituted C5-C11 straight chain alkyl, Rb is unsubstituted C8-C13 straight chain alkyl; or
the compound of formula A is selected from a compound of formula III, wherein a, b and c are the same and are an integer from 3 to 5; Ra is unsubstituted C5-C11 straight chain alkyl, Rb is unsubstituted C11 straight chain alkyl.

8. The compound of claim 7, wherein the compound of formula A is selected from a compound of formula III, the compound of formula III is selected from compound L0129 or compound L0130,

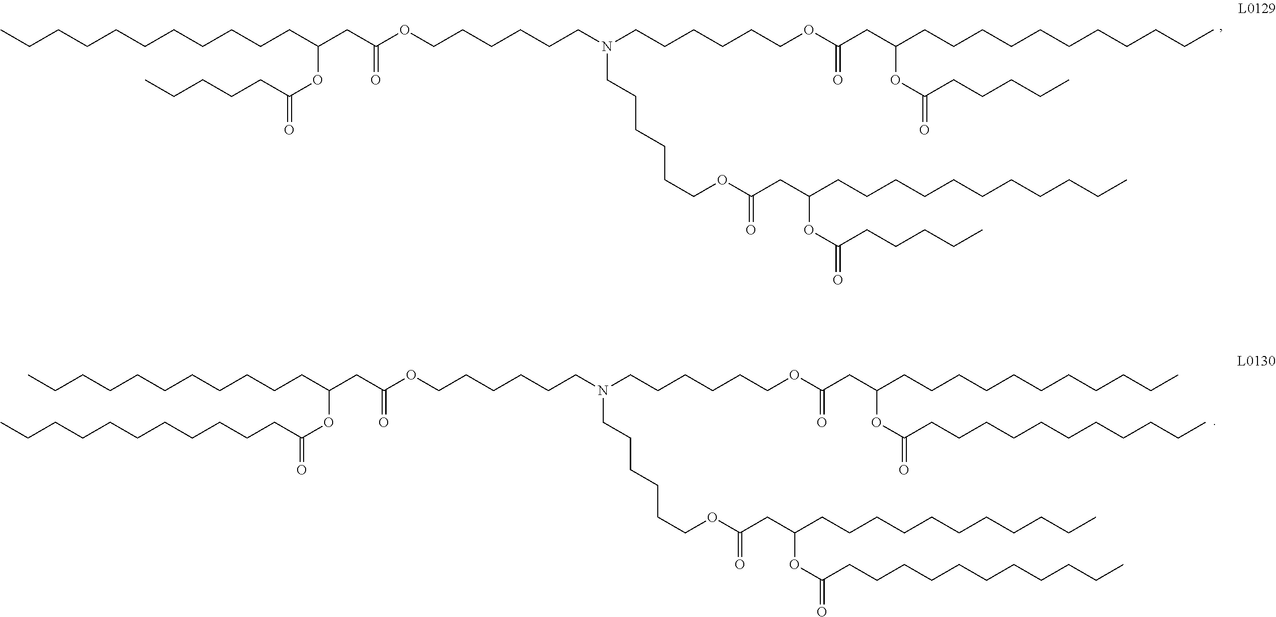

9. The compound of claim 2, wherein the compound of formula A is selected from a compound of formula IV, wherein a is an integer from 0 to 5, b is an integer from 3 to 10, c is an integer from 3 to 10, z is an integer from 5 to 10, Ra is unsubstituted C5-C15 straight chain alkyl, Rb is unsubstituted C5-C15 straight chain alkyl; or
the compound of formula A is selected from a compound of formula IV, wherein a is an integer from 0 to 3, b is an integer from 4 to 6, c is an integer from 4 to 6, z is an integer from 5 to 8, Ra is unsubstituted C5-C13 straight chain alkyl, Rb is unsubstituted C9-C13 straight chain alkyl; or
the compound of formula A is selected from a compound of formula IV, wherein a is 1, b is 5, c is 5, z is 6, Ra is unsubstituted C5-C13 straight chain alkyl, Rb is unsubstituted C10-C15 straight chain alkyl.

10. The compound of claim 2, wherein the compound of formula A is selected from a compound of IV, the compound of formula IV is selected from compound L0129 and compound L0131,

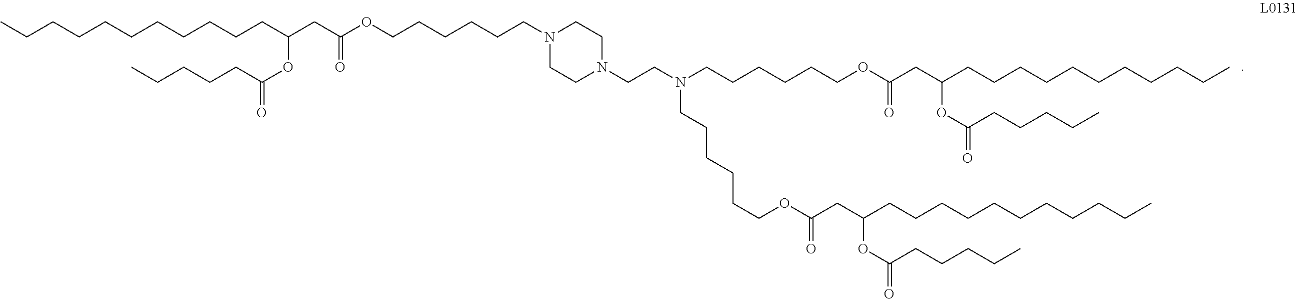 [SMILES: CCCCCCCCCCCC(CC(=O)OCCCCCCN(CCCCCCOC(=O)CC(CCCCCCCCCCC)OC(=O)CCCCC)CCN1CCN(CCCCCCOC(=O)CC(CCCCCCCCCCC)OC(=O)CCCCC)CC1)OC(=O)CCCCC]

11. A lipid compound nanoparticle comprising the following components: a compound of claim 1 and auxiliary materials;
optionally, the auxiliary material includes at least one of a PEG derivative, a lipid, a lipid-like substance, an alcohol, a saccharide and an inorganic salt.

12. The lipid compound nanoparticle of claim 11, wherein the PEG derivative includes at least one of PEG modified phosphatidylethanolamine, PEG modified phosphatidic acid, PEG modified ceramide, PEG modified dialkylamine, PEG modified diacylglycerol, PEG modified dialkyglycerol, PEG modified stearic acid and PEG modified phosphatidylserine; and/or
the PEG derivative includes at least one of 1,2-dimyristoyl-sn-glycero methoxypolyethylene glycol, 1,2-distearoyl-sn-glycero-3-phosphoethanolamine-N-(amino(polyethylene glycol)), dilauroyl phosphatidylethanolamine-polyethylene glycol, dimyristoyl phosphatidylethanolamine -polyethylene glycol, dipalmitoyl phosphatidylcholine polyethylene glycol, dipalmitoyl phosphatidylethanolamine-polyethylene glycol, PEG-distearoyl glycerol, PEG-dipalmitoyl, PEG-dioleoyl, PEG-distearoyl, PEG-diacylglycerol amide, PEG-dipalmitoyl phosphatidylethanolamine and PEG-1,2-dimyristol oxypropyl-3-amine; and/or
the PEG derivative includes at least one of PEG-c-DOMG, PEG-c-DMA, PEG-DMG, PEG-DSG, PEG-DAG, PEG-DLPE, PEG-DPPC, ALC-0159, PEG-Mal, DSPE-PEG-Mal, DMG-PEG2000, mPEG-DSPE, mPEG-STA, mPEG-PS, mPEG-DMPE and mPEG-DPPE; and/or
the lipid includes at least one of phospholipid and sterol; and/or
the phospholipid includes at least one of lecithin, 1,2-distearoyl-sn-glycero-3-phosphocholine, 1,2-dioleoyl-sn-glycero-3-phosphoethanolamine, 1,2-dioleoyl-sn-glycero-3-phosphocholine, 1,2-dimyristoyl-sn-glycero-3-phosphocholine, 1,2-dioleoyl-sn-glycero-3-phosphocholine, 1,2-dipalmitoyl-sn-glycero-3-phosphocholine, 1,2-diundecanoyl-sn-glycero-phosphocholine, 1-palmitoyl-2-oleoyl-sn-glycero-3-phosphocholine, Dilauroylphosphatidylcholine (DLPC), 1,2-Diheneicosanoyl-sn-glycero-3-phosphocholine (DUPC), 1-Palmitoyl-2-oleoyl-sn-glycero-3-phosphocholine (POPC), 1,2-Diisooctyl-OT-glycero-3-phosphocholine (18:0 Diether PC), 1-Oleoyl-2-cholesterylhemisuccinoyl-OT-glycero-S-phosphocholine (OChemSPC), 1,2-Dilinolenoyl-sn-glycero-3-phosphocholine, 1,2-Diarachidonoyl-sn-glycero-3-phosphocholine, 1,2-Didocosahexaenoyl-sn-glycero-3-phosphocholine, 1,2-Diphenylglyceryl-sn-glycero-3-phosphoethanolamine (ME16.0 PE), 1,2-Distearoyl-sn-glycero-3-phosphoethanolamine, 1,2-Dioctanoyl-sn-glycero-3-phosphoethanolamine, 1,2-Dilinoleoyl-sn-glycero-3-phosphoethanolamine, 1,2-Diarachidonoyl-sn-glycero-3-phosphoethanolamine, 1,2-Didocosahexaenoyl-sn-glycero-3-phosphoethanolamine, 1,2-Dioleoyl-sn-glycero-3-phospho-L -glycerol Sodium Salt (DOPG), Monopalmitoyl Phosphatidylcholine (MPPC), Myristoyl Stearoyl Phosphatidylcholine (MSPC), 1-palmitoyl-2-myristoyl-sn-glycero-3-phosphocholine (PMPC), 1-Palmitoyl-2-stearoyl-sn-glycero-3-phosphocholine (PSPC), 1-stearoyl-2-myristoyl-sn-glycero-3-phosphocholine (SMPC), 1-Stearoyl-2-palmitoyl-sn-glycero-3-phosphocholine (SPPC), 2-Oleoyl-1-stearoyl-sn-glycero-3-phosphocholine (SOPC), 1,2-Ditetradecyl-rac-glycero-3-phosphoethanolamine (DMPE), 1,2-Distearoyl-sn-glycero-3-phosphorylethanolamine (DSPE), and Sphingomyelin; and/or
the sterol includes at least one of cholesterol, lanosterol, 5 α-cholestan-3 β-alcohol, coprosterol, sitosterol, ergosterol, campesterol, stigmasterol, brassicasterol, tomatidine, ursolic acid and α-tocopherol.

13. The lipid compound nanoparticle of claim 11, wherein the lipid compound nanoparticle includes the compound of claim 1, a PEG derivative and a lipid; the lipid includes at least one of phospholipid and sterol; and/or
calculated on the total molar amount of each component of the lipid compound nanoparticle, the compound of claim 1 has an amount of 14.8 mol %-70.0 mol %; and/or
calculated on the total molar amount of each component of the lipid compound nanoparticle, the PEG derivative has an amount of 0.4 mol %-10 mol %; and/or
calculated on the total molar amount of each component of the lipid compound nanoparticle, the phospholipid has an amount of 5.0 mol %-50.0 mol %; and/or
calculated on the total molar amount of each component of the lipid compound nanoparticle, the sterol has an amount of 10.0 mol %-75.0 mol % or 15.0 mol %-75.0 mol%.

14. The lipid compound nanoparticle of claim 11, wherein a molar ratio of PEG derivative:phospholipid:sterol: the compound of claim 1 is (0.4-10.0):(5.0-50.0):(10.0-75.0):(14.8-70.0) or (0.4-10.0):(5.0-40.0):(15.0-75.0):(14.8-70.0); or
a molar ratio of PEG derivative:phospholipid:sterol: the compound of claim 1 is 2.50:16.00:16.50:65.00, 1.00:5.00:64.00:30.00, 0.40:8.00:56.60:35.00, 1.00:8.00:61.00:30.00, 1.20:12.00:38.30:48.50, 1.70:9.00:40.80:48.50, 2.50:16.00:21.50:60.00, 1.20:9.00:47.80:42.00, 1.00:16.00:34.50:48.50, 2.50:8.00:41.00:48.50, 1.50:8.00:25.50:65.00, 2.50:11.50:51.00:35.00, 1.50:8.00:30.50:60.00, 1.00:5.00:62.00:32.00, 0.60:5.00:54.40:40.00, 1.50:16.00:47.50:35.00, 1.63:12.99:45.38:40.00, 1.20:8.00:55.8:35.00, 1.30:8.00:55.70:35.00, 0.40:5.00:59.60:35.00, 1.00:20.00:37.00:42.00, 1.00:25.00:34.00:40.00, 3.20:12.00:29.80:55.00, 1.00:8.00:53.00:38.00, 2.50:9.50:33.00:55.00, 10.00:16.00:54.00:20.00, 3.00:30.00:42.00:25.00, 3.20:16.80:10.00:70.00, 3.00:17.00:25.00:55.00, 3.20:16.80:15.00:65.00, 4.20:11.00:70.00:14.80, 6.20:6.80:75.00:15.00, 1.50:11.50:38.50:48.50, 7.50:9.61:35.56:47.33, 3.00:9.50:32.50:55.00, 0.95:7.58:26.47:65.00, 1.40:11.15:38.95:48.50, 2.10:7.98:47.90:42.02, 3.00:6.00:43.00:48.00, 1.60:30.00:30.00:40.00, 1.60:35.00:35.00:30.00, 1.60:40.00:40.00:20.00, or 1.80:40.00:27.20:31.00; or
a molar ratio of PEG derivative:phospholipid:sterol: the compound of claim 1 is (0.4-1.5):(8.0-11.5):(38.5-56.6):(35.0-48.5); or
a molar ratio of PEG derivative:phospholipid:sterol: the compound of claim 1 is 0.40:8.00:56.60:35.00, 1.50:11.50:38.50:48.50 or 1.40:11.15:38.95:48.50.

15. A nucleic acid nanoparticle complex, comprising a nucleic acid and at least one lipid compound nanoparticle of claim 11.

16. The nucleic acid nanoparticle complex of claim 15, wherein a molar ratio of ionizable nitrogen atoms in the compound of claim 1 to phosphorus atoms of the nucleic acid in the nucleic acid nanoparticle complex is 5-50.

17. A pharmaceutical composition, comprising the lipid compound nanoparticle of claim 11 or the nucleic acid nanoparticle complex of claim 15, and a pharmaceutically acceptable adjuvant.

18. A method of delivering nucleic acids in vivo, comprising administering to a subject in need thereof the compound of claim 1, the lipid compound nanoparticle of claim 11, the nucleic acid nanoparticle complex of claim 15, or the pharmaceutical composition of claim 17.
